(19) United States
(12) Patent Application Publication
(10) Pub. No.: US 2007/0204352 A1
(43) Pub. Date: Aug. 30, 2007

(54) Regulators of protein misfolding and aggregation and methods of using the same

(76) Inventors: Guy A. Caldwell, Northport, AL (US); Kim A. Caldwell, Northport, AL (US)

Correspondence Address: KING & SPALDING LLP, 1180 PEACHTREE STREET, ATLANTA, GA, 30309-3521, US

(21) Appl. No.: 11/364,612

(22) Filed: Feb. 27, 2006

Related U.S. Application Data
(60) Provisional application No. 60/656,334, filed on Feb. 25, 2005.
(60) Provisional application No. 60/681,890, filed on May 17, 2005.
(60) Provisional application No. 60/738,761, filed on Nov. 21, 2005.
(60) Provisional application No. 60/749,910, filed on Dec. 12, 2005.

(51) Int. Cl. A01K 67/027; G01N 33/567

(52) U.S. Cl. 800/12; 435/7.2

(57) ABSTRACT

Polynucleotide molecules and the proteins encoded by the molecules, diagnostic and treatment methods for neurological disorders characterized by protein aggregation are provided. Genes are described herein that affect the misfolding of, and subsequent aggregation of, aggregation-prone proteins such as alpha-synuclein and have implications for the diagnosis and treatment of neurological diseases related to protein aggregation such as Parkinson's disease. Knockdown of expression of the genes described herein using RNAi results in alpha-synuclein protein aggregation in a C. elegans model of protein aggregation. Dopaminergic neuroprotection after exposure to the neurotoxin 6-OHDA or overexpression of alpha-synuclein may also be provided by overexpression of proteins. Knowledge of genes relating to protein misfolding and aggregation provides powerful means to develop diagnostic screening methods, mutation analysis and drug design information for the development of novel therapeutic and neuroprotective compounds to treat neurodegenerative diseases such as Parkinson's disease.

### CROSS REFERENCES TO RELATED APPLICATIONS

The present application claims the benefit of U.S. Provisional Patent Applications 60/656,334 filed Feb. 25, 2005, 60/738,761 filed Nov. 21, 2005, and 60/749,910 filed Dec. 12, 2005 which are incorporated by reference herein.

### FIELD OF THE INVENTION

This invention relates to polynucleotide molecules encoding neuroprotective proteins that regulate protein aggregation and methods of using the same. More specifically this invention relates to methods of using polynucleotide molecules and neuroprotective proteins encoded by them to prevent protein misfolding and dopaminergic neurodegeneration.

### BACKGROUND OF THE INVENTION

Neuronal malfunction and damage may be caused by toxic, aggregation-prone proteins and a number of neurological disorders are characterized by such conditions. These include disorders such as amyotrophic lateral sclerosis, Alzheimer's disease, Parkinson's disease, prion disease, polyglutamine expansion diseases, spincocerebellar ataxia, spinal & bulbar muscular atrophy, spongiform encephalopathy, tauopathy, Huntington's disease, or dystonia. Proteins and the genes encoding them have been identified that code for toxic, aggregation-prone proteins which cause these disorders. Normal metabolic enzymes recycle proteins creating a perpetual cycle of synthesis and degradation. Mutations in these genes result in abnormal accumulation and degradation of misfolded proteins. These misfolded proteins are known to result in neuronal inclusions and plaques which may be indicative of neuronal damage. Therefore, the understanding of the cellular mechanisms and the identification of the molecular tools required for the reduction, inhibition, and amelioration of such misfolded proteins is critical. Furthermore, an understanding of the effects of protein misfolding and aggregation on neuronal survival will allow the development of rational, effective treatment for these disorders.

Parkinson's Disease is a neurological disorder characterized by limb tremors, slow or no movement, stiff limbs, shuffling walk, and a stooped posture. Other symptoms may include depression, personality changes, dementia, sleep disturbances, speech impairments, or sexual difficulties. These conditions progressively become more severe. The symptoms are a result of neuronal degeneration in the basal ganglia specifically in the substantia nigra with secondary degeneration in the raphe nuclei and the locus ceruleus. This neuronal degeneration is commonly associated with the misfolding and subsequent aggregation of the protein alpha-synuclein. Neuronal degeneration in the substantia nigra leads to a reduction of the neurotransmitter, dopamine, resulting in deficits in neurotransmission causing severe impairment of motor skills.

Mutant forms of alpha-synuclein are thought to increase the propensity for misfolding and induce other proteins to incorporate into the aggregates as well. Deficits in protein degrading enzymes may also contribute to protein accumulation, aggregation and alter cellular homeostasis. These aggregates are known as Lewy bodies and are comprised primarily of alpha-synuclein. They constitute the pathological hallmark of Parkinson's disease, Dementia with Lewy bodies and other neurodegenerative diseases. Lewy bodies are similar to the beta-amyloid plaques found in Alzheimer's patients. In fact, alpha-synuclein is also the largest component of these Alzheimer-related plaques. The presence of Lewy bodies in Parkinson's brains is coincident with loss of dopaminergic neurons and subsequent loss of motor control. The presence of alpha-synuclein in neurofibrillary tangles has also been implicated in Alzheimer's disease, Pick's disease, Progressive Supranuclear Palsy, and Corticobasal Degeneration.

A major obstacle surrounding neurodegenerative disorders is that patients are unaware that a neuronal environment that contributes to neuronal degeneration is developing until the point where clinical symptoms manifest. By the time clinical symptoms manifest there is already tremendous neuronal loss and the neuronal environment is significantly hostile to the survival of neurons. The lack of reliable early detection methods for protein aggregation or neuronal loss allows these degenerative diseases to develop unmonitored until a point where treatment may be ineffective or unnecessary as neuronal loss has already occurred. Furthermore, even if reliable early detection methods were available, current therapies are ineffective for long-term treatment of these neurodegenerative diseases and novel drugs and treatment methods are necessary.

An understanding of the molecular mechanisms and protein regulators for aberrant protein aggregation is required to develop improved methods to diagnose these disorders at early stages prior to significant neuronal destruction and to provide model systems for drug design and development. Compounds that target specific genes and gene products related to protein aggregation may be screened for and developed using model systems. It is also necessary to understand the mechanisms of neurodegeneration and develop neuroprotective compounds that may prevent or attenuate neuronal loss until more effective treatments of the root cause of aberrant protein misfolding and aggregation may be developed.

### SUMMARY OF THE INVENTION

The present invention is directed to novel methods of using polynucleotide molecules and the proteins encoded by the molecules for use in diagnostic and treatment methods for neurological disorders characterized by neuron malfunction, neurodegeneration or protein misfolding and subsequent aggregation. Specifically, a number of genes are described herein that affect the misfolding of, and subsequent aggregation of aggregation-prone proteins and have implications for the diagnosis and treatment of neurological diseases related to protein aggregation. The genes described herein result in an increase in protein misfolding and aggregation, specifically of alpha-synuclein when knocked down in an RNAi screen. Knowledge of genes relating to this process provides powerful means to develop diagnostic screening methods, mutation analysis and drug design information for the development of novel therapeutic and neuroprotective compounds. These methods include modulating the activity of a number of proteins to reduce or prevent protein misfolding or provide neuroprotection. These include ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins.

Accordingly, an object of the present invention is to provide methods and compositions for detecting and treating neurological disorders related to protein misfolding and aggregation.

It is another object of the present invention to provide methods and compositions for detecting and treating specifically Parkinson's disease or disorders due to alpha-synuclein misfolding and aggregation.

It is another object of the invention to provide methods of detecting the presence or absence of a neurodegenerative disorder in a human wherein the disorder is characterized by changes in expression levels or one or more mutations in a gene related to protein misfolding and aggregation.

It is another object of the invention to provide methods to detect mutations or polymorphisms in other neuronal genes implicated in conferring a particular phenotype which gives rise to overt clinical symptoms in a mammal that are consistent with the neuroanatomical expression of the gene.

It is another object of the invention to provide diagnostic methods for neurological disorders related to protein misfolding and aggregation in humans. Preferably, a method of diagnosing the presence or absence of the disorder; predicting the likelihood of developing or a predisposition to develop the disorder in a human is provided herein.

It is another object of the invention to provide a method of identifying a mutation or polymorphism in a neuronal gene relating to protein aggregation that confers increased susceptibility to a neuronal disease.

It is another object of the invention to provide a method of screening for a compound that reduces, inhibits, ameliorates, or prevents protein misfolding and aggregation by comparing the amount of protein misfolding and aggregation in the presence of the compound to the amount of protein misfolding and aggregation in the absence of the compound.

It is another object of the invention to provide a method of screening for a compound that reduces, inhibits, ameliorates, or prevents neurodegeneration by comparing the amount of neurodegeneration in the presence of the compound to the amount of neurodegeneration in the absence of the compound.

It is another object of the invention to provide methods of designing and developing therapeutic compounds to provide neuroprotection to neurons susceptible to conditions promoting protein aggregation or compounds to prevent or attenuate protein misfolding and aggregation or compounds to solubilize protein aggregates.

It is another object of the invention to provide a method of reducing, arresting, alleviating, ameliorating, or preventing cellular dysfunction as a result of protein aggregation.

It is another object of the invention to provide pharmaceutical formulations in an effective amount of a composition to reduce protein misfolding and aggregation in an animal in need of treatment or to provide neuroprotection.

The present invention is also directed to methods of using polynucleotide molecules and polypeptides encoded by them to provide neuroprotection to neurons susceptible to conditions promoting protein misfolding and aggregation.

It is another object of the invention to provide methods for making a medicament for treating neurological diseases associated with protein misfolding and aggregation.

It is another object of the invention to provide transgenic animals for use in screening novel therapies to treat neurological disorders.

It is another object of the invention to provide a kit for diagnosing the presence or absence of a neurodegenerative disorder in a human comprising one or more reagents for detecting a mutation in a gene in a sample obtained from the human.

These and other objects, features and advantages of the present invention will become apparent after a review of the following detailed description of the disclosed embodiment and the appended claims.

### BRIEF DESCRIPTION OF DRAWINGS

FIG. 1a provides a diagram showing the results of screening alpha-synuclein::GFP+TOR-2 transgenic nematodes for positive candidates.

FIG. 1b provides a diagram showing the distribution of positive candidates from the primary screen of alpha-synuclein::GFP+TOR-2 transgenic nematodes.

FIG. 2a provides a diagram showing the overlap of candidates identified from microarray experiments of the co-expression of DJ-1 and PINK1.

FIG. 2b provides a diagram showing the 17 candidates listed in Table I Functional distribution of these genes from screening demonstrates a significant overlap exists in their classification.

FIG. 3a provides a graph showing the effects of C. elegans M7.5 protein expression on neuroprotection in dopamine neurons after 6-OHDA exposure over time as animals age.

FIG. 3b provides a graph showing the comparison of neuroprotective qualities between TOR-2 and the autophagy protein M7.5 on 6-OHDA induced neurodegeneration of dopamine neurons.

FIG. 4 provides a graph showing the neuroprotective capacity of TOR-2 and the autophagy protein M7.5 against the degeneration of dopamine neurons induced by overexpression of human alpha-synuclein.

### DETAILED DESCRIPTION OF THE INVENTION

The present invention may be understood more readily by reference to the following detailed description of specific embodiments included herein. Although the present invention has been described with reference to specific details of certain embodiments, thereof, it is not intended that such details should be regarded as limitations upon the scope of the invention. The text of the references mentioned herein are hereby incorporated by reference in their entirety.

Neurons are particularly vulnerable to the toxic effects of mutant or misfolded proteins. Based on an understanding of the normal cellular mechanisms for disposing of unwanted and potentially noxious proteins, the present invention provides unique methods and compositions for negating the effects on neurons of misfolded or aggregated proteins. Mutant or misfolded proteins may result in the damage, degeneration or death of neurons but may also cause neuron malfunction where the neuron survives but cellular processes are impaired that result in the onset of clinical symptoms of neurological disease.

In the present specification and the appended claims, the singular forms “a”, “an”, and “the” include plural references unless the context clearly dictates otherwise.

It is to be understood that although the following discussion is specifically directed to human patients, the teachings are also applicable to any animal that expresses a protein from Table I. The term “mammalian,” as defined herein, refers to any vertebrate animal, including monotremes, and marsupials. Examples of mammalian species include primates (e.g., humans, monkeys, chimpanzees, baboons), rodents (e.g., rats, mice, guinea pigs, hamsters) and ruminants (e.g., cows, horses).

“Treating” within the scope of the present invention comprises reducing, inhibiting, ameliorating, or preventing symptoms or molecular events associated with an abnormality such as neurodegenerative disease, including but not limited to, Parkinson's disease. Preferably, protein aggregation, cellular dysfunction as a result of protein misfolding and aggregation and protein-aggregation-associated diseases may be treated.

“Neurological disorders” comprise clinical conditions characterized by the degeneration and/or loss of neurons. Included in these disorders are amyotrophic lateral sclerosis, Alzheimer's disease, Parkinson's disease, prion disease, frontotemporal dementia, polyglutamine expansion diseases, spincocerebellar ataxia, spinal & bulbar muscular atrophy, spongiform encephalopathy, tauopathy, Huntington's disease, dystonia and the like.

As used herein, the term “worm” refers to a model system used to study protein aggregation of the present invention where the model organism is from the phylum nematoda. Included within this meaning is the specific nematode Caenorhabditis elegans or C. elegans.

Correct folding requires proteins to assume one particular structure from a constellation of possible but incorrect conformations. The failure of polypeptides to adopt their proper structure is a major threat to cell function and viability. Misfolded proteins may be toxic in and of themselves and form aggregates that may have very serious or even lethal consequences. Consequently, elaborate systems have evolved to protect cells from the deleterious effects of misfolded proteins.

“Protein” within the scope of the present invention includes full-length proteins, homologues, proteins with altered glycosylation, protein fragments, splice variants, functionally equivalent variants, mutants and conservative substitutions thereof that retain substantially the same function as the wild type protein.

“Protein aggregation” within the scope of the present invention includes the phenomenon of at least two polypeptides contacting each other in a manner that causes either one of the polypeptides to be in a state of desolvation. This may also include a loss of the polypeptide's native function or activity.

“Protein-aggregation-associated disease” within the scope of the present invention includes any disease, disorder, and/or affliction, protein-aggregation-associated disease including neurodegenerative disorders.

Reference is made to standard textbooks of molecular biology that contain definitions and methods and means for carrying out basic techniques, encompassed by the present invention. See, for example, Sambrook et al., Molecular Cloning: A Laboratory Manual, Third Edition, Cold Spring Harbor Laboratory Press, New York (2001), Current Protocols in Molecular Biology, Ausebel et al (eds.), John Wiley & Sons, New York (2001) and the various references cited therein.

The present invention provides a number of polynucleotides that encode proteins relating to protein misfolding/aggregation and neuroprotection. Some candidate genes encode hypothetical proteins with a function or activity that until now was unknown. However, the present invention establishes that at least one common function or activity of these proteins is the prevention of protein misfolding and aggregation. A reduction in the activity of these proteins using RNAi results in protein misfolding and alpha-synuclein aggregates in a C. elegans model. Alternations in these proteins and the polynucleotides encoding them that reduce expression and or activity should also result in protein misfolding and aggregation.

Some of these proteins also provide neuroprotection to neurons such as dopamine-containing neurons. Accordingly, the present invention provides a novel approach for therapeutic intervention in neurodegenerative disease comprising the use of polynucleotides described herein for neuroprotection of dopamine containing neurons; as such the present invention provide another avenue for developing treatments for Parkinson's disease. Genes encoding proteins that impart neuroprotective qualities in dopaminergic neurons can be used to develop gene and protein therapies, antibody therapies, and in the design and screening for new drugs to provide neuroprotection of dopamine neurons. Similarly, alterations in these molecules may predispose neurons to damage and death under adverse conditions. The proteins encoded by these genes include UPS components, components of the autophagy machinery, molecular chaperones, transcription factors, vesicular trafficking proteins, Mn²⁺/Fe²⁺ transporters, HSPC117 proteins, acetylcholine receptor subunits, DJ-1 proteins and PINK-1 proteins. A list of these proteins is provided in Table I.

TABLE I

| C. elegans | | | |
|---|---|---|---|
| ORF- | | | |
| identifier | Predicted function | Human homolog | E-value |
| Y37A1B.13 | ATPase of the AAA+ | Torsin A, responsible for | 2.4e−63 |
| (tor-2) | superfamily, component of | early-onset dystonia | |
| | Lewy bodies (chaperone) | | |
| F57B10.5 | Emp24/gp25L/p24 family of | CGI-109 protein | 2.7e−58 |
| | membrane trafficking proteins | | |
| | (vesicular trafficking) | | |
| R05D11.6 | Transcription factor | Hypothetical protein MGC | 9.1e−05 |
| | | 13017 | |
| F16A11.2 | Uncharacterized conserved | Hypothetical protein | 2e−207 |
| | protein (HSPC 117 protein) | HSPC117 | |
| F26E4.11 | E3 ubiquitin ligase | Autocrine motility factor | 1.9e−39 |
| | (UPS protein) | receptor, isoform 2 | |
| B0432.2 | Unknown Function (DJ-1 protein) | PD-related protein, DJ-1 | 1.3e−45 |
| (DJ-1) | | | |
| EEED8.9 | BRPK/PTEN-induced protein | Splice isoform 1; | 2.1e−53 |
| (PINK-1) | kinase, PD-related protein | Serine/threonine kinase | |
| | (PINK-1 protein) | PINK1, mitochondrial | |
| | | precursor | |
| C35D10.2 | RGS-GAIP interacting protein | RGS19-interacting protein 1 | 1.1e−49 |
| | GIPC (autophagy protein) | | |
| F11H8.1 | NEDD8-activating complex, | Ubiquitin-activating | 5.3e−117 |
| (rfl-1) | catalytic component UBA3 | enzyme E1C | |
| | (UPS protein) | | |
| T13A10.2 | Predicted E3 ubiquitin ligase | Tripartite motif protein 2 | 0.00012 |
| | (UPS protein) | | |
| M7.5 | Ubiquitin activating E1 | E1-like protein | 7.4e−87 |
| | enzyme-like protein, | | |
| | (autophagy protein) | | |
| T08D2.4 | Hypothetical protein with | Tripartite motif protein 32 | 2.1e−06 |
| | RING finger motif (E3 ligase) | | |
| | (UPS protein) | | |
| C24G6.5 | Molecular chaperone (DnaJ | DnaJ homolog subfamily A | 1.9e−77 |
| (dnj-6) | superfamily) (chaperone) | member 2 | |
| T07F12.4 | Serine/threonine-protein | ULK2 protein | 4.3e−29 |
| | kinase (autophagy protein) | | |
| F32A6.3 | Vacuolar assembly/sorting | Splice isoform 1 of | 73-82 |
| | protein VPS41 (autophagy protein) | vacuolar assembly protein | |
| | | VPS41 homolog | |
| K11G12.4 | Mn²⁺ and Fe²⁺ transporters of | Divalent metal transporter | 3e−148 |
| (smf-1) | the NRAMP family | | |
| | (Mn/Fe transporter) | | |
| F48E3.7 | Acetylcholine receptor subunit | Neuronal acetylcholine | 8e−50 |
| (acr-22) | | receptor protein, alpha 9 | |
| | | chain precursor | |

Within the context of the present invention “isolated” or “purified” means separated out of its natural environment, which is also substantially free of other contaminating proteins, polynucleotides, and/or other biological materials often found in cell extracts.

Within the context of the present invention “polynucleotide” in general relates to polyribonucleotides and polydeoxyribonucleotides, it being possible for these to be non-modified RNA or DNA or modified RNA or DNA. Polynucleotide molecules may include genes and RNA that encode proteins or non-coding RNA or DNA.

The molecules shown in Table I are listed by the name of the C. elegans open reading frame (ORF) identifier but the present invention should not be limited to only C. elegans sequences. Other species homologs of the molecules listed in Table I are contemplated for use in the present invention, specifically human homologs. The sequences of C. elegans and corresponding human genes and proteins are provided herein. Corresponding C. elegans nucleotide and protein sequences as well as human nucleotide and protein sequences are provided in Table II.

TABLE II

| C. elegans | | | |
|---|---|---|---|
| ORF | | SEQ ID | |
| Identifier | Name | # | Source & Type of Sequence |
| Y37A1B.13 | tor-2 | 1 | C. elegans nucleotide |
| | tor-2 | 2 | C. elegans protein |
| | torsinA | 3 | Human nucleotide |
| | torsinA | 4 | Human protein |
| F57B10.5 | | 5 | C. elegans nucleotide |
| | | 6 | C. elegans protein |
| | CGI-109 | 7 | Human nucleotide |
| | CGI-109 | 8 | Human protein |
| R05D11.6 | | 9 | C. elegans nucleotide |
| | | 10 | C. elegans protein |
| | MCG13017 | 11 | Human nucleotide |
| | MCG13017 | 12 | Human protein |
| F16A11.2 | | 13 | C. elegans nucleotide |
| | | 14 | C. elegans protein |
| | HSPC117 | 15 | Human nucleotide |
| | HSPC117 | 16 | Human protein |
| F26E4.11 | | 17 | C. elegans nucleotide |
| | | 18 | C. elegans protein |
| | | 19 | Human nucleotide |
| | | 20 | Human protein |
| B0432.2 | | 21 | C. elegans nucleotide |
| | | 22 | C. elegans protein |
| | DJ-1 | 23 | Human nucleotide |
| | DJ-1 | 24 | Human protein |
| EEED8.9 | PINK-1 | 25 | C. elegans nucleotide |
| | PINK-1 | 26 | C. elegans protein |
| | PINK-1 | 27 | Human nucleotide |
| | PINK-1 | 28 | Human protein |
| C35D10.2 | | 29 | C. elegans nucleotide |
| | | 30 | C. elegans protein |
| | RGS19 | 31 | Human nucleotide |
| | RGS19 | 32 | Human protein |
| F11H8.1 | rfl-1 | 33 | C. elegans nucleotide |
| | rfl-1 | 34 | C. elegans protein |
| | E1C | 35 | Human nucleotide |
| | E1C | 36 | Human protein |
| T13A10.2 | | 37 | C. elegans nucleotide |
| | | 38 | C. elegans protein |
| | | 39 | Human nucleotide |
| | | 40 | Human protein |
| M7.5 | | 41 | C. elegans nucleotide |
| | | 42 | C. elegans protein |
| | E1-like | 43 | Human nucleotide |
| | E1-like | 44 | Human protein |
| T08D2.4 | | 45 | C. elegans nucleotide |
| | | 46 | C. elegans protein |
| | | 47 | Human nucleotide |
| | | 48 | Human protein |
| C24G6.5 | dnj-6 | 49 | C. elegans nucleotide |
| | dnj-6 | 50 | C. elegans protein |
| | DnaJ | 51 | Human nucleotide |
| | DnaJ | 52 | Human protein |
| T07F12.4 | | 53 | C. elegans nucleotide |
| | | 54 | C. elegans protein |
| | ULK2 | 55 | Human nucleotide |
| | ULK2 | 56 | Human protein |
| F32A6.3 | | 57 | C. elegans nucleotide |
| | | 58 | C. elegans protein |
| | VPS41 | 59 | Human nucleotide |
| | VPS41 | 60 | Human protein |
| K11G12.4 | smf-1 | 61 | C. elegans nucleotide |
| | smf-1 | 62 | C. elegans protein |
| | | 63 | Human nucleotide |
| | | 64 | Human protein |
| F48E3.7 | acr-22 | 65 | C. elegans nucleotide |
| | acr-22 | 66 | C. elegans protein |
| | | 67 | Human nucleotide |
| | | 68 | Human protein |

One skilled in the art will realize that organisms other than humans will also contain such genes (for example, eukaryotes; more specifically, mammals (preferably, gorillas, rhesus monkeys, and chimpanzees), rodents, worms (preferably. C. elegans), insects (preferably, D. melanogaster), birds, fish, yeast, and plants). The invention is intended to include, but is not limited to, nucleic acid molecules isolated from the above-described organisms that encode the proteins listed in Table I.

There is a remarkable degree of evolutionary conservation for many of these genes demonstrating a high homology for a protein between species. For example, human HSPC117 is homologous to C. elegans F16A11.2 and also Drosophila melanogaster (SEQ ID NO: 69 and 70), Danio rerio (SEQ ID NO: 71 and 72), bovine (SEQ ID NO: 73 and 74), mouse (SEQ ID NO: 75 and 76), and rat (SEQ ID NO: 77 and 78) genes/proteins. All of these sequences have e-value of essentially zero, demonstrating that this gene is highly conserved throughout evolution. In view of the high degree of homology in structure, these sequences should have the same function for reducing neurodegeneration, protein misfolding and aggregation when expressed at appropriate levels.

Isolated nucleic acid molecules of the present invention also include chemically synthesized nucleic acid molecules. For example, a nucleic acid molecule with the nucleotide sequence which codes for the expression product of a gene can be designed and, if necessary, divided into appropriate smaller fragments. Then an oligomer which corresponds to the nucleic acid molecule, or to each of the divided fragments, can be synthesized. Such synthetic oligonucleotides can be prepared synthetically (Matteucci et al., 1981, J Am. Chem. Soc. 103:3185-3191) or by using an automated DNA synthesizer. An oligonucleotide can be derived synthetically or by cloning. If necessary, the 5′ ends of the oligonucleotides can be phosphorylated using T4 polynucleotide kinase. Kinasing the 5′ end of an oligonucleotide provides a way to label a particular oligonucleotide by, for example, attaching a radioisotope (usually ³²P) to the 5′ end. Subsequently, the oligonucleotide can be subjected to annealing and ligation with T4 ligase or the like.

Furthermore, DNA sequences, which are prepared by the polymerase chain reaction (PCR) using primers, which result from the sequences of Table II are useful in the present invention. Such oligonucleotides typically have a length of at least 15 nucleotides.

Amino acid sequences and use thereof, which result in a corresponding manner from the proteins listed in Table I are contemplated in the present invention.

“Consisting essentially of”, in relation to a nucleic acid sequence, is a term used hereinafter for the purposes of the specification and claims to refer to substitution of nucleotides as related to third base degeneracy. As appreciated by those skilled in the art, because of third base degeneracy, almost every amino acid can be represented by more than one triplet codon in a coding nucleotide sequence. Further, minor base pair changes may result in variation (conservative substitution) in the amino acid sequence encoded, are not expected to substantially alter the biological activity of the gene product. Thus, a nucleic acid sequencing encoding a protein or peptide as disclosed herein, may be modified slightly in sequence (e.g., substitution of a nucleotide in a triplet codon), and yet still encode its respective gene product of the same amino acid sequence.

“Alterations” in the sequence of a polynucleotide as used herein refers to differences in expression levels of a sequence such as increases or decreases caused by knockout or knockdown of a gene. Also included are differences in the sequence itself that have an effect on the proper protein folding and neuroprotection afforded by the wild type protein. Such alterations include increases or decreases in expression, mutations, truncations and deletions of the polynucleotide molecule or protein. Consequently, DNA sequences which hybridize with the polynucleotide molecules that encode ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins or fragments thereof are a constituent of the invention.

The skilled artisan will find instructions for identifying DNA sequences by means of hybridization can be found by the expert, inter alia, in the handbook “The DIG System User Guide for Filter Hybridization” from Boehringer Mannheim GmbH (Mannheim, Germany, 1993) and in Liebl et al. (International Journal of Systematic Bacteriology 41: 255-260 (1991)). The hybridization takes place under stringent conditions, that is to say only hybrids in which the probe and target sequence, i.e. the polynucleotides treated with the probe, are at least 70% identical are formed. It is known that the stringency of the hybridization, including the washing steps, is influenced or determined by varying the buffer composition, the temperature and the salt concentration. The hybridization reaction is preferably carried out under a relatively low stringency compared with the washing steps (Hybaid Hybridisation Guide, Hybaid Limited, Teddington, UK, 1996).

A 5×SSC buffer at a temperature of approx. 50° C.-68° C., for example, can be employed for the hybridization reaction. Probes can also hybridize here with polynucleotides, which are less than 70% identical to the sequence of the probe. Such hybrids are less stable and are removed by washing under stringent conditions. This can be achieved, for example, by lowering the salt concentration to 2×SSC and optionally subsequently 0.5×SSC (The DIG System User's Guide for Filter Hybridisation, Boehringer Mannheim, Mannheim, Germany, 1995) with a temperature of approx. 50° C.-68° C. being established. It is optionally possible to lower the salt concentration to 0.1×SSC. Polynucleotide fragments which are, for example, at least 70% or at least 80% or at least 90% to 95% identical to the sequence of the probe employed can be isolated by increasing the hybridization temperature stepwise from 50° C. to 68° C. in steps of approx. 1-2° C. Further instructions on hybridization are obtainable on the market in the form of so-called kits (e.g. DIG Easy Hyb from Roche Diagnostics GmbH, Mannheim, Germany, Catalogue No. 1603558).

A “mutation” is any detectable change in the genetic material which can be transmitted to daughter cells and possibly even to succeeding generations giving rise to mutant cells or mutant individuals. A mutation can be any (or a combination of) detectable, unnatural change affecting the chemical or physical constitution, mutability, replication, phenotypic function, or recombination of one or more deoxyribonucleotides; nucleotides can be added, deleted, substituted for, inverted, or transposed to new positions with and without inversion. The term “mutation”, as used herein, can also refer to any modification in a nucleic acid sequence encoding one of the proteins described herein. For example, the mutation can be a point mutation or the addition, deletion, insertion and/or substitution of one or more nucleotides or any combination thereof. The mutation can be a missense or frameshift mutation. Modifications can be, for example, conserved or non-conserved, natural or unnatural. It is furthermore known that changes on the N and/or C terminus of a protein cannot substantially impair or can even stabilize the function thereof. Information in this context can be found by the expert, inter alia, in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (BioTechnology 6:1321-1325 (1988)) and in known textbooks of genetics and molecular biology. Mutations can be isolated by hybridization with polynucleotide molecules corresponding to the polynucleotide molecules listed in Table II or fragments thereof.

The present invention also contemplates methods of using a number of polypeptide molecules such as proteins that are directed to the prevention of protein misfolding and methods of their use. The proteins are described in Table I and the amino acid sequences are listed in Table II. These proteins are preferably purified or isolated to a substantially pure state free of contaminating proteins, polynucleotides or other contaminating compounds.

As used herein, “alterations” in a protein refers to the changes in the ability of a protein to assist in proper protein folding and provide neuroprotection as afforded by a wild type protein. Such alterations may include for example changes in protein expression, mutations in the protein sequence and alternatively spliced forms although other alterations that change the activity of the protein are contemplated.

In another embodiment, the polypeptide has the amino acid sequence set forth in Table II or a mutant or species variation thereof; or at least 70% identity, further at least 80% identity or and even further at least 90% identity thereof (preferably, at least 90%, 95%, 96%, 97%, 98%, or 99% identity or at least 95%, 96%, 97%, 98%, or 99% similarity thereof), or at least 6 contiguous amino acids thereof (preferably, at least 10, 15, 20, 25, or 50 contiguous amino acids thereof).

The proteins of the present invention may be provided in a glycosylated as well as an unglycosylated form. Preparation of glycosylated proteins or fragments thereof is known in the art and typically involves expression of the recombinant DNA encoding the peptide in a eukaryotic cell. Likewise, it is generally known in the art to express the recombinant DNA encoding the peptide in a prokaryotic (e.g., bacterial) cell to obtain a peptide, which is not glycosylated. These and other methods of altering carbohydrate moieties on glycoproteins are found in Essentials of Glycobiology (1999), Edited By Ajit Varki, Cold Spring Harbor Laboratory Press, Cold Spring Harbor, N.Y., the contents of which are incorporated herein by reference.

Polypeptide molecules are also contemplated that consist essentially of the polypeptide sequences for the proteins listed in Table I.

The proteins of the present invention may contain one or more protected amino acid residues. The protected amino acid is an amino acid whose functional group or groups is/are protected with a protecting group or groups by a known method and various protected amino acids are commercially available. The proteins or fragments thereof may also contain one or more modified amino acids. A list of such amino acids can be found in U.S. Patent Publication 2003/0235823 which is incorporated herein by reference in its entirety.

While the site for introducing an amino acid sequence variation is predetermined, the mutation itself need not be predetermined. For example, to optimize the performance of a particular polypeptide with respect to a desired activity, random mutagenesis can be conducted at a target codon or region of the polypeptide, and the expressed variants can be screened for the optimal desired activity. Techniques for making substitution mutations at predetermined sites in DNA having a known sequence are well known, e.g., site-specific mutagenesis.

Amino acid sequence deletions generally range from about 1 to 30 residues, more preferably 1 to 10 residues. Amino acid sequence insertions include amino and/or carboxyl terminal fusions from one residue to polypeptides of essentially unrestricted length, as well as intrasequence insertions of single or multiple amino acid residues. Intrasequence insertions, (i.e., insertions within the complete protein sequence) can range generally from about 1 to 10 residues, more preferably 1 to 5.

The third group of variants are those in which at least one amino acid residue in the polypeptide molecule, and preferably, only one, has been removed and a different residue inserted in its place.

Substantial changes in functional or immunological identity are made by selecting substitutions that are less conservative, i.e., selecting residues that differ more significantly in their effect on maintaining a) the structure of the polypeptide backbone in the area of the substitution, for example, as a sheet or helical conformation, b) the charge or hydrophobicity of the molecule at the target site, or c) the bulk of the side chain. A conservative substitution is a substitution in which the substituting amino acid (naturally occurring or modified) is structurally related to the amino acid being substituted, i.e., has about the same size and electronic properties as the amino acid being substituted. Thus, the substituting amino acid would have the same or a similar functional group in the side chain as the original amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Further substitutions may comprise those in which:

a) glycine and/or proline is substituted by another amino acid or is deleted or inserted;

b) a hydrophilic residue, e.g., seryl or threonyl, is substituted for a hydrophobic residue, e.g., leucyl, isoleucyl, phenylalanyl, valyl, or alanyl;

c) a cysteine residue is substituted for any other residue;

d) a residue having an electropositive side chain, e.g., lysyl, arginyl, or histidyl, is substituted for a residue having an electronegative charge, e.g., glutamyl or aspartyl; or

e) a residue having a bulky side chain, e.g., phenylalanine, is substituted for one not having such a side chain, e.g., glycine.

Some deletions, insertions and substitutions are not expected to produce radical changes in the characteristics of the protein. One skilled in the art will appreciate that the effect of the substitutions can be routinely evaluated using the animal models such as the model disclosed herein as well as biochemical and in vivo screening assays.

In one embodiment, the invention relates to methods of using epitopes of the proteins described in Table I to elicits an antibody response. Methods of selecting antigenic epitope fragments are well known in the art (Sutcliffe et al., 1983, Science. 219:660-666). Antigenic epitope-bearing peptides and polypeptides of the invention are useful to raise an immune response that specifically recognizes the polypeptides. Antigenic epitope-bearing peptides and polypeptides of the invention comprise at least 4 amino acids (preferably, 6, 7, 9, 10, 12, 15 or 20 amino acids) of the proteins of the amino acid sequence variants of the proteins listed in Table I can be prepared by mutations in the DNA. Such variants include, for example, deletions from, or insertions or substitutions of, residues within the amino acid sequence shown in Table II. Any combination of deletion, insertion, and substitution can also be made to arrive at the final construct, provided that the final construct possesses the desired activity. In one embodiment, the proteins described herein are used to make antibodies specific to polypeptide sequences corresponding to wild type or altered forms of the protein. Antibodies may also be used as probes or for prophylacetic or therapeutic treatment.

The present invention provides methods to screen for proteins implicated in misfolding and protein aggregation. For example, the sequences listed in Table I were derived from screening an RNAi library using a transgenic nematode line overexpressing a human alpha-synuclein::GFP fusion protein. Other reporter molecules such as GFP, RFP, BFP, YFP and luciferase may also be expressed as fusion proteins with alpha-synuclein. Other aggregation-prone proteins may be over-expressed in this manner to study protein misfolding and aggregation for other neurological diseases such as, but not limited to tau and beta-amyloid protein in Alzheimer's disease, mutant-huntingtin in Huntington's disease, SOD1 and neurofilament in amyotrophic lateral sclerosis, and mutant androgen receptor in spinal and bulbar muscular atrophy. With particular reference to Parkinson's disease, overexpression of alpha-synuclein results in the formation of visual aggregates of alpha-synuclein detectable by fluorescent microscopy in the nematode C. elegans. Gene expression is under the control of the unc-54 promoter to direct expression to the body wall for easy visualization. TOR-2 is a protein that has been shown to reduce protein aggregation in C. elegans overexpressing alpha-synuclein. A transgenic worm line containing alpha-synuclein::GFP+TOR-2 may be used for RNAi screening of candidate genes related to misfolding and protein aggregation. Similar suppression of misfolding and protein aggregation by TOR-2 has been previously reported for polyglutamine-dependent protein aggregation (Caldwell et al. Hum Mol Genet. 2003 Feb. 1; 12(3):307-19). This transgenic organism provides a rapid screening method using RNAi feeding in body-wall muscles of worms containing alpha synuclein::GFP+TOR-2 to find genes that cause a return in alpha-synuclein aggregation RNAi knockdown of gene expression. A library of C. elegans genes may be screened with routine experimentation using RNAi to determine the effect of gene knockdown on the aggregation of alpha-synuclein with reproducible results. Generally, for a target gene to be scored as implicated in protein aggregation, an aggregate phenotype occurs in approximately 80% of the alpha-synuclein::GFP+TOR-2 organisms that are assayed. Homologous sequences may be determined using the NCBI BLAST database. (NCBI, National Library of Medicine, NIH, Bethesda, Md.).

In another embodiment, the genes of the present encode proteins that impart neuroprotective qualities to neurons. According to the teachings herein, the C. elegans gene library can be screened to determine if a candidate gene provides protection for neurons. For example, treatment with the neurotoxin 6-OHDA causes loss of dopaminergic neurons in a C. elegans model. Overexpression of select genes prevents dopaminergic neuron loss caused by 6-OHDA treatment. Treatment with 6-OHDA results in damage and death through the formation of reactive oxygen species. As such, 6-OHDA treatment provides a model to assay neuroprotection for neurological diseases related to the formation of reactive oxygen species.

Likewise, neurological disease models may be produced that express aggregation-prone proteins implicated in neurological diseases. For example, overexpression of human alpha-synuclein in C. elegans dopamine neurons recapitulates the neurodegenerative aspects of Parkinson's disease, as these animals exhibit loss of dopamine neurons over time as they age. (Cao et al., J Neurosci. 2005 Apr. 13; 25(15):3801-12). In this context, transgenic worms represent model systems to identify neuroprotective functions of specific compounds and genes.

C. elegans overexpressing target genes are prepared starting with transgenic worms expressing a fluorescent protein such as GFP, RFP, BFP, luciferase or the like under the control of a neuron specific promoter. Neuron specific promoters are routinely available in the art and include, but are not limited to, the promoters controlling expression of neurotransmitter synthesis enzymes and neurotransmitter transporters for example, tyrosine hydroxylase, dopamine beta hydroxylase, dopamine transporter, serotonin transporter, vesicular acetylcholine transporter and the like.

In another embodiment, the present invention relates to methods of using nucleic acid probes for the specific detection of the presence of related nucleic acids in a sample including DNA or RNA molecules corresponding to the above-described nucleic acid molecules or at least a fragment thereof which hybridizes under stringent hybridization and wash conditions to the nucleic acid.

In certain applications, the detection of the polynucleotides described herein may be incorporated in diagnostic assays to indicate the presence or propensity toward protein misfolding or aggregation associated with neurodegenerative disease. In one preferred embodiment, the present invention relates to an isolated nucleic acid probe consisting of 10 to 1000 nucleotides (preferably, 10 to 500, 10 to 100, 10 to 50, 10 to 35, 20 to 1000, 20 to 500, 20 to 100, 20 to 50, or 20 to 35) which hybridizes preferentially to an RNA or DNA fragment, wherein said nucleic acid probe is or is complementary to a nucleotide sequence consisting of at least 10 consecutive nucleotides (preferably, 15, 18, 20, 25, or 30) from the nucleic acid molecule comprising a polynucleotide sequence at least 90% identical to one or more of the following: a nucleotide sequence encoding a polypeptide from those listed in Table II; a nucleotide sequence complementary to any of the above nucleotide sequences; and any nucleotide sequence as previously described above.

The hybridization probes of the present invention can be labeled for detection by standard labeling techniques such as with a radiolabeling, fluorescent labeling, biotin/avidin labeling, chemiluminescence, and the like. After hybridization, the probes can be visualized using known methods.

In another embodiment, the present invention relates to a method of detecting the presence of a nucleic acid in a sample by contacting the sample with the above-described nucleic acid probe, under specific hybridization conditions such that hybridization occurs, and detecting the presence of the probe bound to the nucleic acid molecule. One skilled in the art would select the nucleic acid probe according to techniques known in the art as described above. Samples to be tested include, but should not be limited to RNA or DNA samples from human tissue.

The test samples suitable for nucleic acid probing methods of the present invention include, for example, cells or nucleic acid extracts of cells, or biological fluids. The sample used in the described methods will vary based on the assay format, the detection method and the nature of the tissues, cells or extracts used in the assay. Methods for preparing nucleic acid extracts of cells are well known in the art and can be readily adapted in order to obtain a sample which is compatible with the method utilized.

Methods are provided for the diagnosis of neurological disease by detecting alterations in a protein related to misfolding/aggregation or that provides neuroprotection. In these methods, a tissue sample from an individual is analyzed for alterations in a protein selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins where the presence of alterations indicates a predisposition to, or the presence of a neurological disease. As used herein, a “tissue’ refers to a biological sample from an individual. Examples of such samples include, but are not limited to a sample of cells, an individual cell, a sample of bodily fluid such as blood, lymph, or saliva where cells may or may not be present in the sample.

In one embodiment, the method entails detecting a protein selected from ubiquitin-proteasome degradation system proteins, autophagy proteins, molecular chaperones, transcription factors, vesicular trafficking proteins, Mn/Fe transporters, HSPC117 proteins, acetylcholine receptor subunits, DJ-1 proteins and PINK-1 proteins in a sample, comprising: contacting the sample with an above-described antibody (or protein), under conditions such that immunocomplexes form, and detecting the presence of the antibody bound to the polypeptide. The antibody or protein that specifically binds may be conjugated to a detectable label. In detail, the methods comprise incubating a test sample with one or more of the antibodies of the present invention and assaying whether the antibody binds to the test sample. Alterations in the levels or activity of a protein in a sample as compared to normal levels can indicate a specific disease.

In a further embodiment, the present invention relates to a method of detecting an antibody specific to a protein from Table I in a sample, comprising: contacting the sample with a protein from Table I, under conditions such that immunocomplexes form, and detecting the presence of the protein bound to the antibody or antibody bound to the protein. In detail, the methods comprise incubating a test sample with one or more of the proteins of the present invention and assaying whether the antibody binds to the test sample.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluorescent assays) can readily be adapted to employ the antibodies of the present invention (Chard, In: An Introduction to Radioimmunoassay and Related Techniques, Elsevier Science Publishers, Amsterdam, The Netherlands (1986); Bullock, et al., In: Techniques in Immunocytochemistry, Academic Press, Orlando, Fla. Vol. 1 (1982), Vol. 2 (1983), Vol. 3 (1985); Tijssen, In: Practice and Theory of enzyme Immunoassays: Laboratory Techniques in Biochemistry and Molecular Biology, Elsevier Science Publishers, Amsterdam, The Netherlands (1985)).

The immunological assay test samples of the present invention include cells, protein or membrane extracts of cells, or biological fluids such as blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can readily be adapted in order to obtain a sample which is capable with the system utilized.

The claimed invention utilizes several suitable assays which can measure proteins that aggregate and cause neurological disease. Suitable assays encompass immunological methods, such as radioimmunoassay, enzyme-linked immunosorbent assays (ELISA), chemiluminescence assays and the like.

In several of the preferred embodiments, immunological techniques detect levels of a protein from Table I by means of an antibody cocktail (i.e., one or more antibodies) which includes monoclonal and/or polyclonal antibodies, and mixtures thereof. For example, these immunological techniques can utilize mixtures of polyclonal and/or monoclonal antibodies, such as a cocktail of murine monoclonal and rabbit polyclonal.

One of skill in the art can raise antibodies against an appropriate immunogen, such as isolated and/or recombinant protein or a portion or fragment thereof (including synthetic molecules, such as synthetic peptides). In one embodiment, antibodies are raised against an isolated and/or recombinant protein from the list in Table I or a portion or fragment thereof (e.g., a peptide) or against a host cell which expresses one of these recombinant proteins. In addition, cells expressing recombinant proteins, such as transfected cells, can be used as immunogens or in a screen for antibodies which bind to the proteins.

According to the method, an assay can determine the level or concentration of protein in a biological sample. In determining the amounts of protein, an assay includes combining the sample to be tested with an antibody having specificity for proteins, under conditions suitable for formation of a complex between antibody and protein, and detecting or measuring (directly or indirectly) the formation of a complex. The sample can be obtained and prepared by a method suitable for the particular sample (e.g., whole blood, tissue extracts, serum) and assay format selected. For example, suitable methods for whole blood collection are venipuncture or obtaining blood from an indwelling arterial line. The container to collect the blood can contain an anti-coagulant such as CACD-A, heparin, or EDTA. Methods of combining sample and antibody, and methods of detecting complex formation are also selected to be compatible with the assay format. Suitable labels can be detected directly, such as radioactive, fluorescent or chemiluminescent labels; or indirectly detected using labels such as enzyme labels and other antigenic or specific binding partners like biotin and colloidal gold. Examples of such labels include fluorescent labels such as fluorescein, rhodamine, CY5, APC, chemiluminescent labels such as luciferase, radioisotope labels such as ³²P, ¹²⁵I, ¹³¹I, enzyme labels such as horseradish peroxidase, and alkaline phosphatase, beta-galactosidase, biotin, avidin, spin labels and the like. The detection of antibodies in a complex can also be done immunologically with a second antibody which is then detected. Conventional methods or other suitable methods can directly or indirectly label an antibody.

In another embodiment, the compounds listed in Table I may be used for diagnostic and screening methods that encompass detecting the presence, or absence of, a mutation in a gene wherein the mutation in the gene results in a neuronal disease in a human. For example, the diagnostic and screening methods of the present invention are especially useful for diagnosing the presence or absence of a mutation or polymorphism in a neuronal gene in a human patient, suspected of being at risk for developing a disease associated with an altered expression level of a protein from Table I based on family history, or a patient in which it is desired to diagnose a disease related to these proteins.

In another embodiment, the polynucleotides described herein can be developed into microarrays for screening for the presence of mutants or the absence of wild-type sequences or sequences that predispose an individual to neurological disorders. Microarrays may comprise wild type or altered sequences described herein to detect alterations in expression of the genes in a tissue sample from an individual. The arrays may include all of the sequence provided herein or fragments and mutants of the sequences that bind with specificity to complementary sequences in the sample. The arrays may also be used to determine increases or decreases in the expression of wild type genes that predispose or indicate the presence of a neurological disorder. In any case, a representative amount of the entire sequence is provided on the array to permit detection of complementary sequences derived from a tissue sample. Arrays or microarrays of polynucleotides are generally nucleic acids such as DNA, RNA, PNA, and cDNA but may also include proteins, polypeptides, oligosaccharides, cells, tissues and any permutations thereof which can specifically bind the target molecules. Screening on a microarray may include the use of detectable labels that are specific to nucleic acid sequences on the array. Such screens may be performed by, for example, spotted microarrays or using the fragment DNA microarray technology of Affymetrix, Inc. (Santa Clara, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). The use of microarray in analyzing gene expression is reviewed generally by Fritz et al Science 288:316, 2000; “Microarray Biochip Technology”, L Shi, www.Gene-Chips.com. Systems and reagents for performing microarray analysis are available commercially from companies such as Affymetrix, Inc., Santa Clara Calif.; Gene Logic Inc., Columbia Md.; HySeq Inc., Sunnyvale Calif.; Molecular Dynamics Inc., Sunnyvale Calif.; Nanogen, San Diego Calif.; and Synteni Inc., Fremont Calif. (acquired by Incyte Genomics, Palo Alto Calif.).

“Microarray” and “array,” as used interchangeably herein, refer to an arrangement of a collection of nucleotide sequences in a centralized location. Arrays can be on a surface, for example, a solid substrate, such as a glass slide, or on a semi-solid substrate, such as nitrocellulose membrane. The nucleotide sequences can be DNA, RNA, or any permutations thereof. As is known in the art, a microarray refers to an assembly of distinct polynucleotides or oligonucleotides immobilized at defined positions on a substrate (surface). Arrays are formed on substrates fabricated with materials such as paper, glass, plastic (e.g., polypropylene, nylon), polyacrylamide, nitrocellulose, silicon, optical fiber, polystyrene, or any other suitable solid or semi-solid support, and configured in a planar (e.g., glass plates, silicon chips) or three-dimensional (e.g., pins, fibers, beads, particles, microtiter wells, capillaries) configuration. Polynucleotides or oligonucleotides forming arrays may be attached to the substrate by any number of ways including (i) in situ synthesis (e.g., high-density oligonucleotide arrays) using photolithographic techniques (see, Fodor et al., Science (1991), 251:767-773; Pease et al., Proc. Natl. Acad. Sci. U.S.A. (1994), 91:5022-5026; Lockhart et al., Nature Biotechnology (1996), 14:1675; U.S. Pat. Nos. 5,578,832; 5,556,752; and 5,510,270); (ii) spotting/printing at medium to low-density (e.g., cDNA probes) on glass, nylon or nitrocellulose (Schena et al, Science (1995), 270:467-470, DeRisi et al, Nature Genetics (1996), 14:457-460; Shalon et al., Genome Res. (1996), 6:639-645; and Schena et al., Proc. Natl. Acad. Sci. U.S.A. (1995), 93:10539-11286); (iii) by masking (Maskos and Southern, Nuc. Acids. Res. (1992), 20:1679-1684) and (iv) by dot-blotting on a nylon or nitrocellulose hybridization membrane (see, e.g., Sambrook et al., Eds., 1989, Molecular Cloning: A Laboratory Manual, 2nd ed., Vol. 1-3, Cold Spring Harbor Laboratory (Cold Spring Harbor, N.Y.)).

In one embodiment, the microarray comprises sequences that related to proteins selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins in the preparation of an array to diagnose a neurological disorder.

In another embodiment, the present invention relates to a method of screening for compounds which stimulate or reduces the activity of a protein from Table I. These proteins may also be expressed in vitro and purified for screening assays or expressed in animal models for protein misfolding/aggregation and neurotoxicity. For random screening, agents such as peptides, carbohydrates, pharmaceutical agents and the like are selected at random and are assayed for their ability to bind to or stimulate/reduce the activity of the protein. Such methods include incubating a cell expressing the protein with a compound to be tested; and assaying the cell for the activity of the protein by measuring the compound's effect on ATP binding of the protein. Any cell may be used in the above assay so long as it expresses a functional form of the protein and protein activity can be measured. The preferred expression cells are eukaryotic cells or organisms. Such cells can be modified to contain DNA sequences encoding the protein using routine procedures known in the art. Alternatively, one skilled in the art can introduce mRNA encoding the protein directly into the cell.

In another embodiment, the present invention relates to a screen for pharmaceuticals (e.g., drugs) which can counteract the expression or aberrant activity of an altered protein. Preferably, a neuronal culture is used for the overexpression of the mutant form of proteins using the vector technology described herein. Changes in neuronal morphology and protein distribution is assessed and a means of quantification is used. This bioassay is then used as a screen for drugs which can ameliorate the phenotype. Using ligands to a protein from Table I (including antagonists and agonists as described above), the present invention further provides a method for modulating the activity of the protein in a cell. In general, agents (antagonists and agonists) which have been identified to block or stimulate the activity of the protein can be formulated so that the compound can be contacted with a cell expressing a protein in vivo. The contacting of such a cell with such a compound results in the in vivo modulation of the activity of the proteins.

Candidate compounds may be selected from conventional classes of therapeutics such as small molecule compounds, peptide compounds, peptide mimetics, antibodies, antibody fragments, antibody derivatives, nucleotide molecules, hormones, and the like.

In one embodiment, candidate small molecule compounds may include topoisomerase II inhibitor bacteria calcium channel blocker transpeptidase inhibitor cyclooxygenase inhibitor folic acid synthesis inhibitor and sodium channel blocker. These molecules prevent protein misfolding and aggregation or provide neuroprotection as disclosed in U.S. provisional patent applications 60/738,761 and 60/749,910 incorporated herein by reference in their entirety.

In one embodiment, the topoisomerase II inhibitors may include but are not limited to lomefloxacin, cinoxacin, amsacrine, etoposide, teniposide, oxolinic acid, nalidixic acid, suramin, merbarone, genistein, epirubicin HCl, ellipticine, doxorubicin, or aurintricarboxylic acid (ATA).

In another embodiment, the bacterial transpeptidase inhibitors may include but are not limited to ampicillin, cloxacillin, piperacillin, amoxicillin, cefadroxil, dicloxyacillin, carbenicillin, penicillin, metampicillin, amoxicillin, or cefoxatin.

In another embodiment, the calcium channel blockers may include but are not limited to nimodipine, diproteverine, verapamil, nitrendipine, diltiazem, mioflazine, loperamide, flunarizine, bepridil, lidoflazine, CERM-196, R 58735, R-56865, ranolazine, nisoldipine, nicardipine, PN200-110, felodipine, amlodipine, R-(−)-202-791, or R-(+) Bay K-8644.

In another embodiment, the cyclooxygenase inhibitors may include but are not limited to naproxen, flufenamic acid, tolfenamic acid, fenbufen, ketoprofen, phenacetin, dipyrone, flurbiprofen, meclofenamide, piroxicam, or indomethacine.

In another embodiment, the folic acid synthesis inhibitors may include but are not limited to sulfamethoxazole, sulfadiazine, sulfadoxine, dapsone, trimethoprim, diaveridine, pyrimethamine, or methotrexate.

In another embodiment, the sodium channel blockers may include but are not limited to lidocaine, dyclonine HCl, mexilitine, phenyloin, ketamine, flecainide, or amantadine.

Other agents screened in the assays can be, but are not limited to, peptides, carbohydrates, vitamin derivatives, or other pharmaceutical agents. These agents can be selected and screened at random, by a rational selection or by design using, for example, protein or ligand modeling techniques (preferably, computer modeling).

The nucleotide sequences and proteins described in Table I may also be used to design new compounds to act as agonists, antagonists or binding partners to an endogenous molecules. Active test agents identified by the screening methods described herein that affect misfolding and protein aggregation can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

Once analogs have been prepared, they can be screened using the methods disclosed herein to identify those analogs that exhibit an increased ability to modulate protein aggregation. Such compounds can then be subjected to further analysis to identify those compounds that have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

Alternatively, agents may be rationally selected or designed. As used herein, an agent is said to be “rationally selected or designed” when the agent is chosen based on the configuration of the protein.

Quantitative Structure-Activity Relationship (QSAR) methods may be used to quantify the relationship between the chemical structure of a compound and its biological activity. Each compound class may be quantified or rated for broad-spectrum efficacy using one or more techniques that include a structure-activity relationship (SAR) and/or a quantitative structure-activity relationship (QSAR) method which identify one or more activity related to one or more structures that are related to the class of compounds. Each of these compound classes may then be prioritized based on such factors as synthesizability, flexibility, patentability, activities, toxicities, and/or metabolism. In this case, all or an additional set of compounds within each particular compound class may be assayed and analyzed. As some compound classes may be very large, a subset of the compounds in the classes may be assayed and analyzed and if the class continues to demonstrate efficacy in excess of a predetermined level, the remaining members will be assayed. This approach will also identify functional analogues of compounds and classes of compounds for use in the present invention. The activity of functional analogues may then be confirmed using the C. elegans model to screen for neuroprotection and actions on protein misfolding and aggregation.

Computer modeling technology allows visualization of the three-dimensional atomic structure of a selected molecule and the rational design of new compounds that will interact with the molecule. These methods provide a way to find functional analogues of known small molecule compounds that are known to have actions on neuroprotection and on protein misfolding and aggregation. Analysis of the three dimensional structure of a compound as it binds to a target protein will identify the site of interaction which is then used to identify similar compounds and functional analogues that would have similar binding properties. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modelling systems are the CHARMm and QUANTA programs, Polygen Corporation, Waltham, Mass. CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modeling of drugs interactive with specific proteins. (Schneider and Fechner, Nat Rev Drug Discov. 2005 August; 4(8):649-63; Guner, IDrugs. 2005 July; 8(7):567-72; and Hanai, Curr Med Chem. 2005; 12(5):501-25.) Other computer programs that screen and graphically depict chemicals are available from companies such as BioDesign, Inc., Pasadena, Calif., and Hypercube, Inc., Cambridge, Ontario. Although these are primarily designed for application to drugs specific to particular proteins, they can be adapted to design of drugs specific to regions of DNA or RNA, once that region is identified. Although described above with reference to design and generation of compounds which could alter binding, one could also screen libraries of known compounds, including natural products or synthetic chemicals, and biologically active materials, including proteins, for compounds which are inhibitors or activators. The activity of compounds identified using this approach may be confirmed using the C. elegans model to screen for neuroprotection actions on protein misfolding and aggregation.

The present invention also provides transgenic animal models for use in screening compounds for prophylacetic and therapeutic application. The transgenic animals of the invention are animals into which has been introduced by non-natural means (i.e. by human manipulation), one or more genes that do not occur naturally in the animal, e.g., foreign genes, genetically engineered endogenous genes, etc. The non-naturally introduced genes, known as transgenes, may be from the same or a different species as the animal but not naturally found in the animal in the configuration and/or at the chromosomal locus conferred by the transgene.

Transgenes may comprise foreign DNA sequences, i.e., sequences not normally found in the genome of the host animal. Alternatively or additionally, transgenes may comprise endogenous DNA sequences that are abnormal in that they have been rearranged or mutated in vitro in order to alter the normal in vivo pattern of expression of the gene, or to alter or eliminate the biological activity of an endogenous gene product encoded by the gene (Watson, J. D., et al., In: Recombinant DNA, 2d Ed., W. H. Freeman & Co., New York (1992), pg. 255-272; Gordon, J. W., 1989, Intl. Rev. Cytol. 115:171-229; Jaenisch, R., 1989, Science. 240:1468-1474; Rossant, J., 1990, Neuron. 2:323-334). Transgenes may be incorporated by pronuclear injection, ES cell transfer, viral integration methods all of which are known to one of ordinary skill in the art.

The non-human animals of the invention comprise any animal having a transgenic interruption or alteration of the endogenous gene(s) (knock-out animals) and/or into the genome of which has been introduced one or more transgenes that direct the expression of a protein selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins.

Such non-human animals include vertebrates such as rodents, non-human primates, sheep, dog, cow, amphibians, reptiles, etc. Preferred non-human animals are selected from non-human mammalian species of animals, most preferably, animals from the rodent family including rats and mice, most preferably mice.

Resultant transgenic non-human animals that are predisposed to a disease, or in which the transgene causes a disease, may be used to identify compositions that induce the disease and to evaluate the pathogenic potential of compositions known or suspected to induce the disease (Bems, A. J. M., U.S. Pat. No. 5,174,986), or to evaluate compositions which may be used to treat the disease or ameliorate the symptoms thereof (Scott, et al., WO 94/12627).

Target genes are chromosomally integrated and overexpress the target protein in these transgenic organisms.

In one embodiment, the present invention provides a transgenic animal that manifests symptoms of protein misfolding and aggregation related neurological disease by expressing defective protein folding machinery or aggregation-prone proteins. Other aggregation-prone proteins such as mutant-huntingtin, beta-amyloid, tau, alpha-synuclein, mutant androgen receptor, mutant SODI, mutant ataxin and the like may be used to model other neurological diseases. As an example, in one embodiment, a transgenic organism is used that overexpresses alpha-synuclein protein in neurons using a neuron specific promoter. Overexpression of alpha-synuclein results in misfolded protein intermediates, protein aggregation and neuronal degeneration. This transgenic line may be crossbred with organisms overexpressing target genes identified from previous RNAi screening to determine if the target gene products confer neuroprotective qualities and reduce the toxic effects of misfolding and aggregation of alpha-synuclein. Other models may be used where the transgene is an altered form of a gene selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins. The alteration may include increased or decreased expression or a mutation or alternately spliced forms of the protein that results in symptoms of a neurological disease.

In a model for assaying neuroprotection, transgenic organisms treated with a neurotoxin such as 6-hydroxydopamine (6-OHDA) which is known to destroy dopamine containing neurons. Other neurotoxins may also be used in this screening method and are known to one of ordinary skill in the art. Neuronal morphology can be routinely screened after toxin exposure with fluorescence microscopy.

For example, this screening method identified one gene product that is characterized by its ability to protect dopaminergic neurons from a 6-OHDA insult. The C. elegans gene is called M7.5 (SEQ ID NO:41) and corresponds to a human E1-like gene. (SEQ ID NO:43). There is a high degree of conservation for this gene with human, worm, bovine, rat, and mouse sequences having an e-value of zero. As such, other species homologues should have the same function on providing neuroprotection. Overexpression of M7.5 confers neuroprotection to dopamine neurons after exposure to the neurotoxin 6-OHDA. Similarly, torsin proteins also confer neuroprotection to dopaminergic neurons after exposure to the neurotoxin 6-OHDA. (Cao et al., J Neurosci. 2005 Apr. 13; 25(15):3801-12). Transgenic worms provide a model system to screen for neuroprotective effects of other genes or compounds.

As used herein, a cell is said to be “altered to express a desired peptide” when the cell, through genetic manipulation, is made to produce a protein which it normally does not produce or which the cell normally produces at low levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression.

The nucleic acid molecules and proteins described herein provide therapeutic targets to treat neurological disease. Neurological diseases caused by deficient or defective genes or proteins may be treated by restoring function of the genes or proteins. Such restoration may be accomplished by using gene therapy, or administering a compound to restore function of the normal gene or protein.

Functional DNA can be provided to the cells of such patient in a manner and amount that permits the expression of the protein encoded by such gene, for a time and in a quantity sufficient to treat such patient afflicted with or predisposed to a neurological disease caused by a deficient or defective protein. Many vector systems are known in the art to provide such delivery to human patients in need of a gene or protein missing from the cell. For example, retrovirus systems can be used, especially modified retrovirus systems and especially herpes simplex virus systems (Breakefield, X. O., et al., 1991, New Biologist. 3:203-218; Huang, Q., et al., 1992, Experimental Neurology. 115:303-316; WO93/03743; WO90/09441). Delivery of a DNA sequence encoding a functional protein will effectively replace the missing or mutated gene causing the disorder.

In another embodiment of this invention, the gene is expressed as a recombinant gene in a cell, so that the cells can be transplanted into a mammal, preferably a human in need of gene therapy. To provide gene therapy to an individual, a genetic sequence which encodes for all or part of the gene is inserted into a vector and introduced into a host cell. In another embodiment, expression of a defective or malfunctioning protein may be reduced using RNAi. Such methods are reviewed in Forte et al. (Curr Drug Targets. 2005 February; 6(1):21-9).

Examples of diseases that can be suitable for gene therapy include, but are not limited to, neurodegenerative diseases or disorders. Such disorders include Parkinson's disease, Alzheimer's disease, prion diseases, polyglutamine disease, tauopathy, Huntington's disease, dystonia, familial amyotrophic lateral sclerosis, Pick's disease, progressive supranuclear palsy and cortical degeneration.

Gene therapy methods can be used to transfer the coding sequence of a protein from Table I to a patient (Chattedee and Wong, 1996, Curr. Top. Microbiol. Immunol. 218:61-73; Zhang, 1996, J. Mol. Med. 74:191-204; Schmidt-Wolf and Schmidt-Wolf, 1995, J. Hematotherapy. 4:551-561; Shaughnessy, et al., 1996, Seminars in Oncology. 23:159-171; Dunbar, 1996, Annu. Rev. Med. 47:11-20).

Examples of vectors that may be used in gene therapy include, but are not limited to, defective retroviral, adenoviral, or other viral vectors (Mulligan, R. C., 1993, Science. 260:926-932). The means by which the vector carrying the gene can be introduced into the cell include but is not limited to, microinjection, electroporation, transduction, or transfection using DEAE-Dextran, lipofection, calcium phosphate or other procedures known to one skilled in the art (Sambrook, J., Fritsch, E. F., and Maniatis, T., 1989, In: Molecular Cloning. A Laboratory Manual., Cold Spring Harbor Laboratory Press, Cold Spring Harbor).

Compounds of the present invention, including therapeutic compounds discovered using the described screening methods, may be administered to treat a neurological disease. In one embodiment a composition is administered comprising a therapeutically effective amount of a compound to treat, reduce or eradicate symptoms of the neurological disease. One skilled in the art will also appreciate that the amounts to be administered for any particular treatment protocol can readily be determined. The dosage should not be so large as to cause adverse side effects, such as unwanted cross-reactions, anaphylactic reactions, and the like. Generally, the dosage will vary with the age, condition, sex and extent of disease in the patient, counter indications, if any, and other such variables, to be adjusted by the individual physician. The dosages used in the present invention to provide immunostimulation include from about 0.1 μg to about 500 μg, which includes, 0.5, 1.0, 1.5, 2.0, 5.0, 10, 15, 20, 25, 30, 35, 40, 45, 50, 55, 60, 65, 70, 75, 80, 85, 90, 95, 100, 150, 200, 250, 300, 350, 400, and 450 μg, inclusive of all ranges and sub-ranges there between. Such amount may be administered as a single dosage or may be administered according to a regimen, including subsequent booster doses, whereby it is effective, e.g., the compositions of the present invention can be administered one time or serially over the course of a period of days, weeks, months and/or years. The dosage may be administered in a pharmaceutically acceptable carrier.

Also, the dosage form such as injectable preparations (solutions, suspensions, emulsions, solids to be dissolved when used, etc.), tablets, capsules, granules, powders, liquids, liposome inclusions, ointments, gels, external powders, sprays, inhalating powders, eye drops, eye ointments, suppositories, pessaries, and the like can be used appropriately depending on the administration method, and the peptide of the present invention can be accordingly formulated. Pharmaceutical formulations are generally to known in the art, and are described, for example, in Chapter 25.2 of Comprehensive Medicinal Chemistry, Volume 5, Editor Hansch et al, Pergamon Press 1990.

A protein from Table I or ligand thereof can be administered parenterally by injection or by gradual perfusion over time. It can be administered intravenously, intraperitoneally, intramuscularly, intrathecally or subcutaneously. Other methods to assure that a compound may cross the blood-brain barrier are also contemplated for use in administering the compound.

Preparations for parenteral administration include sterile or aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present, such as, for example, antimicrobials, antioxidants, chelating agents, inert gases and the like (Remington's Pharmaceutical Science, 16th ed., Eds.: Osol, A., Ed., Mack, Easton Pa. (1980)).

In another embodiment, the present invention relates to a pharmaceutical composition comprising a protein from Table I or ligand thereof in an amount sufficient to alter the activity of the protein, and a pharmaceutically acceptable diluent, carrier, or excipient. Appropriate concentrations and dosage unit sizes can be readily determined by one skilled in the art as described above (Remington's Pharmaceutical Sciences, 16th ed., Eds.: Osol, A., Ed., Mack, Easton Pa. (1980); WO 91/19008).

The pharmaceutically acceptable carrier which can be used in the present invention includes, but is not limited to, an excipient, a binder, a lubricant, a colorant, a disintegrant, a buffer, an isotonic agent, a preservative, an anesthetic, and the like which are commonly used in a medical field.

In another embodiment, the present invention relates to a method of administering a protein from Table I or a ligand of the protein (including antagonists and agonists) to an animal (preferably, a mammal (more preferably, a human)) in an amount sufficient to effect an altered level of the protein in the animal. The administered protein or ligand could specifically affect protein-associated functions. Further, since the proteins of Table I are expressed in brain tissue, administration of the protein or ligand could be used to alter protein levels or function in the brain. Neurological disorders that may be treated with this method include disorders of protein aggregation such as Alzheimer's disease, Parkinson's disease, prion disease, polyglutamine disease, Tauopathy, Huntington's disease, familial amyotrophic lateral sclerosis, Pick's disease, progressive supranuclear palsy and cortical degeneration.

In another embodiment, the present invention relates to a kit for detecting, in a sample, the presence of a nucleic acid or protein listed in Table I. In one embodiment, the kit includes reagents and instructions for their use to detect an altered protein or diagnose the predisposition to or the existence of a neurological disease. The kit may include at least one container having disposed therein the above-described nucleic acid probe. In a preferred embodiment, the kit further comprises other containers comprising wash reagents and/or reagents capable of detecting the presence of the hybridized nucleic acid probe. Examples of detection reagents include, but are not limited to radiolabeled probes, enzymatic probes (horseradish peroxidase, alkaline phosphatase), and affinity labeled probes (biotin, avidin, or streptavidin). In one embodiment the kit comprises one or more reagents for detecting the disorder by carrying out a PCR, hybridization or sequence-based assay or any combination thereof such as a microarray.

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris buffers, and the like), and containers which contain the reagents used to detect the hybridized probe, bound antibody, amplified product, or the like.

One skilled in the art will readily recognize that the nucleic acid probes described in the present invention can readily be incorporated into one of the established kit formats which are well known in the art.

In another embodiment of the present invention, a kit is provided for detecting the presence or absence of a protein listed in Table I; or the likelihood of developing a disorder in a mammal on the basis of the presence of absence of a protein listed in Table I. This particular kit contains all the necessary reagents to carry out the previously described methods of detection.

For example, the kit can comprise a first container means containing an above-described antibody, and a second container means containing a conjugate comprising a binding partner of the antibody and a label.

The kit may also comprise a first container means containing an above-described protein, and preferably a second container means containing a conjugate comprising a binding partner of the protein and a label. More specifically, a diagnostic kit comprises a protein from the list in Table I as described above, to detect antibodies in the serum of potentially infected animals or humans.

In another preferred embodiment, the kit further comprises one or more other containers comprising one or more of the following: wash reagents and reagents capable of detecting the presence of bound antibodies. Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit can be as described above for nucleic acid probe kits. The kit can be, for example, a RIA kit or an ELISA kit.

One skilled in the art will readily recognize that the antibodies described in the present invention can readily be incorporated into one of the established kit formats which are well known in the art.

The invention will be described in greater detail by way of specific examples. The following examples are offered for illustrative purposes, and are intended neither to limit nor define the invention in any manner.

### EXAMPLE 1

### Screening for Genes Regulating Protein Aggregation in Parkinson's Disease RNAi

A transgenic C. elegans line overexpressing alpha-synuclein::GFP was developed and results in the formation of visual aggregates of alpha-synuclein detectable by fluorescent microscopy. Gene expression is under the control of the unc-54 promoter to direct expression to the body wall. Another transgenic worm line containing alpha-synuclein::GFP+TOR-2 was used for RNAi screening of candidate genes related to protein aggregation. The presence of TOR-2 in the alpha-synuclein::GFP+TOR-2 worm prevents the aggregation of alpha-synuclein::GFP fusion protein in body-wall muscle cells resulting in a diffuse fluorescence. Similar suppression of protein aggregation by TOR-2 has been previously reported for polyglutamine-dependent protein aggregation (Caldwell et al. Hum Mol Genet. 2003 Feb. 1; 12(3):307-19). This transgenic organism allows for a rapid screening method using RNAi feeding in body-wall muscles of worms containing alpha synuclein::GFP+TOR-2 to find genes that cause an increase in misfolding and return in alpha-synuclein aggregation when depleted by RNAi.

A library of C. elegans genes was screened using RNAi to determine the effect of gene knockdown on the aggregation of alpha synuclein. This RNAi library of 18,000 bacterial strains was purchased for use in bacterial feeding in genome-wide RNAi screening in C. elegans (Sanger Centre, Cambridge). Rather than conducting a broad screen of the entire C. elegans genome, reasoned targeting of genes implicated in ER-associated degradation (ERAD), ubiquitin proteosome system (UPS), autophagy, Parkinson's disease and interactome and microarray co-expression data identified candidate molecules affecting protein aggregation for screening.

Briefly, fresh cultures of E. coli expressing target gene dsRNA were prepared on LB agar plates containing ampicillin and tetracycline and grown overnight. Fresh cultures of dauer alpha-synuclein::GFP worms and 3 mL bacterial cultures of the E. coli expressing the target gene were prepared the next day. On the day of the experiment, one small and one medium plate per target gene were coated with IPTG and then the bacterial culture allowing for dry time between coating with each material. Five L4 worms were placed on each medium plate for approximately 42 hours at 25 degrees Celsius. All original adult worms were then transferred to the small IPTG/bacteria coated plate for 9 hours after which the original adults were burned off. The offspring were analyzed 36 hours later for expression of a resulting phenotype.

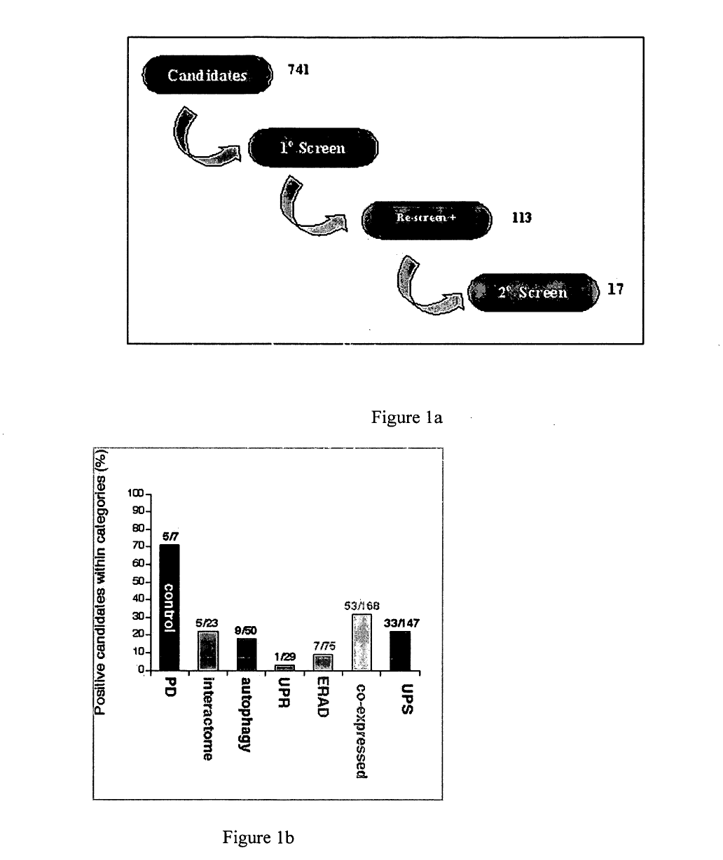

A group of over 741 possible gene targets were chosen based on their relevance to protein aggregation and were subjected to initial RNAi screen. All positive candidate genes were screened a second time to eliminate false positives. The primary and secondary screens resulted in 113 positive genes. The distribution of genes identified in the primary screen these 741 genes using alpha-synuclein::GFP+TOR-2 worms for presence of aggregates is shown in FIG. 1b.

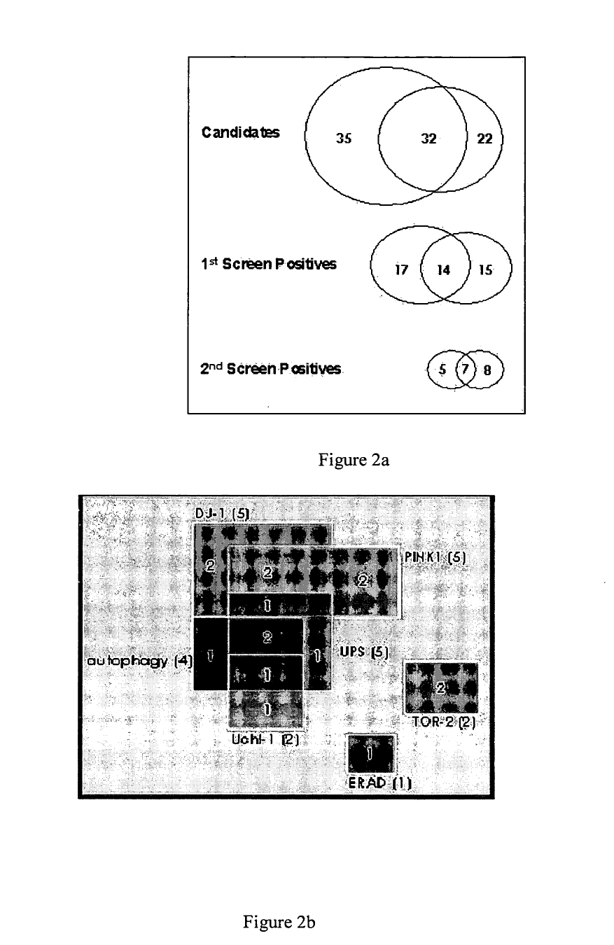

Candidates were also identified from micro-array experiments between DJ-1 and PINK1 at all stages of the screening process. A sample of 89 candidates were selected that co-expressed with known Parkinson's disease genes DJ-1 and PINK1. The overlap of candidate molecules is shown in FIG. 2a. After two rounds of screening with RNAi, seven positive candidates from the original set of 89 genes alter alpha-synuclein aggregation and are co-expressed with both DJ-1 and PINK1. Interestingly, 2/7 positive overlapping genes encode hypothetical proteins.

Multiple rounds of RNAi analysis (50 worms per gene; 2 repetitions; positives judges as >80% of worms exhibiting increased aggregation) and secondary, more stringent, screening in developmentally-staged animals to identify candidates exhibiting stronger effects over aging have identified 17 candidate genes listed in Table I that reproducibly induce misfolding of human alpha-synuclein when knocked down. These genes are C. elegans homologs of DJ-1, PINK1 and torsinA; 4 UPS components (1 E1 ligase, 3 E3 ligases), 4 components of the autophagy machinery, 1 predicted chaperone, 1 transcription factor, 1 gene product involved in vesicular trafficking and 3 hypothetical proteins of previously unknown function.

Variable phenotypes results after screening alpha-synuclein::GFP+TOR-2 worms for a return to the aggregated states following systematic RNAi knockdown of candidates. These phenotypes included the occasional clustering of aggregates around nuclei.

The findings of these experiments provide a reliable method of screening for proteins implicated in aggregation of alpha-synuclein with routine experimentation. The results of these experiments provide the identities of target proteins to study mutations within them that cause a pathological phenotype while also providing protein targets for rational drug design.

### EXAMPLE 2

### Neuroprotection of Dopamine Neurons by Candidate Gene Expression after 6-OHDA Exposure

C. elegans has precisely 8 dopaminergic neurons that undergo a readily discernable pattern of degeneration upon treatment with 6-hydroxydopamine (6-OHDA), a dopamine analog and neurotoxin which results in the formation of reactive oxygen species in those neurons. Overexpression of either human torsinA or C. elegans TOR-2 in dopamine neurons is able to dramatically suppress neuronal degeneration following alpha-synuclein overexpression or 6-OHDA treatment (Cao et al, J Neurosci. 2005).

The data from the protein aggregation screens were used to prioritize subsequent tertiary screening for the potential activity of these genes in dopaminergic neuroprotection. Transgenic worms expressing GFP within dopamine neurons have been constructed and extensively analyzed (Nass et al 2002; Cao et al 2005) following 6-OHDA exposure. Phenotypic changes are apparent within 2 hours and typically 6 hours following exposure, most dopamine neurons have completely degenerated. The cDNAs encoding candidate proteins from the RNAi screen were cloned under the control of the dopamine promoter (dat-1) to determine if any of these candidate Parkinson's disease-related genes exhibit neuroprotective activity when expressed in dopamine neurons. Wild type cDNAs of candidates were cloned into a dat-1 promoter vector, injected into the worms and assayed for neuron protection following exposure to 6-OHDA. Select candidate genes are then used in screens of Parkinson's patient genomic DNA.

Furthermore, a new isogenic line of nematodes was designed specifically for screening candidate PD genes for evidence of neuroprotection. This new isogenic line contains a chromosomally integrated transgene overexpressing human alpha-synuclein in dopamine neurons alone with GFP to evaluate neurodegeneration in vivo during development and aging. This line exhibits approximately 30-40% degeneration at the 4-day adult stage of C. elegans development and represents an ideal tool for investigation of environmental/genetic factors in which alpha-synuclein predisposition may impact dopamine neurodegeneration. Systematic evaluation of the positive RNAi screen candidates were performed by crossing animals overexpressing corresponding cDNAs in dopamine neurons of this alpha-synuclein strain and then finding evidence of neuroprotection. This strain may also be used in medium through-put screening for small molecule inhibitors of alpha-synuclein dependent degradation.

Materials and Methods

C. elegans Strains and Protocols

Nematodes were maintained using standard procedures (Brenner, 1974). Transgenic lines were generated by transforming P_{dat-1}::GFP with either P_{dat-1}::M7.5 [strain UA38 (baEx38)] or P_{dat-1}::torsinA and P_{dat-1}::TOR-2 into wild type C. elegans (N2 Bristol variety). For the construction of α-synuclein overexpressing lines, P_{dat-1}::GFP and P_{dat-1}:: α-synuclein [strain UA18 (baEx18)] were injected into N2 worms. For each combination of plasmid constructs, multiple worm lines expressing stable extrachromosomal arrays were compared and three representative lines were used for experimental analysis, except for the 6-OHDA experiments, where single representative transgenic lines were used in repeated experiments after initial analysis on all stable lines.

Plasmid Constructs and Mutagenesis

Plasmids were constructed using Gateway™ technology (Invitrogen, Carlsbad, Calif.). Specifically, the unc-54 promoter region was excluded from pPD30.38 (a gift from Andrew Fire) by double digestion using HindIII and KpnI and replaced by the dat-1 promoter region fragment amplified from pRN200 (Nass et al., 2002). The resulting novel vector was then converted into a Gateway™ destination vector, pDEST-DAT-1, using Gateway™ technology. The human α-synuclein cDNA plasmid was obtained from Philipp Kahle. Gateway™ entry vectors were generated by BP reaction with pDONR201 or pDONR221 using PCR amplified cDNA fragments encoding M7.5 (SEQ ID NO: 41) α-synuclein, GFP. Following this, all genes were cloned into the pDEST-DAT-1 vector via an LR reaction with the respective entry vector.

Preparation of C. elegans Extracts for Immunoblotting

Extracts were prepared following growth of each transgenic line to near confluence on two 100 mm NGM plates. Worms were collected by washing with M9 buffer and concentrated by centrifugation in a 1.5 ml microcentrifuge tube at 5,000×g for 1 min. The worm pellet was resuspended and lysed in 0.5 ml worm lysis buffer (100 mM Tris, pH 6.8, 2% SDS, 15% glycerol) by boiling for 5 min. This lysate was centrifuged again at 13,200×g for 10 min and the supernatant was collected then concentrated using a Centricon YM-10 column (Millipore) at 14,000×g for 30 min. Protein concentration was determined using the bicinchoninic acid protein assay (Sigma, St. Louis, Mo.).

SDS-PAGE and Western Blotting

SDS-PAGE and Western blotting were performed as previously described (Caldwell et al., 2003), unless indicated otherwise. For TOR-2 detection, a 1:800 dilution of rabbit-anti-TOR-2 primary antibody (Caldwell et al., 2003) and a 1:10,000 dilution of horseradish peroxidase-conjugated goat-anti-rabbit IgG secondary antibody (Amersham-Pharmacia, Piscataway, N.J.) were used. For actin detection, a 1:8,000 dilution of mouse-anti-actin antibody (ICN) and a 1:10,000 dilution of horseradish peroxidase-conjugated goat-antimouse IgG secondary antibody (Amersham-Pharmacia) were used. For GFP detection, 140 μg of total protein was loaded and a 1:1000 dilution of rabbit-anti-GFP primary antibody (Clontech, Palo Alto, Calif.) and a 1:10,000 dilution of horseradish peroxidase-conjugated goat-anti-rabbit IgG secondary antibody (Amersham-Pharmacia) were used.

6-OHDA Exposure and Quantitative Analyses of Neuronal Degeneration

Age-synchronized worms were obtained by treating gravid adults with 2% sodium hypochlorite, 0.5M NaOH to isolate embryos (Lewis and Fleming, 1995). These embryos were grown for 30 hours at 25° C. At the L3-stage, larvae were incubated with 10 mM (or 50 mM) 6-OHDA and 2 mM (or 10 mM) ascorbic acid for 1 hour with gentle agitation every 10 minutes (Nass et al., 2002). The worms were then washed and spread onto NGM plates seeded with bacteria (OP50) and scored at time points ranging from 2 to 72 hours post-6-OHDA exposure.

Immediately after 6-OHDA treatment, worms containing the non-integrated transgenes were selected under a fluorescence dissecting microscope, based on the presence of GFP, and transferred to a fresh NGM plate seeded with OP50. For each time point, 30-40 worms were applied to a 2% agarose pad and immobilized with 3 mM levamisole. Worms were examined under a Nikon Eclipse E800 epifluorescence microscope equipped with an Endow GFP HYQ filter cube (Chroma). For the ease of analysis, only the four CEP dopaminergic neurons in the head of the worm were scored. A worm was scored as “wild type” when all four CEP neurons were present and their neuronal processes were intact; a worm was scored as having “dendrite blebbing”, “cell body rounding”, or “cell body loss” when at least one of the four neuronal dendrites or cell bodies was defective as described. These experiments were repeated 3 times. Images were captured with a Cool Snap HQ CCD camera (Photometrics) driven by MetaMorph Software (Universal Imaging).

Alpha-Synuclein or CAT-2-Induced Neurodegeneration Analyses

To obtain 7-day-old animals of α-synuclein and CAT-2 transgenic lines, non-integrated L1 and L2 worms with green fluorescence were selected and allowed to grow to the 4 day adult stage (approximately 7 days post hatching). 30-40 worms at each chosen stage were analyzed for each non-integrated line and the average of at least 3 stable lines for each combination of transgenes was reported. A worm was scored as wild type when it still preserved all four CEP cell bodies regardless of the morphology of the dendrites.

Results

Wild-type cDNAs from the candidates have been cloned into a dopamine expression vector for evaluation in neuroprotection assays within transgenic C. elegans. This screening approach has been validated by the demonstration that an autophagy-related gene product increases alpha-synuclein misfolding when knocked down with RNAi as well as the torsin A homolog, TOR-2 both showed dramatic neuroprotection from 6-OHDA exposure when specifically overexpressed in C. elegans dopamine neurons. The overexpression of wild type torsins (P_{dat-1}::torsinA and P_{dat-1}::TOR-2) in DA neurons significantly elevated the resistance of DA neurons to 6-OHDA at a concentration of 10 mM for at least 72 hours compared to control worms.

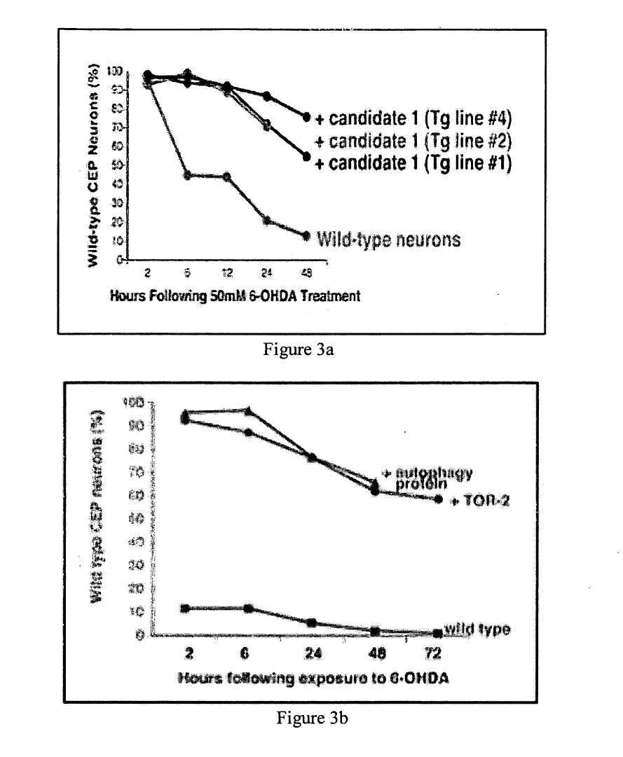

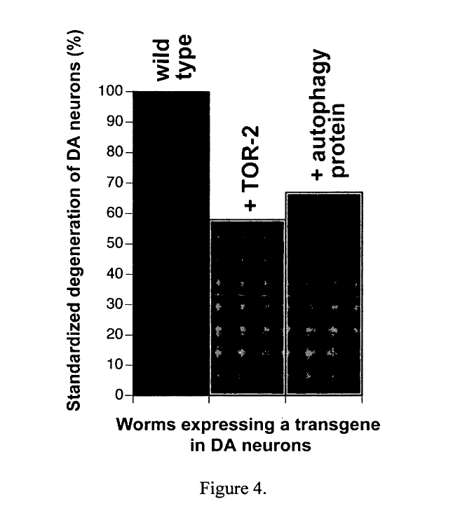

This screening method also identified a gene that is characterized by its ability to protect dopaminergic neurons from a 6-OHDA insult. The C. elegans gene is called M7.5 (SEQ ID NO:41) and corresponds to a human E1-like gene. (SEQ IS NO:43). This gene is a member of the family of ubiquitin activating E1 enzyme-like proteins and functions in autophagy. The M7.5 cDNA was overexpressed in GFP-labeled dopamine neurons and assayed for neuroprotection following exposure to 6-OHDA, Three independent M7.5 expressing transgenic lines were obtained. All three of these lines exhibited dramatic protection of the dopamine neurons from 6-OHDA-induced oxidative stress. The actions of this protein on neuroprotection in DA neurons is shown in FIG. 3. Further studies will differentiate between other candidates that show aggregates early in development and those that only have aggregates as the animals age.

### EXAMPLE 3

### Method of Using a Microarray to Detect Protein Alterations and Diagnose Predisposition to or Presence of Parkinson's Disease in Humans

Production of a Parkinson's Disease Microarray

A Parkinson's Disease microarray is made using standard commercially available microarray technology such as spotted microarrays or the high-density, oligonucleotide-based platform used by Affymetrix, Inc. A moderate to large number of genes and/or transcripts is selected for analysis, i.e., expression (or response) profiling. Nucleic acid sequences that can be monitored in the methods of the present invention include, but are not limited to, those listed with the National Center for Biotechnology Information (on the world wide web at ncbi.nlm.nih.gov) in the GenBank® databases, and sequences provided by other public or commercially-available databases (for example, the NCBI EST sequence database, the EMBL Nucleotide Sequence Database; Incyte's (Palo Alto, Calif.) LifeSeq™ database, and Celera's (Rockville, Md.) “Discovery System”™ database). The present microarrays also include transcripts corresponding to sequences encoding human homologues of proteins from Table I. The array may include the whole sequence corresponding to the gene/transcript or a fragment or fragments of the whole sequence that provides enough specificity to permit detection of a gene/transcript in a sample. Included on the microarray are transcripts or fragments corresponding to SEQ ID NOs: 3, 7, 11, 15, 19, 23, 27, 31, 35, 39, 43, 47, 51, 55, 59, 63, or 67 and combinations thereof including mutant forms and splice variants of these sequences. Other sequences are included on the array comprising other known genes related to Parkinson's disease. Sequences of genes related to oxidative stress and protein dysfunction are also included on the microarray because these processes are known play a role in Parkinson's disease. (Miller et al., Neuroscientist. 2005 December; 11(6):539-49). Other genes associated with Parkinson's disease such as SNPs may also be included on the array.

(Maraganore et al., Am J Hum Genet. 2005 November; 77(5):685-93). The arrays also include positive controls and negative controls.

Use of the Parkinson's Disease Microarray

A tissue sample from an individual such as a biopsy is harvested from the individual and using standard methods to prepare microarray probes, the sample is converted into labeled polynucleotide probes and hybridized to the array and unbound probe is washed off. The array is then scanned using conventional array scanners to detect the label and determine the presence or absence of wild type or mutant forms of genes (qualitative changes) as well as changes in the expression levels (quantitiative changes) of the genes in the patient sample. Standard commercially available data mining software is used to analyze and cluster genetic profiles.

The results from using the microarrays are useful for applications in pharmacogenomics and predictive medicine. Genetic profiles of multiple patients are correlated with the degree of symptoms, onset and severity of the disease to compile a database of Parkinson's disease profiles. Patient profiles are also correlated to patient response to existing treatment methods such as L-DOPA therapy. Efficacy of novel therapeutic compounds is also correlated to patient profiles during early clinical trials to determine optimal genetic profile for a novel treatment.

We claim:

1. A method for detecting alterations in a first protein comprising screening for misfolding or aggregation of at least one second protein,
wherein the first protein is selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins.

2. The method of claim 1 wherein the alteration comprises increased or decreased expression of the first protein.

3. The method of claim 1 wherein the alteration comprises a mutation in the first protein.

4. A method for diagnosing a neurological disease comprising detecting alterations in a protein selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins in a tissue sample from an individual,
wherein alterations indicate a predisposition to or presence of a neurological disease.

5. The method of claim 4 further comprising determining the amount of protein misfolding or aggregation in an in vivo or in vitro model.

6. The method of claim 4 wherein the protein is detected with antibodies detectable labels, nucleic acid probes or microarrays specific to polynucleotide or polypeptide sequences corresponding to wild type or altered forms of the protein.

7. A method of screening for compounds to treat a neurological disease comprising contacting a target compound with a protein selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins, and
determining a change in the activity of the protein in the absence of the compound.

8. The method of claim 7 further comprising administering the compound to an animal model of neurological disease to reduce misfolding and aggregation of at least one second protein or provide neuroprotection.

9. The method of claim 8 wherein the compound is selected from topoisomerase II inhibitors, bacterial transpeptidase inhibitors, calcium channel antagonists, cyclooxygenase inhibitors, folic acid synthesis inhibitors, and sodium channel blockers.

10. A method for treating a neurological disease comprising altering the activity of a first protein selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins in an individual in need of treatment.

11. The method of claim 10 wherein the activity of the first protein is altered by administering a vector expressing a second protein selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins to an individual in need of treatment.

12. The method of claim 10 wherein the protein protects neurons from degeneration and death.

13. The method of claim 10 wherein the activity of the first protein is altered by administering a compound to alter the activity of the first protein in the absence of the compound.

14. The method of claim 10 wherein the neurological disease is selected from amyotrophic lateral sclerosis, Alzheimer's disease, Parkinson's disease, prion disease, polyglutamine expansion diseases, spincocerebellar ataxia, spinal & bulbar muscular atrophy, spongiform encephalopathy, tauopathy, Huntington's disease, or dystonia.

15. The method of claim 10 wherein the activity of the first protein is altered prior to onset of symptoms in an individual predisposed to the neurological disease.

16. The method of claim 13 wherein the compound is selected from topoisomerase II inhibitors, bacterial transpeptidase inhibitors, calcium channel antagonists, cyclooxygenase inhibitors, folic acid synthesis inhibitors, and sodium channel blockers.

17. The method of claim 13 wherein the compound is administered by inhalation, transdermal, oral, rectal, transmucosal, intestinal or parenteral routes in a pharmaceutically acceptable carrier.

18. The method of claim 13 wherein the compound is administered prior to onset of symptoms in an individual predisposed to the neurological disease.

19. A transgenic animal comprising a protein selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins with altered activity than in wild-type animals.

20. The transgenic animal of claim 19 wherein the altered activity comprises increased or decreased expression of the protein or a mutation in the sequence of the protein.

21. A kit for the detection of an altered protein or diagnosis or a neurological disease comprising reagents and instructions on their use to detect the altered protein or diagnose the neurological disease,
wherein the protein is selected from ubiquitin-proteasome degradation system proteins, autophagy proteins, molecular chaperones, transcription factors, vesicular trafficking proteins, Mn²⁺/Fe²⁺ transporters, HSPC117 proteins, acetylcholine receptor subunits, DJ-1 proteins and PINK-1 proteins.


SEQUENCE LISTING (78 sequences)

SEQ ID NO: 1 - DNA, 1239 bp, Caenorhabditis elegans
  CDS (1)..(1239)
atgaaaaagttcgctgaaaaatggtttctattgaaatttaaattctatgttcaatgtttctttatcttcaaatttcgttatcagtgcatcaatctatttttcggtgtgatttctcatggatattttgatgttagcaagaatacgcagataacaagcgacatcttctgttccatttcattttcctttacttctcacttgtcaaatattttgttttattctgaaagaaagatgcaattttttaaatatattattttcgttatcattcttaatcaattagtcgtcgatgtccacagcttatcaatgccaatgtttttaaaatgtttattttacacttgctgcggtgaaacggatatattcaattatcatgcgttatacaaagatttcgataataaaattttcgggcagcacttgatggcagaatctgtagttcattcaatcaaatctcattggcacaatgagcattctcagaagccgctagttctctcatttcacggcggaaccggcactggaaagaattatgtgactgaaattattgtgaacaatacttatcgaagtggaatgcacagcccatttgtgaattatttcgttgcaacaaataattttccgaataaaaagtatattgaggattataaattggaactgaaagatcaacttataagatcggcccgaagatgtcagcgatctatttttatatttgatgagacggataagctacaaagtgaattgattcaagtgatcaaaccatttcttgattattatccggcggtctttggagtggactttcggaaaactatcttcatttttctaagcaacaaagggagcaaagaaattgctaatatcgcattagaacatcatgaaaatggtaaaataagatcacaactcgagttgaagcattttgaacgaacactgatgctttctgcattcaatgaagaaggtggtcttcgtaacactgatatgatctctaatcaacttattgatcattttataccatttcttcccttatctaagttctacgtttcccagtgcattcaagtacatcttcgaaaacgcggaagacatgatttggcaaaggatggagaattcatgcaaagagttcttgattctcttgaatttttccctgaatctagcaaaatattttcctcgtcaggatgtaaacgtgtgaatgcaaagactgatctcgaaatttccaagatgggattctcactcaattcgaagaaagagtttaatgatgagttgtga

SEQ ID NO: 2 - protein, 412 aa, Caenorhabditis elegans
MKKFAEKWFLLKFKFYVQCFFIFKFRYQCINLFFGVISHGYFDVSKNTQITSDIFCSISFSFTSHLSNILFYSERKMQFFKYIIFVIILNQLVVDVHSLSMPMFLKCLFYTCCGETDIFNYHALYKDFDNKIFGQHLMAESVVHSIKSHWHNEHSQKPLVLSFHGGTGTGKNYVTEIIVNNTYRSGMHSPFVNYFVATNNFPNKKYIEDYKLELKDQLIRSARRCQRSIFIFDETDKLQSELIQVIKPFLDYYPAVFGVDFRKTIFIFLSNKGSKEIANIALEHHENGKIRSQLELKHFERTLMLSAFNEEGGLRNTDMISNQLIDHFIPFLPLSKFYVSQCIQVHLRKRGRHDLAKDGEFMQRVLDSLEFFPESSKIFSSSGCKRVNAKTDLEISKMGFSLNSKKEFNDEL

SEQ ID NO: 3 - DNA, 999 bp, Homo sapiens
  CDS (1)..(999)
atgaagctgggccgggccgtgctgggcctgctgctgctggcgccgtccgtggtgcaggcggtggagcccatcagcctgggactggccctggccggcgtcctcaccggctacatctacccgcgtctctactgcctcttcgccgagtgctgcgggcagaagcggagccttagccgggaggcactgcagaaggatctggacgacaacctctttggacagcatcttgcaaagaaaatcatcttaaatgccgtgtttggtttcataaacaacccaaagcccaagaaacctctcacgctctccctgcacgggtggacaggcaccggcaaaaatttcgtcagcaagatcatcgcagagaatatttacgagggtggtctgaacagtgactatgtccacctgtttgtggccacattgcactttccacatgcttcaaacatcaccttgtacaaggatcagttacagttgtggattcgaggcaacgtgagtgcctgtgcgaggtccatcttcatatttgatgaaatggataagatgcatgcaggcctcatagatgccatcaagcctttcctcgactattatgacctggtggatggggtctcctaccagaaagccatgttcatatttctcagcaatgctggagcagaaaggatcacagatgtggctttggatttctggaggagtggaaagcagagggaagacatcaagctcaaagacattgaacacgcgttgtctgtgtcggttttcaataacaagaacagtggcttctggcacagcagcttaattgaccggaacctcattgattattttgttcccttcctccccctggaatacaaacacctaaaaatgtgtatccgagtggaaatgcagtcccgaggctatgaaattgatgaagacattgtaagcagagtggctgaggagatgacatttttccccaaagaggagagagttttctcagataaaggctgcaaaacggtgttcaccaagttagattattactacgatgattga

SEQ ID NO: 4 - protein, 332 aa, Homo sapiens
MKLGRAVLGLLLLAPSVVQAVEPISLGLALAGVLTGYIYPRLYCLFAECCGQKRSLSREALQKDLDDNLFGQHLAKKIILNAVFGFINNPKPKKPLTLSLHGWTGTGKNFVSKIIAENIYEGGLNSDYVHLFVATLHFPHASNITLYKDQLQLWIRGNVSACARSIFIFDEMDKMHAGLIDAIKPFLDYYDLVDGVSYQKAMFIFLSNAGAERITDVALDFWRSGKQREDIKLKDIEHALSVSVFNNKNSGFWHSSLIDRNLIDYFVPFLPLEYKHLKMCIRVEMQSRGYEIDEDIVSRVAEEMTFFPKEERVFSDKGCKTVFTKLDYYYDD

SEQ ID NO: 5 - DNA, 612 bp, Caenorhabditis elegans
  CDS (1)..(612)
atgctcaaatttgtcatagtttcatcaattcttgtggccctaggtctgtcaatcgaattgacattcgaacttccagacaacgcaaatcagtgtttttatgaagatctgaaaaaggatgttgacacagtgtttgaattccaagttgttactggaggccattatgacgtagacttgatcattgaggatccaaatggaaaagttttgtataaagatactaaaaagcagtacgacagtatcaacttcaaggccgaagttgaaggaacatacaaagcatgcttttcaaatgaattctccacattctctcataaaatcgtttacatggattggcaattcggtgatcaaaatgctcttcatgctgccgttactcaaggagctcatgcaatgactcaattagaaaattatgcagtcgctattggagataaattgagaacaattgatgactatcaaactcatcatcgtctccgtgaagcaactggtcgcaaacgtgcagaagaactcaacgagcgcgtgatgatctggtcccttggtcaatctgccgtcgtagtattcattggaattggccaagttttcctgctcaaatcatttttcaatgataaaagaactcgttattaa

SEQ ID NO: 6 - protein, 203 aa, Caenorhabditis elegans
MLKFVIVSSILVALGLSIELTFELPDNANQCFYEDLKKDVDTVFEFQVVTGGHYDVDLIIEDPNGKVLYKDTKKQYDSINFKAEVEGTYKACFSNEFSTFSHKIVYMDWQFGDQNALHAAVTQGAHAMTQLENYAVAIGDKLRTIDDYQTHHRLREATGRKRAEELNERVMIWSLGQSAVVVFIGIGQVFLLKSFFNDKRTRY

SEQ ID NO: 7 - DNA, 675 bp, Homo sapiens
  CDS (1)..(675)
atgccgcggccggggtccgcgcagcgctgggcggccgtcgcgggccgttgggggtgcaggctgctcgcactgctgctactggtgcctggacccggcggcgcctctgagatcaccttcgagcttcctgacaacgccaagcagtgcttctacgaggacatcgctcagggcaccaagtgcaccctggagttccaggtgattactggtggtcactatgatgtagattgtcgattagaagatcctgatggtaaagtgttatacaaagagatgaagaaacagtatgatagttttaccttcacagcctccaaaaatgggacatacaaattttgcttcagcaatgaattttctactttcacacataaaactgtatattttgattttcaagttggagaagacccacctttgtttcctagtgagaaccgagtcagtgctcttacccagatggaatctgcctgtgtttcaattcacgaagctctgaagtctgtcatcgattatcagactcatttccgtttaagagaagctcaaggccgaagccgagcagaggatctaaatacaagagtggcctattggtcagtaggagaagccctcattcttctggtggttagcatagggcaggtatttcttttgaaaagctttttctcagataaaagaaccaccacaactcgtgttggatcataa

SEQ ID NO: 8 - protein, 224 aa, Homo sapiens
MPRPGSAQRWAAVAGRWGCRLLALLLLVPGPGGASEITFELPDNAKQCFYEDIAQGTKCTLEFQVITGGHYDVDCRLEDPDGKVLYKEMKKQYDSFTFTASKNGTYKFCFSNEFSTFTHKTVYFDFQVGEDPPLFPSENRVSALTQMESACVSIHEALKSVIDYQTHFRLREAQGRSRAEDLNTRVAYWSVGEALILLVVSIGQVFLLKSFFSDKRTTTTRVGS

SEQ ID NO: 9 - DNA, 1008 bp, Caenorhabditis elegans
  CDS (1)..(1008)
atggggaagttagaagacgtggaagctgaaaagaaattatgggagagcgacgatgcgtgggagctccgaaaagcattcatgctggcacattatgatgactatccgaaaatccaacttcaatgcctctcacaattattcattaatgtaacacttctcggatgtgaatattctcagactttaatgcaaaagattcgaacaatgggtgctggaattgctgctaacaaagatcgtacaaaaactggaagttacgtgaaggcatctgcagcgaaaaagcgacaagcagtgaagacatcagatttagaaggagcttctgatgagtctaaaaaagtaaaaatggaaaagtctccatcgccagtagctcgtgaatcttttgatgaacgtcttggaaaactcaaggcatctcttgcaatgacacctcaccatctaacgggcgaacaaatgatgaaaactgccacgaatagttgtctcatgaagtggcacgtgaacaagattaaccagaaaattgaaataactatcgatcgatatgtggccttcagacatacattctcccaatactgtgttgatccaagggattgtgcaatcaatactctaattgaaagcattctttcgtgtgacgccgctgttcacgaagaaagctacgaaattagatttgatggagttccggtggacgagtgttacgctaaatcagtcaccagaagacttgcaaaaattaaatcagcagtttctaatggtgcacatacagttaaaggtcttacaacatatttggatgcagttaacatgtcaatgattcaaaatactcaaaaattggaaggatggtctcaacaacttgatctcgtcactgccgatcttcttctatcttctcgtgttttatccagcactgaatgcactaaaccagcgatggcaacgatcgcgaatcaaatgagcgaagacgtgtgtcaactgattctcaacgataaaatcaacgtcattaactctatgaaatctcacagctctcttgcattccaataa

SEQ ID NO: 10 - protein, 335 aa, Caenorhabditis elegans
MGKLEDVEAEKKLWESDDAWELRKAFMLAHYDDYPKIQLQCLSQLFINVTLLGCEYSQTLMQKIRTMGAGIAANKDRTKTGSYVKASAAKKRQAVKTSDLEGASDESKKVKMEKSPSPVARESFDERLGKLKASLAMTPHHLTGEQMMKTATNSCLMKWHVNKINQKIEITIDRYVAFRHTFSQYCVDPRDCAINTLIESILSCDAAVHEESYEIRFDGVPVDECYAKSVTRRLAKIKSAVSNGAHTVKGLTTYLDAVNMSMIQNTQKLEGWSQQLDLVTADLLLSSRVLSSTECTKPAMATIANQMSEDVCQLILNDKINVINSMKSHSSLAFQ

SEQ ID NO: 11 - DNA, 351 bp, Homo sapiens
  CDS (1)..(351)
atggtcggtggcgaggcggctgccgcagtggaggagctggtttcgggggtgcggcaggcggccgacttcgcggagcagttccgctcctactcagagagcgagaagcaatggaaggcccgcatggaattcatcctgcgccacctgcccgactaccgcgacccgcccgacggcagtggccgcctggaccagctgctctccctctccatggtctgggccaaccatctcttcctaggctgcagttacaataaagaccttttagacaaggtgatggaaatggccgatgggattgaagtggaagacctgccacaatttactaccagaagtgaattaatgaaaaagcatcaaagctaa

SEQ ID NO: 12 - protein, 116 aa, Homo sapiens
MVGGEAAAAVEELVSGVRQAADFAEQFRSYSESEKQWKARMEFILRHLPDYRDPPDGSGRLDQLLSLSMVWANHLFLGCSYNKDLLDKVMEMADGIEVEDLPQFTTRSELMKKHQS

SEQ ID NO: 13 - DNA, 1518 bp, Caenorhabditis elegans
  CDS (1)..(1518)
atgcctcgcacatttgaagaagaatgtgattttatcgatcgcttgacagacacaaagtttcgaattaagaaaggttttgtgccgaatatgaatgtggagggacgattttatgtgaataatagtctcgaacaattaatgtttgacgagttaaagttctcatgtgatggacaaggaatcggtggatttttgccagcggtcagacaaattgccaacgtggcatccctaccaggaattgttggacattcgattggtcttccagatattcactctggttatggtttttccattggaaacatcgctgcgttcgacgttggaaatccagaatctgtaatctcacccggaggcgtcggttttgatatcaactgtggagtacgattacttcgaacgaatctttttgaagaaaatgtaaagccattaaaagaacaactaacacaatcactttttgatcatattccagttggagttggttcacgtggtgcaattccaatgcttgcatcagatcttgttgaatgtttagaaatgggaatggattggacattacgtgaaggttattcatgggcagaagataaagaacattgtgaagaatatggacgaatgttacaagcagatgcatcgaaagtttcattgagagctaaaaaacgtggacttccacaattgggaactcttggagcaggaaatcattacgcagaagttcaagttgtcgatgagatttatgataaacatgctgcaagtacaatgggaattgatgaagaagggcaagttgttgtaatgcttcattgtggaagtcgaggacttggacatcaagttgcaactgactcattagttgaaatggaaaaagcaatggctagagatggaattgttgtaaatgataagcagcttgcgtgtgcaagaattaattcggttgaaggcaaaaactacttttccggaatggcggctgctgcaaacttcgcctgggttaacagatcgtgtatcacattctgtgttcgtaatgcattccaaaagacatttggaatgtcagcagatgatatggatatgcaagtaatctatgacgtgtcacataatgtagcaaaaatggaagagcatatggttgatggtagaccacgtcagctgtgtgttcatcgaaaaggagctacacgcgcttttccggctcatcatccattaataccagttgattatcagttaattggacaacctgtactaattggtggaagtatgggaacttgtagttatgttctaacaggaactgaacaaggattagtggaaacttttggaacaacatgtcatggagctggacgagcactttcacgtgccaaatcacgccgaactatcacttgggattcggtaattgatgatttgaaaaagaaggagatctcaattcgcattgcatcgcctaaattgattatggaagaagcacccgaatcgtataagaatgtgacggacgttgtagacacgtgtgatgcagctggcatcagtaaaaaggcggtcaaactgagaccaattgcggtcatcaagggataa

SEQ ID NO: 14 - protein, 505 aa, Caenorhabditis elegans
MPRTFEEECDFIDRLTDTKFRIKKGFVPNMNVEGRFYVNNSLEQLMFDELKFSCDGQGIGGFLPAVRQIANVASLPGIVGHSIGLPDIHSGYGFSIGNIAAFDVGNPESVISPGGVGFDINCGVRLLRTNLFEENVKPLKEQLTQSLFDHIPVGVGSRGAIPMLASDLVECLEMGMDWTLREGYSWAEDKEHCEEYGRMLQADASKVSLRAKKRGLPQLGTLGAGNHYAEVQVVDEIYDKHAASTMGIDEEGQVVVMLHCGSRGLGHQVATDSLVEMEKAMARDGIVVNDKQLACARINSVEGKNYFSGMAAAANFAWVNRSCITFCVRNAFQKTFGMSADDMDMQVIYDVSHNVAKMEEHMVDGRPRQLCVHRKGATRAFPAHHPLIPVDYQLIGQPVLIGGSMGTCSYVLTGTEQGLVETFGTTCHGAGRALSRAKSRRTITWDSVIDDLKKKEISIRIASPKLIMEEAPESYKNVTDVVDTCDAAGISKKAVKLRPIAVIKG

SEQ ID NO: 15 - DNA, 1518 bp, Homo sapiens
  CDS (1)..(1518)
atgagtcgcagctataatgatgagctgcagttcttggagaagatcaataaaaactgctggaggatcaagaagggcttcgtgcccaacatgcaggttgaaggtgttttctatgtgaatgatgctctggagaaattgatgtttgaggaattaaggaatgcctgtcgaggtggtggtgttggtggcttcctgccagccatgaaacagattggcaatgtggcagccctgcctggaattgttcatcgatctattgggcttcctgatgtccattcaggatatgggtttgctattgggaacatggcagcctttgatatgaatgaccctgaagcagtagtatccccaggtggtgtcgggtttgacatcaactgtggtgtccgcttgctaagaaccaatttagatgaaagtgatgtccagcctgtgaaggagcaacttgcccaagctatgtttgaccacattcctgttggggtggggtcaaaaggtgtcatcccaatgaatgccaaagacttggaggaggccttggagatgggggtggactggtccttaagagaagggtatgcctgggctgaagacaaggagcactgcgaggagtacggaaggatgctgcaggctgaccccaataaagtttctgcaagggcgaagaaaagaggccttcctcagttggggaccctgggagcaggcaaccattatgcagaaatccaggttgtggatgagattttcaatgagtatgctgctaaaaaaatgggcatcgaccataagggacaggtgtgtgtgatgatccacagtggaagcagaggcttgggccaccaagtagccacagatgcgctggtagctatggagaaggccatgaagagagacaagattatagtcaatgatcggcagttggcttgtgctcgaatcgcttccccagagggtcaagactatctgaagggaatggcagctgctgggaactatgcctgggtcaaccgctcttccatgaccttcttaacccgtcaggctttcgccaaggtcttcaacacaacccctgatgacttggacctacatgtgatttatgatgtttctcacaacattgccaaagtggagcagcatgtggtggacggaaaggaacggacactgttagtacacaggaagggatccacccgcgctttccctcctcaccatcccctcattgctgttgattaccaactcactggacagccagtgctcattggtggcaccatgggaacctgtagttatgttcttactggcactgaacagggcatgactgagacctttggaacaacctgtcatggagcgggccgtgcattgtcccgagcaaaatctcgacgtaatttagatttccaggatgtcttagacaaattggcagatatgggaattgcgatccgtgttgcctcacccaaactggttatggaagaggctcctgagtcctataagaatgtgacagatgtggtaaatacctgccatgatgctggaatcagcaagaaagccattaaactgagaccaattgctgtgatcaaaggatag

SEQ ID NO: 16 - protein, 505 aa, Homo sapiens
MSRSYNDELQFLEKINKNCWRIKKGFVPNMQVEGVFYVNDALEKLMFEELRNACRGGGVGGFLPAMKQIGNVAALPGIVHRSIGLPDVHSGYGFAIGNMAAFDMNDPEAVVSPGGVGFDINCGVRLLRTNLDESDVQPVKEQLAQAMFDHIPVGVGSKGVIPMNAKDLEEALEMGVDWSLREGYAWAEDKEHCEEYGRMLQADPNKVSARAKKRGLPQLGTLGAGNHYAEIQVVDEIFNEYAAKKMGIDHKGQVCVMIHSGSRGLGHQVATDALVAMEKAMKRDKIIVNDRQLACARIASPEGQDYLKGMAAAGNYAWVNRSSMTFLTRQAFAKVFNTTPDDLDLHVIYDVSHNIAKVEQHVVDGKERTLLVHRKGSTRAFPPHHPLIAVDYQLTGQPVLIGGTMGTCSYVLTGTEQGMTETFGTTCHGAGRALSRAKSRRNLDFQDVLDKLADMGIAIRVASPKLVMEEAPESYKNVTDVVNTCHDAGISKKAIKLRPIAVIKG

SEQ ID NO: 17 - DNA, 1695 bp, Caenorhabditis elegans
  CDS (1)..(1695)
atgggggttgtcaatctactatcccgatcatcatttccatctgtcgactcatatctggcattgtcagttcttgttgcaattgttgcttctgtcactgtatttacaacatttcgctcacaacccgagctacagaagcttatcgaagaagaacttcggaataatacacgactgtcatcagcatatggtttgaatattgaagcattgtccgggcacacattctttcaaattgctcattacattttatctgatacaacgctaatctgggttgctataaactcgtattttgccatactagcagtttgtacgagactaataatcaaattaacattcaaagagctcgcccgacaggaggaaaatgtggcccgtcaagctttcttctgttatgtcctgttaacaatcgtctacttatcagttgtgatcggaccgcaaaaggggcatcgtgtaatgccatggatgatctggggtggtatttgtgctttcttatcccatttgcagtttatcacatgtcagaggctcaagcatatctctccatcctgtgatcgtggaagtcagaagatttcattcctctcgttgttcctcttcttcgtttcgattgccatgacgttcttgatttctcgattccaacatcacttaacatggcaacctgctgtacttttatattttgattgccttctcgcagtattccgttccacttacattctgttccgatgcatttcttcatctcgtgtattctccttcaatcctgactctgttcgtcatttcaactattggctcgagcttatcaccaactttgtctgcgaactcattcaaatgctcagttttgctcaacttttggcattctcacctggacttaatctcacaagcatcttcttcttgtatcacatgaagcttacctacaactgcatgactgaacaattaagtcgtcatcgtaatcataagaagatcttcgaacacattgagagatcatatccaagtgttaaatgtgctaatggtgatgatcgttgcgttgtttgctgggaattgctcggaacatctcgaagacttccatgctctcatcaattccatgattggtgtctcatgtggtggcttgctcaggattcttcatgtccaacgtgtcgttgtactattccatctccacaagatcaaatcagacaaccaccagaggttggaaatagtacaagattacgtttcaatggtggatcatttggattcgttcatttcccagcattcactcttgaagtagctgctaacttcgggcctttctttggtcgagcagccgaaccaacagaagagcaattgcaaacaatgctcgagcaagtcagggagatgttcccgcagatgtctgttgatataataatgacggatctacgacaatcgggatccgctcagtctacaatcgaaaatattctagaaggaaggataggaatgaatgcatcatttatgcccggtggggttttggatgatgaactctcagatgagagtgaaaacgaaatagaatatgaagagcccgccgaaatagtccaagaaccagataatggccgtcaaagaacgtggacaaaattgagttcttcatcaggagatgaagatctatcatactatgaaatccagcgagccaaaatgatcgaaacttaccgacggaaatatctggagtctgataaagctgccgatctacgagcaatgggaatcaccgaataa

SEQ ID NO: 18 - protein, 564 aa, Caenorhabditis elegans
MGVVNLLSRSSFPSVDSYLALSVLVAIVASVTVFTTFRSQPELQKLIEEELRNNTRLSSAYGLNIEALSGHTFFQIAHYILSDTTLIWVAINSYFAILAVCTRLIIKLTFKELARQEENVARQAFFCYVLLTIVYLSVVIGPQKGHRVMPWMIWGGICAFLSHLQFITCQRLKHISPSCDRGSQKISFLSLFLFFVSIAMTFLISRFQHHLTWQPAVLLYFDCLLAVFRSTYILFRCISSSRVFSFNPDSVRHFNYWLELITNFVCELIQMLSFAQLLAFSPGLNLTSIFFLYHMKLTYNCMTEQLSRHRNHKKIFEHIERSYPSVKCANGDDRCVVCWELLGTSRRLPCSHQFHDWCLMWWLAQDSSCPTCRCTIPSPQDQIRQPPEVGNSTRLRFNGGSFGFVHFPAFTLEVAANFGPFFGRAAEPTEEQLQTMLEQVREMFPQMSVDIIMTDLRQSGSAQSTIENILEGRIGMNASFMPGGVLDDELSDESENEIEYEEPAEIVQEPDNGRQRTWTKLSSSSGDEDLSYYEIQRAKMIETYRRKYLESDKAADLRAMGITE

SEQ ID NO: 19 - DNA, 1932 bp, Homo sapiens
  CDS (1)..(1932)
atgccgctgctcttcctcgagcgcttcccctggcccagcctccgcacctacacgggcctcagcggcctggccctgctgggcaccatcatcagcgcctaccgcgcgctcagccagcccgaggccggccccggcgagccggaccagctaacggcctcgctgcagcctgagccgccggcgcccgcccggccgagcgccgggggaccccgggcccgcgatgtggcccagtacctgctctcagacagcctcttcgtgtgggttctagtaaataccgcttgctgtgttttgatgttggtggctaagctcatccagtgtattgtgtttggccctcttcgagtgagtgagagacagcatctcaaagacaaattttggaattttattttctacaagttcattttcatctttggtgtgctgaatgtccagacagtggaagaggtggtcatgtggtgcctctggtttgccggacttgtctttctgcacctgatggttcagctctgcaaggatcgatttgaatatctttccttctcgcccaccacgccgatgagcagccacggtcgagtcctgtccctgttggttgccatgctgctttcctgctgtggactggcggccgtctgctccatcaccggctacacccacggaatgcacaccttggctttcatggctgcagagtctcttcttgtgacagtgaggactgctcatgtgattttacgatacgtaattcacctctgggacctcaaccacgaagggacgtgggaaggaaaggggacgtatgtctattacacagactttgtcatggagctcactctcctgtccctggacctcatgcaccatattcacatgttgttatttggcaacatctggttatccatggccagcctggtcatctttatgcagctgcgttacctgtttcatgaggtgcaacgtcgaattcgtcggcacaagaactatctacgtgtggttggaaacatggaggccaggtttgcagttgcaactccagaggagctggctgtcaacaatgacgactgtgccatctgttgggactccatgcaggctgcgcggaaactgccctgtggacatcttttccacaactcctgtcttcgttcctggctagaacaagacacctcctgtccaacatgcagaatgtctcttaatattgccgacaataatcgtgtcagggaagaacatcaaggagagaacttggatgagaatttggttcctgtagcagcagccgaagggagacctcgcttaaaccaacacaatcacttcttccatttcgatgggtctcggattgcgagctggctgccgagtttttcggttgaagtgatgcacaccaccaacattcttggcattacgcaggccagcaactcccagctcaatgcaatggctcatcagattcaagagatgtttccccaggttccataccatctggtactgcaggacctccagctgacacgctcagttgaaataacaacagacaatattttagaaggacggattcaagtaccttttcctacacagcggtcagatagcatcagacctgcattgaacagtcctgtggaaaggccaagcagtgaccaggaagagggagaaacttctgctcagaccgagcgtgtgccactggacctcagtcctcgcctggaggagacgctggacttcggcgaggtggaagtggagcccagtgaggtggaagacttcgaggctcgtgggagccgcttctccaagtctgctgatgagagacagcgcatgctggtgcagcgtaaggacgaactcctccagcaagctcgcaaacgtttcttgaacaaaagttctgaagatgatgcggcctcagagagcttcctcccctcggaaggtgcgtcctctgaccccgtgaccctgcgtcgaaggatgctggctgccgccgcggaacggaggcttcagaagcagcagacctcctag

SEQ ID NO: 20 - protein, 643 aa, Homo sapiens
MPLLFLERFPWPSLRTYTGLSGLALLGTIISAYRALSQPEAGPGEPDQLTASLQPEPPAPARPSAGGPRARDVAQYLLSDSLFVWVLVNTACCVLMLVAKLIQCIVFGPLRVSERQHLKDKFWNFIFYKFIFIFGVLNVQTVEEVVMWCLWFAGLVFLHLMVQLCKDRFEYLSFSPTTPMSSHGRVLSLLVAMLLSCCGLAAVCSITGYTHGMHTLAFMAAESLLVTVRTAHVILRYVIHLWDLNHEGTWEGKGTYVYYTDFVMELTLLSLDLMHHIHMLLFGNIWLSMASLVIFMQLRYLFHEVQRRIRRHKNYLRVVGNMEARFAVATPEELAVNNDDCAICWDSMQAARKLPCGHLFHNSCLRSWLEQDTSCPTCRMSLNIADNNRVREEHQGENLDENLVPVAAAEGRPRLNQHNHFFHFDGSRIASWLPSFSVEVMHTTNILGITQASNSQLNAMAHQIQEMFPQVPYHLVLQDLQLTRSVEITTDNILEGRIQVPFPTQRSDSIRPALNSPVERPSSDQEEGETSAQTERVPLDLSPRLEETLDFGEVEVEPSEVEDFEARGSRFSKSADERQRMLVQRKDELLQQARKRFLNKSSEDDAASESFLPSEGASSDPVTLRRRMLAAAAERRLQKQQTS

SEQ ID NO: 21 - DNA, 564 bp, Caenorhabditis elegans
  CDS (1)..(564)
atggctcaaaaatcggctttaatcatattggcggccgaaggagctgaggaaatggaggtcattatcactggagatgtacttgctcgtggtgaaattcgtgtggtttatgccggattagatggagccgaaccggtaaaatgtgctcgcggagcccacatcgtgccagacgtcaaactcgaagacgtggaaaccgaaaaattcgatattgtgattcttccaggcggccaaccgggcagcaacacgttggctgaaagcctacttgtccgcgatgttctcaagagccaagtagagtctggtgggctgattggagcaatttgtgcagctccaattgcactcttgagccatggagtcaaggcagaacttgtgacaagtcatccaagtgttaaggagaaactcgagaaaggaggctacaagtactcggaggatcgtgttgttgtcagtggcaaaatcatcacctctcgtggacccggaactgccttcgaatttgcgctgaaaattgtggagctgcttgagggaaaggacaaggccaccagccttattgctccgatgctcctgaagctctaa

SEQ ID NO: 22 - protein, 187 aa, Caenorhabditis elegans
MAQKSALIILAAEGAEEMEVIITGDVLARGEIRVVYAGLDGAEPVKCARGAHIVPDVKLEDVETEKFDIVILPGGQPGSNTLAESLLVRDVLKSQVESGGLIGAICAAPIALLSHGVKAELVTSHPSVKEKLEKGGYKYSEDRVVVSGKIITSRGPGTAFEFALKIVELLEGKDKATSLIAPMLLKL

SEQ ID NO: 23 - DNA, 570 bp, Homo sapiens
  CDS (1)..(570)
atggcttccaaaagagctctggtcatcctggctaaaggagcagaggaaatggagacggtcatccctgtagatgtcatgaggcgagctgggattaaggtcaccgttgcaggcctggctggaaaagacccagtacagtgtagccgtgatgtggtcatttgtcctgatgccagccttgaagatgcaaaaaaagagggaccatatgatgtggtggttctaccaggaggtaatctgggcgcacagaatttatctgagtctgctgctgtgaaggagatactgaaggagcaggaaaaccggaagggcctgatagccgccatctgtgcaggtcctactgctctgttggctcatgaaataggttttggaagtaaagttacaacacaccctcttgctaaagacaaaatgatgaatggaggtcattacacctactctgagaatcgtgtggaaaaagacggcctgattcttacaagccgggggcctgggaccagcttcgagtttgcgcttgcaattgttgaagccctgaatggcaaggaggtggcggctcaagtgaaggctccacttgttcttaaagactag

SEQ ID NO: 24 - protein, 189 aa, Homo sapiens
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 25 - DNA, 1926 bp, Caenorhabditis elegans
  CDS (1)..(1926)
atgtctatgaaacgattcggaaaagcagcatatcgaatcgcaaatgagttagttgcaaaaggtggacgactaccaattttccaacgcttcctgccgagaatatttcccgccacttataatttaggagttcatgtcgtactcaaaaaggctccatttccacgacaaaatgctctacgaattgctcgccttgtaactcgccacggtcgagttttccggccattttcctcagtaataatcgaaagacatcgatttcaaaatcaaaatgattggcgtcgaaagtttcaaccgattcgtaaagaattgccaagaaatgtggatttagtcgaacgaatcaggcagatatttggcaattctctacgatacaatgaggatttgaaaagcactgaatggccgaatagaattgattcttatgagtttggggaatttctcggtcaaggatgcaatgcagcagtttactctgcgagattagccaattctgatgcagaatcctcagggaatactcactatggtgcagggtttaatgaagtcacaaatatacttgcagaaattccgccagttagcaaagttgcacaaaagaaattcccgttggcaatcaaattaatgtttaattttgaacatgatcgcgatggagatgctcatctcttgaaatcaatgggaaatgaattggctccatatccgaatgctgcaaagttgctcaatggacaaatgggaacatttagacctcttccagcaaaacatccaaatgttgttcgaattcagacagcttttattgattcgttaaaagttttgccagatgcgattgaacgatatccagatgcccttcacactgcacgttggtatgagtcaattgcctccgaaccgaaaacaatgtacgtagtaatgagacgataccgacaaacacttcatgaatatgtatggactcgtcatcgaaattattggacaggacgagtgataattgctcaactattagaagcatgtacatatcttcataagcataaagttgctcagcgagacatgaaaagtgataatattcttctggaatatgattttgacgacgagattccccaattagttgtcgccgattttggatgtgcacttgcatgtgacaattggcaagtagactatgaatcagatgaagttagtcttggaggaaatgccaagacaaaagcaccagaaattgcgacggctgttcctggaaagaatgtaaaagtaaacttcgaaatggcagatacatgggcagctggaggcctttcttatgaagttctaacacgatcaaatccattctacaaacttcttgatactgcaacataccaggaatcagaactaccagcactcccatctcgtgtcaattttgtggcacgagatgtcatttttgacctactcaagcgagatcctaatgaaagagtcaagccgaatattgctgcaaatgcgttgaatttgtcattgttcagaatgggagaagatgtgaagcagatgatggaaaaatgtggaatatctcaaatgactactctattggctggaagttctaaagttttgagtcaaaaaatcaatagtcgtctggacaaagtgatgaatctgattactgctgaaactatcatggccaacctagctccacatttgattagtcgagcagaacgacaacttcgagcaacatttctttcaagaatgaatcgagaagatatttggagaagtcttcaatatttcttcccagctggtgttcaacttgacacacctgccacatcatcagactgtttggagactatttccagtttgatgtcgagtttttcaaatgattcagaaaattacgagaagcaacagaaaccggctaaaaatggatacaacaatgttccacttcttctcagaaatgttatccgtacagatgcggatggaatcaatggaattgtacatagagttcgatctaaatag

SEQ ID NO: 26 - protein, 641 aa, Caenorhabditis elegans
MSMKRFGKAAYRIANELVAKGGRLPIFQRFLPRIFPATYNLGVHVVLKKAPFPRQNALRIARLVTRHGRVFRPFSSVIIERHRFQNQNDWRRKFQPIRKELPRNVDLVERIRQIFGNSLRYNEDLKSTEWPNRIDSYEFGEFLGQGCNAAVYSARLANSDAESSGNTHYGAGFNEVTNILAEIPPVSKVAQKKFPLAIKLMFNFEHDRDGDAHLLKSMGNELAPYPNAAKLLNGQMGTFRPLPAKHPNVVRIQTAFIDSLKVLPDAIERYPDALHTARWYESIASEPKTMYVVMRRYRQTLHEYVWTRHRNYWTGRVIIAQLLEACTYLHKHKVAQRDMKSDNILLEYDFDDEIPQLVVADFGCALACDNWQVDYESDEVSLGGNAKTKAPEIATAVPGKNVKVNFEMADTWAAGGLSYEVLTRSNPFYKLLDTATYQESELPALPSRVNFVARDVIFDLLKRDPNERVKPNIAANALNLSLFRMGEDVKQMMEKCGISQMTTLLAGSSKVLSQKINSRLDKVMNLITAETIMANLAPHLISRAERQLRATFLSRMNREDIWRSLQYFFPAGVQLDTPATSSDCLETISSLMSSFSNDSENYEKQQKPAKNGYNNVPLLLRNVIRTDADGINGIVHRVRSK

SEQ ID NO: 27 - DNA, 1746 bp, Homo sapiens
  CDS (1)..(1746)
atggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcatcgaggaaaaacaggcggagagccggcgggcggtctcggcctgtcaggagatccaggcaatttttacccagaaaagcaagccggggcctgacccgttggacacgagacgcttgcagggctttcggctggaggagtatctgatagggcagtccattggtaagggctgcagtgctgctgtgtatgaagccaccatgcctacattgccccagaacctggaggtgacaaagagcaccgggttgcttccagggagaggcccaggtaccagtgcaccaggagaagggcaggagcgagctccgggggcccctgccttccccttggccatcaagatgatgtggaacatctcggcaggttcctccagcgaagccatcttgaacacaatgagccaggagctggtcccagcgagccgagtggccttggctggggagtatggagcagtcacttacagaaaatccaagagaggtcccaagcaactagcccctcaccccaacatcatccgggttctccgcgccttcacctcttccgtgccgctgctgccaggggccctggtcgactaccctgatgtgctgccctcacgcctccaccctgaaggcctgggccatggccggacgctgttcctcgttatgaagaactatccctgtaccctgcgccagtacctttgtgtgaacacacccagcccccgcctcgccgccatgatgctgctgcagctgctggaaggcgtggaccatctggttcaacagggcatcgcgcacagagacctgaaatccgacaacatccttgtggagctggacccagacggctgcccctggctggtgatcgcagattttggctgctgcctggctgatgagagcatcggcctgcagttgcccttcagcagctggtacgtggatcggggcggaaacggctgtctgatggccccagaggtgtccacggcccgtcctggccccagggcagtgattgactacagcaaggctgatgcctgggcagtgggagccatcgcctatgaaatcttcgggcttgtcaatcccttctacggccagggcaaggcccaccttgaaagccgcagctaccaagaggctcagctacctgcactgcccgagtcagtgcctccagacgtgagacagttggtgagggcactgctccagcgagaggccagcaagagaccatctgcccgagtagccgcaaatgtgcttcatctaagcctctggggtgaacatattctagccctgaagaatctgaagttagacaagatggttggctggctcctccaacaatcggccgccactttgttggccaacaggctcacagagaagtgttgtgtggaaacaaaaatgaagatgctctttctggctaacctggagtgtgaaacgctctgccaggcagccctcctcctctgctcatggagggcagccctgtga

SEQ ID NO: 28 - protein, 581 aa, Homo sapiens
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLIEEKQAESRRAVSACQEIQAIFTQKSKPGPDPLDTRRLQGFRLEEYLIGQSIGKGCSAAVYEATMPTLPQNLEVTKSTGLLPGRGPGTSAPGEGQERAPGAPAFPLAIKMMWNISAGSSSEAILNTMSQELVPASRVALAGEYGAVTYRKSKRGPKQLAPHPNIIRVLRAFTSSVPLLPGALVDYPDVLPSRLHPEGLGHGRTLFLVMKNYPCTLRQYLCVNTPSPRLAAMMLLQLLEGVDHLVQQGIAHRDLKSDNILVELDPDGCPWLVIADFGCCLADESIGLQLPFSSWYVDRGGNGCLMAPEVSTARPGPRAVIDYSKADAWAVGAIAYEIFGLVNPFYGQGKAHLESRSYQEAQLPALPESVPPDVRQLVRALLQREASKRPSARVAANVLHLSLWGEHILALKNLKLDKMVGWLLQQSAATLLANRLTEKCCVETKMKMLFLANLECETLCQAALLLCSWRAAL

SEQ ID NO: 29 - DNA, 1074 bp, Caenorhabditis elegans
  CDS (1)..(1074)
atgcaaggacaaggatcaccattctgccgttctagaacccgctcgagatctcgtggagctttcaatcgtagctccaaccacggttcagtgcttccaattcagcctgccgtattggattcaattgccgaggaatcatcgtctactatcatgacagtagtcaatcctttgatggtggcagcaagacagctcaaatttgcttgccagatggctcatggaagtccagttggaatcatcgacaaatggaacaatatggaggaattgtatcaatcaattgccgattgctttacaatttcaaaagatgatatcattttcctgacagtcaatgattttaagccagacatgaagaacatgttcactggaacactgaacttcaaggatatgctcttcgcccacattcgcggacaagcaaccgagctccgagtcgtcaaagatgctaaaaactttggagtgacaatcacggacaacggactcgggaatgcatttatcaaggtcataagcccagattcggtgtttgatcgtatgcgtccagccactcaagttggtcaactcattgaggcggtcaatggagaatgtgtgctcgggaaaaggcattaccaagttgcccgaattttgaaaaatgttcgtcgcggcgaggaatgcgtggttcgattgattgctccaaagactgctgatccaggaaccatgaagacgactggaaaaactggcggaggtctggcaaaaggaaccattcgtttcaaatcggaaggcggatttgctgttgaggatgttcaagatcagatgatccaagctgagatgtgcggaaagctcaatgagatattcgaccaatacttgggagttcaagatgatcagctggccatgagaatctgggaaactgcttcaaactgcgagacgcttttgcaactcagtgaagccatcaaagagtcggagctctcaatgttcgatttcccggatggaatggttttcgatatgtggggaatcatcggtgatttgaagcgggagcaacgtgagaagaagccatccccagtcatgaagaatgcgatttcaagaccatcggctatgaaactctttgagtaa

SEQ ID NO: 30 - protein, 357 aa, Caenorhabditis elegans
MQGQGSPFCRSRTRSRSRGAFNRSSNHGSVLPIQPAVLDSIAEESSSTIMTVVNPLMVAARQLKFACQMAHGSPVGIIDKWNNMEELYQSIADCFTISKDDIIFLTVNDFKPDMKNMFTGTLNFKDMLFAHIRGQATELRVVKDAKNFGVTITDNGLGNAFIKVISPDSVFDRMRPATQVGQLIEAVNGECVLGKRHYQVARILKNVRRGEECVVRLIAPKTADPGTMKTTGKTGGGLAKGTIRFKSEGGFAVEDVQDQMIQAEMCGKLNEIFDQYLGVQDDQLAMRIWETASNCETLLQLSEAIKESELSMFDFPDGMVFDMWGIIGDLKREQREKKPSPVMKNAISRPSAMKLFE

SEQ ID NO: 31 - DNA, 1002 bp, Homo sapiens
  CDS (1)..(1002)
atgccgctgggactggggcggcggaaaaaggcgccccctctagtggaaaatgaggaggctgagccaggccgtggagggctgggcgtgggggagccagggcctctgggcggaggtgggtcggggggcccccaaatgggcttgcccccccctcccccagccctgcggccccgcctcgtgttccacacccagctggcccatggcagtcccactggccgcatcgagggcttcaccaacgtcaaggagctgtatggcaagatcgccgaggccttccgcctgccaactgccgaggtgatgttctgcaccctgaacacccacaaagtggacatggacaagctcctggggggccagatcgggctggaggacttcatcttcgcccacgtgaaggggcagcgcaaggaggtggaggtgttcaagtcggaggatgcactcgggctcaccatcacggacaacggggctggctacgccttcatcaagcgcatcaaggagggcagcgtgatcgaccacatccacctcatcagcgtgggcgacatgatcgaggccattaacgggcagagcctgctgggctgccggcactacgaggtggcccggctgctcaaggagctgccccgaggccgtaccttcacgctgaagctcacggagcctcgcaaggccttcgacatgatcagccagcgttcagcgggtggccgccctggctctggcccacaactgggcactggccgagggaccctgcggctccgatcccggggccccgccacggtggaggatctgccctctgcctttgaagagaaggccattgagaaggtggatgacctgctggagagttacatgggtatcagggacacggagctggcggccaccatggtggagctgggaaaggacaaaaggaacccggatgagctggccgaggccctggacgaacggctgggtgactttgccttccctgacgagttcgtctttgacgtctggggcgccattggggacgccaaggtcggccgctactag

SEQ ID NO: 32 - protein, 333 aa, Homo sapiens
MPLGLGRRKKAPPLVENEEAEPGRGGLGVGEPGPLGGGGSGGPQMGLPPPPPALRPRLVFHTQLAHGSPTGRIEGFTNVKELYGKIAEAFRLPTAEVMFCTLNTHKVDMDKLLGGQIGLEDFIFAHVKGQRKEVEVFKSEDALGLTITDNGAGYAFIKRIKEGSVIDHIHLISVGDMIEAINGQSLLGCRHYEVARLLKELPRGRTFTLKLTEPRKAFDMISQRSAGGRPGSGPQLGTGRGTLRLRSRGPATVEDLPSAFEEKAIEKVDDLLESYMGIRDTELAATMVELGKDKRNPDELAEALDERLGDFAFPDEFVFDVWGAIGDAKVGRY

SEQ ID NO: 33 - DNA, 1293 bp, Caenorhabditis elegans
  CDS (1)..(1293)
atggtcagcgtggatccattggcaacggagcgttggaggagcatcagaagattaacagatcgcgactctgcatataaagttccatggtttgttcccggaccggaaaactttgaagcactgcaaaacacgaagattcttgttattggtgctggtggattgggttgtgagttgttaaagaacttggctttgagcggattccgaacaattgaagttatcgatatggacacaattgatgtatcaaacctaaatcgtcaattcttatttagggaatctgacgttggaaaatcaaaagctgaggtagctgctgctttcgttcaacaaagagttgttggatgtcaggtgactgcgcacaactgtagaattgaagacaagggtcaagaattttatcgaaaattctctataattatctgtggacttgattcgattccagccagaagatggatcaacggaatgctgtgtgatttggttttggaaatggccgacggaaaacctgatgagaacacaattattccaatgattgacggaggaactgaaggatttaaaggaaatgctcgtgtcatatatccgaaattcacagcctgtattgattgtactcttgatctttatccacctcaagtcaattttccattgtgcacaattgctcacactcctcgacttccagaacattgtattgaatacattaaagtagttgtttggccagaggaaaaaccatttgaaggtgtttctcttgacgctgacgatccgatccacgttgaatgggttctcgaaagagcaagtcttcgtgcagaaaaatacaatattcgaggtgtggatcgtcgtttgacatctggagttctaaaaagaattattccggctgttgcatcaacaaatgcagttatcgctgcatcatgtgccctagaagctctgaaattggccacaaacattgccaaaccaatcgataattatcttaatttcactcaaattcacggagcatataccagtgttgtttcaatgatgaaagatgacaattgtctcacttgtagtggtgggcgtcttccattcgaagtttcaccatcttcaactcttgaatcgcttatcatcagactctcggagcgtttccatctcaaacatccgacactagcgacctcaactcgaaaactttactgtattagcagtttcatgccacaatttgaacaagaaagcaaagaaaatctgcatacttcgatgaaagatcttgttagcgatggcgaagaaatactggtatccgacgaagcattgtctcgtgcacttacactacgaattcagctcatctaa

SEQ ID NO: 34 - protein, 430 aa, Caenorhabditis elegans
MVSVDPLATERWRSIRRLTDRDSAYKVPWFVPGPENFEALQNTKILVIGAGGLGCELLKNLALSGFRTIEVIDMDTIDVSNLNRQFLFRESDVGKSKAEVAAAFVQQRVVGCQVTAHNCRIEDKGQEFYRKFSIIICGLDSIPARRWINGMLCDLVLEMADGKPDENTIIPMIDGGTEGFKGNARVIYPKFTACIDCTLDLYPPQVNFPLCTIAHTPRLPEHCIEYIKVVVWPEEKPFEGVSLDADDPIHVEWVLERASLRAEKYNIRGVDRRLTSGVLKRIIPAVASTNAVIAASCALEALKLATNIAKPIDNYLNFTQIHGAYTSVVSMMKDDNCLTCSGGRLPFEVSPSSTLESLIIRLSERFHLKHPTLATSTRKLYCISSFMPQFEQESKENLHTSMKDLVSDGEEILVSDEALSRALTLRIQLI

SEQ ID NO: 35 - DNA, 1392 bp, Homo sapiens
  CDS (1)..(1392)
atggcggatggcgaggagccggagaagaaaagaaggagaatagaggagctgctggctgagaaaatggctgttgatggtgggtgtggggacactggagactgggaaggtcgctggaaccatgtaaagaagttcctcgagcgatctggacccttcacacaccctgatttcgaaccgagcactgaatctctccagttcttgttagatacatgtaaagttctagtcattggagctggcggcttaggatgtgagctcctgaaaaatctggccttgtctggttttagacagattcatgttatagatatggacactatagatgtttccaatctaaataggcagtttttatttaggcctaaagatattggaagacctaaggctgaagttgctgcagaatttctaaatgacagagttcctaattgcaatgtagttccacatttcaacaagattcaagattttaacgacactttctatcgacaatttcatattattgtatgtggactggactctatcatcgccagaagatggataaatggcatgctgatatctcttctaaattatgaagatggtgtcttagatccaagctccattgtccctttgatagatggggggacagaaggttttaaaggaaatgcccgggtgattctgcctggaatgactgcttgtatcgaatgcacgctggaactttatccaccacaggttaattttcccatgtgcaccattgcatctatgcccaggctaccagaacactgtattgagtatgtaaggatgttgcagtggcctaaggagcagccttttggagaaggggttccattagatggagatgatcctgaacatatacaatggattttccaaaaatccctagagagagcatcacaatataatattaggggtgttacgtataggctcactcaaggggtagtaaaaagaatcattcctgcagtagcttccacaaatgcagtcattgcagctgtgtgtgccactgaggtttttaaaatagccacaagtgcatacattcccttgaataattacttggtgtttaatgatgtagatgggctgtatacatacacatttgaagcagaaagaaaggaaaactgcccagcttgtagccagcttcctcaaaatattcagttttctccatcagctaaactacaggaggttttggattatctaaccaatagtgcttctctgcaaatgaaatctccagccatcacagccaccctagagggaaaaaatagaacactttacttacagtcggtaacctctattgaagaacgaacaaggccaaatctctccaaaacattgaaagaattggggcttgttgatggacaagaactggcggttgctgatgtcaccaccccacagactgtactattcaaacttcattttacttcttaa

SEQ ID NO: 36 - protein, 463 aa, Homo sapiens
MADGEEPEKKRRRIEELLAEKMAVDGGCGDTGDWEGRWNHVKKFLERSGPFTHPDFEPSTESLQFLLDTCKVLVIGAGGLGCELLKNLALSGFRQIHVIDMDTIDVSNLNRQFLFRPKDIGRPKAEVAAEFLNDRVPNCNVVPHFNKIQDFNDTFYRQFHIIVCGLDSIIARRWINGMLISLLNYEDGVLDPSSIVPLIDGGTEGFKGNARVILPGMTACIECTLELYPPQVNFPMCTIASMPRLPEHCIEYVRMLQWPKEQPFGEGVPLDGDDPEHIQWIFQKSLERASQYNIRGVTYRLTQGVVKRIIPAVASTNAVIAAVCATEVFKIATSAYIPLNNYLVFNDVDGLYTYTFEAERKENCPACSQLPQNIQFSPSAKLQEVLDYLTNSASLQMKSPAITATLEGKNRTLYLQSVTSIEERTRPNLSKTLKELGLVDGQELAVADVTTPQTVLFKLHFTS

SEQ ID NO: 37 - DNA, 486 bp, Caenorhabditis elegans
  CDS (1)..(486)
atgggtgatgacgaccgttgcaagacgggaattcttatccggtgtcttggcgatcttcaagaaatggaggacggctaccttcgcaaaatggaagtcatggagaaagaacaggtggctgccgaaaaacgacttgttgaatgcagggaagacgttgcaaagttacgggcagaaaacgcacagttggcgaccgatatcgacaatctcaagactgcaaccgaaaacaccggacgcctcaatgcagaaattgcccagttacgaacggaactgtccgccgtgccccgcccttgttgcgcggtttgtcacgattcatacgcctcccgcggtccaaaaaaacccaaggtttgttcctgtctgcacacttattgtggcgcgtgcattagggaaatttcgagccgccacaatggtgaaatgaaatgccccgaatgcgttgcggacgtccgcatactgggaacaaactttgggataacaaatgcgtttcggtcatga

SEQ ID NO: 38 - protein, 161 aa, Caenorhabditis elegans
MGDDDRCKTGILIRCLGDLQEMEDGYLRKMEVMEKEQVAAEKRLVECREDVAKLRAENAQLATDIDNLKTATENTGRLNAEIAQLRTELSAVPRPCCAVCHDSYASRGPKKPKVCSCLHTYCGACIREISSRHNGEMKCPECVADVRILGTNFGITNAFRS

SEQ ID NO: 39 - DNA, 2235 bp, Homo sapiens
  CDS (1)..(2235)
atggccagtgaaggcaccaacatcccaagtcctgtggtgcgccagattgacaagcagtttctgatttgcagtatatgcctggaacggtacaagaatcccaaggttctcccctgtctgcacactttctgcgagaggtgcctgcagaactacattcctgcccacagtttaaccctctcctgcccagtgtgccgccagacctccatcctgcccgagaaaggggtggccgcgctccagaacaatttcttcatcacaaacctgatggacgtgctgcagcgaactccaggcagcaacgctgaggagtcttccatcctggagacagtcactgctgtggctgcgggaaagcctctctcttgcccaaaccacgatgggaatgtgatggaattttactgccagtcctgtgagactgccatgtgtcgggagtgcacggagggggagcacgcagagcaccccacagttccactcaaggatgtggtggaacagcacaaggcctcgctccaggtccagctggatgctgtcaacaaaaggctcccagaaatagattctgctcttcagttcatctctgaaatcattcatcagttaaccaaccaaaaggccagcatcgtggatgacattcattccacctttgatgagctccagaagactttaaatgtgcgcaagagtgtgctgcttatggaattggaggtcaactatggcctcaaacacaaagtcctccagtcgcagctggatactctgctccaggggcaggagagcattaagagctgcagcaacttcacagcgcaggccctcaaccatggcacggagaccgaggtcctactggtgaagaagcagatgagcgagaagctgaacgagctggccgaccaggacttccccttgcacccgcgggagaacgaccagctggatttcatcgtggaaaccgaggggctgaagaagtccatccacaacctcgggacgatcttaaccaccaacgccgttgcctcagagacagtggccacgggcgaggggctgcggcagaccatcatcgggcagcccatgtccgtcaccatcaccaccaaggacaaagacggtgagctgtgcaaaaccggcaacgcctacctcaccgccgaactgagcacccccgacgggagcgtggcagacggggagatcctggacaacaagaacggcacctatgagtttttgtacactgtccagaaggaaggggactttaccctgtctctgagactctatgaccagcacatccgaggcagcccgtttaagctgaaagtgatccgatccgctgatgtgtctcccaccacagaaggcgtgaagaggcgcgttaagtccccggggagcggccacgtcaagcagaaagctgtgaaaagacccgcaagcatgtacagcactggaaaacgaaaagagaatcccatcgaagacgatttgatctttcgagtgggtaccaaaggaagaaataaaggagagtttacaaatcttcagggggtagctgcatctacaaatggaaagatattaattgcagacagtaacaaccaatgtgtgcagatattttccaatgatggccagttcaaaagtcgttttggcatacggggacgctctccggggcagctgcagcggcccacaggagtggctgtacatcccagtggggacataatcattgccgattatgataataaatgggtcagcattttctcctccgatgggaaatttaagacaaaaattggatcaggaaagctgatgggacccaaaggagtttctgtggaccgcaatgggcacattattgttgtggacaacaaggcgtgctgcgtgtttatcttccagccaaacgggaaaatagtcaccaggtttggtagccgaggaaatggggacaggcagtttgcaggtccccattttgcagctgtaaatagcaataatgagattattattacagatttccataatcattctgtcaaggtgtttaatcaggaaggagaattcatgttgaagtttggctcaaatggagaaggaaatgggcagtttaatgctccaacaggtgtagcagtggattcaaatggaaacatcattgtggccgactggggaaacagcaggatccaggtttttgatgggagtggatcatttttgtcctacattaacacatctgctgacccactctatggcccccaaggcctggccctaacttcagatggtcatgttgtggttgcagactctggaaatcactgtttcaaagtctatcgatacttacagtaa

SEQ ID NO: 40 - protein, 744 aa, Homo sapiens
MASEGTNIPSPVVRQIDKQFLICSICLERYKNPKVLPCLHTFCERCLQNYIPAHSLTLSCPVCRQTSILPEKGVAALQNNFFITNLMDVLQRTPGSNAEESSILETVTAVAAGKPLSCPNHDGNVMEFYCQSCETAMCRECTEGEHAEHPTVPLKDVVEQHKASLQVQLDAVNKRLPEIDSALQFISEIIHQLTNQKASIVDDIHSTFDELQKTLNVRKSVLLMELEVNYGLKHKVLQSQLDTLLQGQESIKSCSNFTAQALNHGTETEVLLVKKQMSEKLNELADQDFPLHPRENDQLDFIVETEGLKKSIHNLGTILTTNAVASETVATGEGLRQTIIGQPMSVTITTKDKDGELCKTGNAYLTAELSTPDGSVADGEILDNKNGTYEFLYTVQKEGDFTLSLRLYDQHIRGSPFKLKVIRSADVSPTTEGVKRRVKSPGSGHVKQKAVKRPASMYSTGKRKENPIEDDLIFRVGTKGRNKGEFTNLQGVAASTNGKILIADSNNQCVQIFSNDGQFKSRFGIRGRSPGQLQRPTGVAVHPSGDIIIADYDNKWVSIFSSDGKFKTKIGSGKLMGPKGVSVDRNGHIIVVDNKACCVFIFQPNGKIVTRFGSRGNGDRQFAGPHFAAVNSNNEIIITDFHNHSVKVFNQEGEFMLKFGSNGEGNGQFNAPTGVAVDSNGNIIVADWGNSRIQVFDGSGSFLSYINTSADPLYGPQGLALTSDGHVVVADSGNHCFKVYRYLQ

SEQ ID NO: 41 - DNA, 1944 bp, Caenorhabditis elegans
  CDS (1)..(1944)
atggccacgtttgttccctttgttacttgtcttgatacaggtttttggaacgaagtgaacaagaaaaagctcaatgattggaaactcgatgagacgccaaagtgtatctcaagtcaattatcgcttcatcaaaccgaaggttttaaatgtcatctatccttaagttacgacagtttgagcagtttggagagcacaactggattgtcaatgtccggtacccttcttctgtacaatacaattgaaagtttcaagatggttgacaaaagtgatttgataagaagtgaagcagaaaagatctgggaatctatcacaactcgaaaatggctccaaaatcctcgtcttctgtcgcaattcttcatcatcgcttttgctgatttgaaaaaattcaaatattattattggacatgtgttcctgctttggtatatcctagtgaaataaaacaagagatcacaccactatcatctctcggagccgatcacaagatcctgttcgacttttataggaaaaacaattttccgatcttcctatactccaaacaatcttcaaaaatgctcgaactgtcagaattggaaaacaacacaaatcctgacgaaatatgtgttgttgtcgctgatccttctcctgttgcgtattctgcaggatggatggttcgcaacgttcttgccgcagtggcacatcttcatcctacgtggaagcattgtcatatcatcagtcttcgttcagctgacagtatcggaataaaatacacatggaccttgccgagtgctgaatgctcagcagatggagctcagaatgctgttccaaaagctgtgggatgggaaagaaatgcaaatgataagttgcagccgatttcagtagacttaagtaaagagttcgatccaaaaatattaatggaaagatctgtggatcttaatttatcattaatcaaatggagattgcacccagatattcaattggaaagatactcacagctgaaggttctcattcttggtgctggaacgcttggatgtaacattgcccgttgtcttatcggatggggagttcgtcacatttcgtttcttgataattcaactgtcagctacaataaccccgttcgccagagtctctcagaatttgaagatgcacgcctgggtcgtggaaaagccgaaacagcacaagctgctatccaacgcattttcccatcgattcaagcaactgctcatcgtctcactgttccaatgcctggacattctatcgatgaaaaagatgtaccagagctggaaaaagatattgcaaaattggagcaacttgtgaaagatcatgatgttgttttcctagcattagattcaagggaagctcgctggcttccaacagttttggctagtagacataagaagattgcaattagtgtcgcgattggattcgatacctacgtcatcattcggcatggaattggttctagaagcgaaagtgtttcagatgtttcatcatcagattccgtaccatactctcagctttcttgctatttctgcagtgatgtaactgctccaggaaattctactttcgatagaacattggaccaacagtgcactgttgcacgaccaggaacatctatgattgcatctggaattgccgttgaactcttatcgtctgttttacaataccctgatccactcaagacaccagccagccacgacgataatacaacagtccttggagctgctccacatcaaattcgcggttttctgggaagattccagcagattcttccttctgtaaaacgattcgatcaatgtgttgcttgtggagacgctatcgctgcacagtttcagcaaaatggctggaagtttgttcgcgacgttatgaactcgccaggtcgtctcgaagaagtcaccgggctcgacgagcttcagaactctgttaatgctattgatatcgattttgaggatgatgaagatttctga

SEQ ID NO: 42 - protein, 647 aa, Caenorhabditis elegans
MATFVPFVTCLDTGFWNEVNKKKLNDWKLDETPKCISSQLSLHQTEGFKCHLSLSYDSLSSLESTTGLSMSGTLLLYNTIESFKMVDKSDLIRSEAEKIWESITTRKWLQNPRLLSQFFIIAFADLKKFKYYYWTCVPALVYPSEIKQEITPLSSLGADHKILFDFYRKNNFPIFLYSKQSSKMLELSELENNTNPDEICVVVADPSPVAYSAGWMVRNVLAAVAHLHPTWKHCHIISLRSADSIGIKYTWTLPSAECSADGAQNAVPKAVGWERNANDKLQPISVDLSKEFDPKILMERSVDLNLSLIKWRLHPDIQLERYSQLKVLILGAGTLGCNIARCLIGWGVRHISFLDNSTVSYNNPVRQSLSEFEDARLGRGKAETAQAAIQRIFPSIQATAHRLTVPMPGHSIDEKDVPELEKDIAKLEQLVKDHDVVFLALDSREARWLPTVLASRHKKIAISVAIGFDTYVIIRHGIGSRSESVSDVSSSDSVPYSQLSCYFCSDVTAPGNSTFDRTLDQQCTVARPGTSMIASGIAVELLSSVLQYPDPLKTPASHDDNTTVLGAAPHQIRGFLGRFQQILPSVKRFDQCVACGDAIAAQFQQNGWKFVRDVMNSPGRLEEVTGLDELQNSVNAIDIDFEDDEDF

SEQ ID NO: 43 - DNA, 2112 bp, Homo sapiens
  CDS (1)..(2112)
atggcggcagctacgggggatcctggactctctaaactgcagtttgccccttttagtagtgccttggatgttgggttttggcatgagttgacccagaagaagctgaacgagtatcggctggatgaagctcccaaggacattaagggttattactacaatggtgactctgctgggctgccagctcgcttaacattggagttcagtgcttttgacatgagtgctcccaccccagcccgttgctgcccagctattggaacactgtataacaccaacacactcgagtctttcaagactgcagataagaagctccttttggaacaagcagcaaatgagatatgggaatccataaaatcaggcactgctcttgaaaaccctgtactcctcaacaagttcctcctcttgacatttgcagatctaaagaagtaccacttctactattggttttgctatcctgccctctgtcttccagagagtttacctctcattcaggggccagtgggtttggatcaaaggttttcactaaaacagattgaagcactagagtgtgcatatgataatctttgtcaaacagaaggagtcacagctcttccttacttcttaatcaagtatgatgagaacatggtgctggtttccttgcttaaacactacagtgatttcttccaaggtcaaaggacgaagataacaattggtgtatatgatccctgtaacttagcccagtaccctggatggcctttgaggaattttttggtcctagcagcccacagatggagtagcagtttccagtctgttgaagttgtttgcttccgtgaccgtaccatgcagggggcgagagacgttgcccacagcatcatcttcgaagtgaagcttccagaaatggcatttagcccagattgtcctaaagcagttggatgggaaaagaaccagaaaggaggcatgggaccaaggatggtgaacctcagtgaatgtatggaccctaaaaggttagctgagtcatcagtggatctaaatctcaaactgatgtgttggagattggttcctactttagacttggacaaggttgtgtctgtcaaatgtctgctgcttggagccggcaccttgggttgcaatgtagctaggacgttgatgggttggggcgtgagacacatcacatttgtggacaatgccaagatctcctactccaatcctgtgaggcagcctctctatgagtttgaagattgcctagggggtggtaagcccaaggctctggcagcagcggaccggctccagaaaatattccccggtgtgaatgccagaggattcaacatgagcatacctatgcctgggcatccagtgaacttctccagtgtcactctggagcaagcccgcagagatgtggagcaactggagcagctcatcgaaagccatgatgtcgtcttcctattgatggacaccagggagagccggtggcttcctgccgtcattgctgcaagcaagagaaagctggtcatcaatgctgctttgggatttgacacatttgttgtcatgagacatggtctgaagaaaccaaagcagcaaggagctggggacttgtgtccaaaccaccctgtggcatctgctgacctcctgggctcatcgctttttgccaacatccctggttacaagcttggctgctacttctgcaatgatgtggtggccccaggagattcaaccagagaccggaccttggaccagcagtgcactgtgagtcgtccaggactggccgtgattgcaggagccctggccgtggaattgatggtatctgttttgcagcatccagaagggggctatgccattgccagcagcagtgacgatcggatgaatgagcctccaacctctcttgggcttgtgcctcaccagatccggggatttctttcacggtttgataatgtccttcccgtcagcctggcatttgacaaatgtacagcttgttcttccaaagttcttgatcaatatgaacgagaaggatttaacttcctagccaaggtgtttaattcttcacattccttcttagaagacttgactggtcttacattgctgcatcaagaaacccaagctgctgagatctgggacatgagcgatgatgagaccatctga

SEQ ID NO: 44 - protein, 703 aa, Homo sapiens
MAAATGDPGLSKLQFAPFSSALDVGFWHELTQKKLNEYRLDEAPKDIKGYYYNGDSAGLPARLTLEFSAFDMSAPTPARCCPAIGTLYNTNTLESFKTADKKLLLEQAANEIWESIKSGTALENPVLLNKFLLLTFADLKKYHFYYWFCYPALCLPESLPLIQGPVGLDQRFSLKQIEALECAYDNLCQTEGVTALPYFLIKYDENMVLVSLLKHYSDFFQGQRTKITIGVYDPCNLAQYPGWPLRNFLVLAAHRWSSSFQSVEVVCFRDRTMQGARDVAHSIIFEVKLPEMAFSPDCPKAVGWEKNQKGGMGPRMVNLSECMDPKRLAESSVDLNLKLMCWRLVPTLDLDKVVSVKCLLLGAGTLGCNVARTLMGWGVRHITFVDNAKISYSNPVRQPLYEFEDCLGGGKPKALAAADRLQKIFPGVNARGFNMSIPMPGHPVNFSSVTLEQARRDVEQLEQLIESHDVVFLLMDTRESRWLPAVIAASKRKLVINAALGFDTFVVMRHGLKKPKQQGAGDLCPNHPVASADLLGSSLFANIPGYKLGCYFCNDVVAPGDSTRDRTLDQQCTVSRPGLAVIAGALAVELMVSVLQHPEGGYAIASSSDDRMNEPPTSLGLVPHQIRGFLSRFDNVLPVSLAFDKCTACSSKVLDQYEREGFNFLAKVFNSSHSFLEDLTGLTLLHQETQAAEIWDMSDDETI

SEQ ID NO: 45 - DNA, 288 bp, Caenorhabditis elegans
  CDS (1)..(288)
atgtctttggtgccagcttcattctatcaaaatgtggatattattcgagatgtgaagtttaatatgaagccaacgtgttcgatttgtatggaagaattcgatgcaaattctcatattccgaaggttctcatttgtggccactcgttttgcattatttgcatcgaaaaacatttgagatcatcaaatctcacattctggcgatggggatgttttcagtcattatacatgctctacttgccgcacaacgatggaaattcctgtcaacggagcagctggattttccactaa

SEQ ID NO: 46 - protein, 95 aa, Caenorhabditis elegans
MSLVPASFYQNVDIIRDVKFNMKPTCSICMEEFDANSHIPKVLICGHSFCIICIEKHLRSSNLTFWRWGCFQSLYMLYLPHNDGNSCQRSSWIFH

SEQ ID NO: 47 - DNA, 1962 bp, Homo sapiens
  CDS (1)..(1962)
atggctgcagcagcagcttctcacctgaacctggatgccctccgggaagtgctagaatgccccatctgcatggagtccttcacagaagagcagctgcgtcccaagcttctgcactgtggccataccatctgccgccagtgcctggagaagctattggccagtagcatcaatggtgtccgctgtcccttttgcagcaagattacccgcataaccagcttgacccagctgacagacaatctgacagtgctaaagatcattgatacagctgggctcagcgaggctgtggggctgctcatgtgtcggtcctgtgggcggcgtctgccccggcaattctgccggagctgtggtttggtgttatgtgagccctgccgggaggcagaccatcagcctcctggccactgtacactccctgtcaaagaagcagctgaggagcggcgtcgggactttggagagaagttaactcgtctgcgggaacttatgggggagctgcagcggcggaaggcagccttggaaggtgtctccaaggaccttcaggcaaggtataaagcagttctccaggagtatgggcatgaggagcgcagggtccaggatgagctggctcgctctcggaagttcttcacaggctctttggctgaagttgagaagtccaatagtcaagtggtagaggagcagagttacctgcttaacattgcagaggtgcaggctgtgtctcgctgtgactacttcctggccaagatcaagcaggcagatgtagcactactggaggagacagctgatgaggaggagccagagctcactgccagcttgcctcgggagctcaccctgcaagatgtggagctccttaaggtaggtcatgttggccccctccaaattggacaagctgttaagaagccccggacagttaacgtggaagattcctgggccatggaggccacagcgtctgctgcctctacctctgttacttttagagagatggacatgagcccggaggaagtggttgccagccctagggcctcacctgctaaacagcggggtcctgaggcagcctccaatatccagcagtgcctctttctcaagaagatgggggccaaaggcagcactccaggaatgttcaatcttccagtcagtctctacgtgaccagtcaaggtgaagtactagtcgctgaccgtggtaactatcgtatacaagtctttacccgcaaaggctttttgaaggaaatccgccgcagccccagtggcattgatagctttgtgctaagcttccttggggcagatctacccaacctcactcctctctcagtggcaatgaactgccaggggctgattggtgtgactgacagctatgataactccctcaaggtatataccttggatggccactgcgtggcctgtcacaggagccagctgagcaaaccatggggtatcacagccttgccatctggccagtttgtagtaaccgatgtggaaggtggaaagctttggtgtttcacagttgatcgaggatcaggggtggtcaaatacagctgcctatgtagtgctgtgcggcccaaatttgtcacctgtgatgctgagggcaccgtctacttcacccagggcttaggcctcaatctggagaatcggcagaatgagcaccacctggagggtggcttttccattggctctgtaggccctgatgggcagctgggtcgccagattagccacttcttctcggagaatgaggatttccgctgcattgctggcatgtgtgtggatgctcgtggtgatctcatcgtggctgacagtagtcgcaaggaaattctccattttcctaagggtgggggctatagtgtccttattcgagagggacttacctgtccggtgggcatagccctaactcctaaggggcagctgctggtcttggactgttgggatcattgcatcaagatctacagctaccatctgagaagatattccaccccatag

SEQ ID NO: 48 - protein, 653 aa, Homo sapiens
MAAAAASHLNLDALREVLECPICMESFTEEQLRPKLLHCGHTICRQCLEKLLASSINGVRCPFCSKITRITSLTQLTDNLTVLKIIDTAGLSEAVGLLMCRSCGRRLPRQFCRSCGLVLCEPCREADHQPPGHCTLPVKEAAEERRRDFGEKLTRLRELMGELQRRKAALEGVSKDLQARYKAVLQEYGHEERRVQDELARSRKFFTGSLAEVEKSNSQVVEEQSYLLNIAEVQAVSRCDYFLAKIKQADVALLEETADEEEPELTASLPRELTLQDVELLKVGHVGPLQIGQAVKKPRTVNVEDSWAMEATASAASTSVTFREMDMSPEEVVASPRASPAKQRGPEAASNIQQCLFLKKMGAKGSTPGMFNLPVSLYVTSQGEVLVADRGNYRIQVFTRKGFLKEIRRSPSGIDSFVLSFLGADLPNLTPLSVAMNCQGLIGVTDSYDNSLKVYTLDGHCVACHRSQLSKPWGITALPSGQFVVTDVEGGKLWCFTVDRGSGVVKYSCLCSAVRPKFVTCDAEGTVYFTQGLGLNLENRQNEHHLEGGFSIGSVGPDGQLGRQISHFFSENEDFRCIAGMCVDARGDLIVADSSRKEILHFPKGGGYSVLIREGLTCPVGIALTPKGQLLVLDCWDHCIKIYSYHLRRYSTP

SEQ ID NO: 49 - DNA, 1125 bp, Caenorhabditis elegans
  CDS (1)..(1125)
atggggcaagcgaagttgcggctgacgcgttattggatgctggcgcgtttcgcgaccagcgttggttttatcgagaattttctctgcagtacaaagtcccaaattcggtggttttttatcgatttgacgcgcgtttgctcaatttctcgattttccgcgttttttattcagttctcattaattaacgttcgatgcttgttcacaaaattcagtttttgttttcacttgctcgttggtgtcgttcgttgtaatatgtttggaggtggaagtagtggtcccgtggacaccactttatacacaacactcaatgtgagaccagacgcttcgcaggccgacattaagaaatcttacttcaaacttgctaaagaataccatccagataaaaacccggaccatggagataaattcaaagagatcagttttgcctatgaagttctttcgagccctgaaaaacgacgcttgtatgacgccagaggtttggaaggagttcaaggaggaggagctggtggtggtggaggaggctttcctggaggtctgttctctcacttcttcggcggtgctggcggtgatgacgatgacgacgatgatgatatgggtggtcatccatttggtggcttgttcggtggaatgggtggaatgggacgaggtggcccacgtcggcggaaattccaagatactgttcatcccctcaatgttacactcgaagagctttacgtcggaaaaacatcaaagctgaagctttccaaaaaggcactctgtaaaacttgcgaagggtcaggtggaaagaagggagaaaaatataagtgtgatgcatgccgtggtcgtggagtgaagacgatcgttcagcaaattggccccggaatgctccaacaaatgcaggttcactgtgatgcttgtaagggttctggaggcaaagttccagcaggtgataagtgcaaaggatgccatggagaaaagtacgaaaacgtttcgaaaatattggaggttcacgttcttcctggcatgaaacataacgataaaattacattcaaaggagatggagaccaatctgacccagatggtgagccaggagatgttgtcattgttattcaacagaaagatccatctctgatatgttctgaatga

SEQ ID NO: 50 - protein, 374 aa, Caenorhabditis elegans
MGQAKLRLTRYWMLARFATSVGFIENFLCSTKSQIRWFFIDLTRVCSISRFSAFFIQFSLINVRCLFTKFSFCFHLLVGVVRCNMFGGGSSGPVDTTLYTTLNVRPDASQADIKKSYFKLAKEYHPDKNPDHGDKFKEISFAYEVLSSPEKRRLYDARGLEGVQGGGAGGGGGGFPGGLFSHFFGGAGGDDDDDDDDMGGHPFGGLFGGMGGMGRGGPRRRKFQDTVHPLNVTLEELYVGKTSKLKLSKKALCKTCEGSGGKKGEKYKCDACRGRGVKTIVQQIGPGMLQQMQVHCDACKGSGGKVPAGDKCKGCHGEKYENVSKILEVHVLPGMKHNDKITFKGDGDQSDPDGEPGDVVIVIQQKDPSLICSE

SEQ ID NO: 51 - DNA, 1239 bp, Homo sapiens
  CDS (1)..(1239)
atggctaacgtggctgacacgaagctgtacgacatcctgggcgtcccgcccggcgccagcgagaacgagctgaagaaggcatacagaaagttagccaaggaatatcatcctgataagaatccaaatgcaggagacaaatttaaagaaataagttttgcatatgaagtactatcaaatcctgagaagcgtgagttatatgacagatacggagagcaaggtcttcgggaaggcagcggcggaggtggtggcatggatgatattttctctcacatttttggtgggggattgttcggcttcatgggcaatcagagtagaagtcgaaatggcagaagaagaggagaggacatgatgcatccactcaaagtatctttagaagatctgtataatggcaagacaaccaaactacaacttagcaagaatgtgctctgtagtgcatgcagtggccaaggcggaaagtctggagctgtccaaaagtgtagtgcttgtcgaggtcgaggtgtgcgcatcatgatcagacagctggctccagggatggtacaacagatgcagtctgtgtgctctgattgtaatggagaaggagaggtaattaatgaaaaagaccgctgtaaaaaatgtgaagggaagaaggtgattaaagaagtcaagattcttgaagtccacgtagacaaaggcatgaaacatggacagagaattacattcactggggaagcagaccaggccccaggagtggaacccggagacattgttcttttgctacaggagaaagaacatgaggtatttcagagagatgggaatgatttgcacatgacatataaaataggacttgttgaagctctatgtggatttcagttcacatttaagcaccttgatggacgtcagattgtggtgaaatacccccctggcaaagtaattgaaccagggtgtgttcgtgtagttcgaggtgaagggatgccgcagtatcgtaatccctttgaaaaaggtgatctttacataaagtttgatgtgcagtttcctgaaaacaactggatcaacccagacaagctttctgaactagaagatcttctgccatctagaccggaagttcctaacataattggagaaacagaggaggtagagcttcaggaatttgatagcactcgaggctcaggaggtggtcagaggcgtgaagcctataatgatagctctgatgaagaaagcagcagccatcatggacctggagtgcagtgtgcccatcagtaa

SEQ ID NO: 52 - protein, 412 aa, Homo sapiens
MANVADTKLYDILGVPPGASENELKKAYRKLAKEYHPDKNPNAGDKFKEISFAYEVLSNPEKRELYDRYGEQGLREGSGGGGGMDDIFSHIFGGGLFGFMGNQSRSRNGRRRGEDMMHPLKVSLEDLYNGKTTKLQLSKNVLCSACSGQGGKSGAVQKCSACRGRGVRIMIRQLAPGMVQQMQSVCSDCNGEGEVINEKDRCKKCEGKKVIKEVKILEVHVDKGMKHGQRITFTGEADQAPGVEPGDIVLLLQEKEHEVFQRDGNDLHMTYKIGLVEALCGFQFTFKHLDGRQIVVKYPPGKVIEPGCVRVVRGEGMPQYRNPFEKGDLYIKFDVQFPENNWINPDKLSELEDLLPSRPEVPNIIGETEEVELQEFDSTRGSGGGQRREAYNDSSDEESSSHHGPGVQCAHQ

SEQ ID NO: 53 - DNA, 867 bp, Caenorhabditis elegans
  CDS (1)..(867)
atgcccgacttcaccaacaagcaaattatattagatgaaaaatacgagtatatggattcagtggacgccatgattgggatgggagcatttggagcagtgttcaaggggagcttgaaggattctagcaattcgattgccataaaaaggatgctcaaagtgcacgtgaaggagagcgaattgaaaatgatcaaagaactcaacagtgaatatctagtcggagtactagacatttgcaatttcgacgactttttctgctgtttaataatggaactctgcgattgtgatcttgaccatcacatgcgcaatatttcggtcaaaggaagattgaatccgtcgaatttcaggcttcttcttgacaacattgcccgaggatacaaagcgctttatgagttgaaaattgtacatcgggacattaaaccccaaaacatactgatcacttatacagatgcatcgaaacaaattgcttgtgctcggatcacagactttggaatttcgagaacccttgataacgagggagaagagctgtgcaacgttgcgggtacattttattacatggctccagaagttggagcgaaccttttaaaaacttgtcagtatgactcaaaagttgatatgtggagcatcggatgccttctctaccaatgcgttacaggagaggtcccattcgatgagtgcagtttgtgtaagctcttcctgtatgtggcaggggctaacttcgacgcctacgacccgcctgagctgcccgacgagctgagtcaagaggtttcggggattattcaatcactgctgcagctcgataccactcaaagatgcacacctactcagttttatgacaaagcaatcaactggagtcaacaaatatgttag

SEQ ID NO: 54 - protein, 288 aa, Caenorhabditis elegans
MPDFTNKQIILDEKYEYMDSVDAMIGMGAFGAVFKGSLKDSSNSIAIKRMLKVHVKESELKMIKELNSEYLVGVLDICNFDDFFCCLIMELCDCDLDHHMRNISVKGRLNPSNFRLLLDNIARGYKALYELKIVHRDIKPQNILITYTDASKQIACARITDFGISRTLDNEGEELCNVAGTFYYMAPEVGANLLKTCQYDSKVDMWSIGCLLYQCVTGEVPFDECSLCKLFLYVAGANFDAYDPPELPDELSQEVSGIIQSLLQLDTTQRCTPTQFYDKAINWSQQIC

SEQ ID NO: 55 - DNA, 3111 bp, Homo sapiens
  CDS (1)..(3111)
atggaggtggtgggtgacttcgagtacagcaagagggatctcgtgggacacggggccttcgccgtggtcttccgggggcggcaccgccagaaaactgattgggaggtagctattaaaagtattaataaaaagaacttgtcaaaatcacaaatactgcttggaaaggaaattaaaatcttaaaggaacttcagcatgaaaatattgtagcactctatgatgttcaggaattacccaactctgtctttttggtgatggagtattgcaatggtggagacctcgcagattatttgcaagcgaaagggactctcagtgaagacacgatcagagtgtttctgcatcagattgctgctgccatgcgaatcctgcacagcaaaggaatcatccacagagatctcaaaccacagaacatcttgctgtcctatgccaatcgcagaaaatcaagtgtcagtggtattcgcatcaaaatagcggattttggttttgctcgttacctacatagtaacatgatggctgcaacactgtgtggatccccgatgtacatggctcctgaggttattatgtctcaacattatgatgctaaggctgacttgtggagcataggaacagtgatataccaatgcctagttggaaaaccaccttttcaggccaatagtcctcaagacttaaggatgttttatgaaaaaaacaggagcttaatgcctagtattcccagagaaacatcaccttatttggctaatctccttttgggtttgcttcagagaaaccaaaaagatagaatggactttgaagcattttttagccatccttttcttgagcaaggtccagtaaaaaaatcttgcccagttccagtgcccatgtattctggttctgtctctggaagctcctgtggcagctctccatcttgtcgttttgcttctccaccatcccttccagatatgcagcatattcaggaagaaaacttatcttccccaccattgggtcctcccaactatctacaagtttccaaagattctgccagtactagtagcaagaactcttcttgtgacacggatgactttgttttggtgccacacaacatctcgtcagaccactcatgtgatatgccagtggggactgctggcagacgtgcttcaaatgaattcttggtgtgtggagggcagtgtcagcctactgtgtcacctcacagcgaaacagcaccaattccagttcctactcaaataaggaattatcagcgcatagagcagaatcttacatctactgccagctcaggcacaaatgtacatggttctccaagatctgcagtggtacgaaggtccaacaccagccccatgggcttcctccggccgggatcatgctccccagtaccagcagacacagcacagacagttggacgaaggctctccactgggtcttctaggccttactcaccttcccctttggttggtaccattcctgagcaattcagtcagtgctgctgtgggcatcctcagggccatgactccaggagtagaaactcctcaggttctccagtgccacaagctcagtccccacagtctctcttatcgggtgctagactgcagagcgcccccaccctcactgacatctatcagaacaagcagaagctcagaaaacagcactctgaccccgtgtgcccatcccatactggggctgggtacagctactcgcctcagcccagtcggcctggcagccttggaacttctcccaccaagcacttggggtcctctccacggagttctgactggttctttaaaactcctttgccaacaatcattggctctcctactaagaccacagctcctttcaaaatccctaaaactcaagcatcttccaacctgttagccttggttactcgtcatgggcctgctgaagaacagtcgaaagatgggaatgagccacgggaatgtgcccattgcctcttagtgcaaggaagtgagaggcagcgggccgagcagcagagcaaggcagtgtttggcagatctgtcagtaccgggaagttatcagatcaacaaggaaagactcctatatgtcgacatcagggcagcacagacagtttaaatacagaacgaccaatggatatagctccggcaggagcctgtggtggtgttctggcacctcctgcaggtacagcagcaagttccaaggctgtcctcttcactgtagggtctcctccacacagtgcggcagcccccacttgtacccacatgttccttcgaacaagaacaacctcagtggggcccagcaactccgggggctctctttgtgccatgagtggccgcgtgtgcgtggggtccccgcctggcccaggcttcggctcttcccctccaggagcagaggcagctcccagcctgagatacgtgccttacggtgcttcaccccccagcctagaggggctcatcacctttgaagcccctgaactgccggaggagacgctgatggagcgggaacacacagacaccttacgccatctgaatgtgatgctgatgttcactgagtgtgtgctggacctgacagccatgaggggaggaaaccctgagctgtgcacatctgctgtgtccttgtaccagatccaggagagtgtggtggtggaccagatcagtcagctgagcaaagactgggggcgggtggagcagctggtgttgtacatgaaagcagcacagctgcttgcggcttctctgcatcttgccaaagcccagatcaagtccgggaaactgagcccatccacagctgtgaaacaagttgtcaagaatctgaacgaacgatataaattctgcatcaccatgtgcaagaaacttacagaaaagctgaatcgattcttctctgacaaacagaggtttattgatgaaatcaacagtgtgactgcagagaaactcatctataattgtgctgtagaaatggttcagtctgcagccctggatgagatgtttcagcagaccgaagatattgtttatcgctatcataaggcagcccttcttttggaaggcctaagtaggattctacaggaccctgcagatattgaaaatgtgcataaatataaatgtagtattgagagaagactgtcggcgctctgccatagcaccgcaaccgtgtga

SEQ ID NO: 56 - protein, 1036 aa, Homo sapiens
MEVVGDFEYSKRDLVGHGAFAVVFRGRHRQKTDWEVAIKSINKKNLSKSQILLGKEIKILKELQHENIVALYDVQELPNSVFLVMEYCNGGDLADYLQAKGTLSEDTIRVFLHQIAAAMRILHSKGIIHRDLKPQNILLSYANRRKSSVSGIRIKIADFGFARYLHSNMMAATLCGSPMYMAPEVIMSQHYDAKADLWSIGTVIYQCLVGKPPFQANSPQDLRMFYEKNRSLMPSIPRETSPYLANLLLGLLQRNQKDRMDFEAFFSHPFLEQGPVKKSCPVPVPMYSGSVSGSSCGSSPSCRFASPPSLPDMQHIQEENLSSPPLGPPNYLQVSKDSASTSSKNSSCDTDDFVLVPHNISSDHSCDMPVGTAGRRASNEFLVCGGQCQPTVSPHSETAPIPVPTQIRNYQRIEQNLTSTASSGTNVHGSPRSAVVRRSNTSPMGFLRPGSCSPVPADTAQTVGRRLSTGSSRPYSPSPLVGTIPEQFSQCCCGHPQGHDSRSRNSSGSPVPQAQSPQSLLSGARLQSAPTLTDIYQNKQKLRKQHSDPVCPSHTGAGYSYSPQPSRPGSLGTSPTKHLGSSPRSSDWFFKTPLPTIIGSPTKTTAPFKIPKTQASSNLLALVTRHGPAEEQSKDGNEPRECAHCLLVQGSERQRAEQQSKAVFGRSVSTGKLSDQQGKTPICRHQGSTDSLNTERPMDIAPAGACGGVLAPPAGTAASSKAVLFTVGSPPHSAAAPTCTHMFLRTRTTSVGPSNSGGSLCAMSGRVCVGSPPGPGFGSSPPGAEAAPSLRYVPYGASPPSLEGLITFEAPELPEETLMEREHTDTLRHLNVMLMFTECVLDLTAMRGGNPELCTSAVSLYQIQESVVVDQISQLSKDWGRVEQLVLYMKAAQLLAASLHLAKAQIKSGKLSPSTAVKQVVKNLNERYKFCITMCKKLTEKLNRFFSDKQRFIDEINSVTAEKLIYNCAVEMVQSAALDEMFQQTEDIVYRYHKAALLLEGLSRILQDPADIENVHKYKCSIERRLSALCHSTATV

SEQ ID NO: 57 - DNA, 2280 bp, Caenorhabditis elegans
  CDS (1)..(2280)
atgccaaagtctatatatttttcccctgactttatccgacagcaatccggccactgcttcatcatgggagagcggaacctggttctttatgagaaaagaatgtttcaatacaaagcttcgagtctgtactctggatccgaaagagatggttttatccactgttgttcttggaatgagaatctcattgcgttcacaaatgataccggaacaagagtctacgaaagaggcgcagagagaatcatcacaagtgttcagccatcacatgacgtggatcgcgtccgatcatctcgctcaccaccgaaacacacttggatgcctgagaacaacttagtgattggttgggcagacacggttaccatcctcaaaattcgtgatgatgatggagtaaaaaaaggagaggttcatcatattttccacgtatcaatgttcatttgtggaatctcctacatcccggaaagcggcatcgacaatatggagctgttcctagttgggttgcagttggaaggggaggactttgatgattgtgcgtcagttatttccactgtgacaacattgaccgcattggaaagtagtgcatgcacaatcctgaagacgtctgtgatccggccacttggattgaaagaatttgagcttcagtcagaggatatgattgaaagcgtcaaactctccaaccatactttgccatacatgattcacggtcttggaattccttaccttgccacatactttatattgacaacgaagcacattattatggcggtaccatatggcccggaagacggcattcgttggagattgaagtataaactctacgatgaagcgttggatatggcaaagcacaatgccgacttactctccaaaactgatctcagtccgaagaaagtggggaggatgattatcgagggatacctgactggaaaacgagcaagagcagctgcttcccgccttccattgatctgtggagaatgcaaggaggagtgggaatgggcagtgaatcagtttgaggaagtcaaattatgcactctactagcggaggtcctgcccgatggcactccgacattggatccagagtgctaccagaaagttcttattgcttgtctgttcaacaatgtgaagcagttccggaaattggtacagacgtggagtccggatctctatatgaccagttttataatcgatcggactcaatggcgcattcaacaaatcagcaaatcgggcaatctagcagacgttgacgagactgagcgagttttgatggacgctttggcacatttgtatctctacgagagaaagtacgagagcgcactgaaaatccttatgtcgtgtcaagattttcaaattttcaatgttattgacaagcatcaactcttcgatcttgtcaaggatcaaatcaccgaactgatgaacatcaactctgaacgtgctcttcggctgttgctcgacaacgctgattcggtggagccatcatttgtgatggagaaaattgggcgacagccgaaactgcaactcgcgtatctcacaaaactgatgagcagaaacgagggaactgagtttgctgacaaagctgtccagttgtatgctgaatacgaccagaagaagcttcttccatttttgagaaagaatgcaaactacaatgtgaacaaggcacgaaagttgtgctcggataagggatatattgaagagacaatctatcttcttgccaaaagtggaaatcattatgatgctgtgaaaatgatggttcgagagtatcggaacatggaaaaagtcatcgattactgcaaagatcaaaatgaccccgatttatggattcaccttctaggagtagttgccgagtttcctgctcatttttcgcagcttatcattgaagcgtcaaactgcctcgatcctctcctgattatggacaaactaccggacgattcggatattcctaacttgagtgaagcgctcgacaaacttctcgttgactatacaaatcatgcagagcttcagcaatgttgctacgactcaaccttgaacgatctgaatgttctcactcaaggactgatatcggcagctgatgaatcagtttccgtgaacatagtatccagatgttcattatgtgctcaaatcatcatcaattctaaccaagaaacaacaaaaaagttttcggatataaaagtgttcaaatgcggacacatttttcacttggcttgttctacttctgagatggaacgccgtcaatcaattgaggagggcctctgcattgcgtgctctgatcaaatcgaactcatcaacgtgtaa

SEQ ID NO: 58 - protein, 759 aa, Caenorhabditis elegans
MPKSIYFSPDFIRQQSGHCFIMGERNLVLYEKRMFQYKASSLYSGSERDGFIHCCSWNENLIAFTNDTGTRVYERGAERIITSVQPSHDVDRVRSSRSPPKHTWMPENNLVIGWADTVTILKIRDDDGVKKGEVHHIFHVSMFICGISYIPESGIDNMELFLVGLQLEGEDFDDCASVISTVTTLTALESSACTILKTSVIRPLGLKEFELQSEDMIESVKLSNHTLPYMIHGLGIPYLATYFILTTKHIIMAVPYGPEDGIRWRLKYKLYDEALDMAKHNADLLSKTDLSPKKVGRMIIEGYLTGKRARAAASRLPLICGECKEEWEWAVNQFEEVKLCTLLAEVLPDGTPTLDPECYQKVLIACLFNNVKQFRKLVQTWSPDLYMTSFIIDRTQWRIQQISKSGNLADVDETERVLMDALAHLYLYERKYESALKILMSCQDFQIFNVIDKHQLFDLVKDQITELMNINSERALRLLLDNADSVEPSFVMEKIGRQPKLQLAYLTKLMSRNEGTEFADKAVQLYAEYDQKKLLPFLRKNANYNVNKARKLCSDKGYIEETIYLLAKSGNHYDAVKMMVREYRNMEKVIDYCKDQNDPDLWIHLLGVVAEFPAHFSQLIIEASNCLDPLLIMDKLPDDSDIPNLSEALDKLLVDYTNHAELQQCCYDSTLNDLNVLTQGLISAADESVSVNIVSRCSLCAQIIINSNQETTKKFSDIKVFKCGHIFHLACSTSEMERRQSIEEGLCIACSDQIELINV

SEQ ID NO: 59 - DNA, 2565 bp, Homo sapiens
  CDS (1)..(2565)
atggcggaagcagaggagcaggaaactgggtcccttgaagaatctacagatgagtctgaggaagaagagagcgaagaggaacccaagctgaagtatgaaaggctttccaatggggtaactgaaatacttcagaaggatgcagctagctgcatgacagtccatgacaagtttttggcattgggcacacattatggcaaggtttatttacttgatgtccaggggaacatcactcagaagtttgatgtaagtcctgtgaagataaatcagattagcttggatgaaagtggagagcacatgggtgtgtgttcagaggatggcaaggtgcaggtatttggactgtattctggagaagaatttcacgagacttttgactgtcccattaaaattattgctgtgcacccacatttcgtgagatccagttgcaagcagtttgtgaccggagggaagaagctgctactgtttgaacggtcttggatgaacagatggaagtctgctgttctgcatgaaggggaagggaacataaggagtgtgaagtggagaggccatctgattgcttgggccaataatatgggtgtgaagatttttgacatcatctcaaagcaaagaatcaccaatgtgccccgggatgatataagtcttcgcccagacatgtatccctgcagcctctgctggaaggacaatgtgacactgattattggctgggggacttctgtcaaggtgtgctcagtgaaggaacggcatgccagtgaaatgagggatttgccaagtcgatatgttgaaatagtgtctcagtttgaaactgaattctacatcagtggacttgcacctctctgtgatcagcttgttgtactttcgtatgtaaaggagatttcagaaaaaacggaaagagaatactgtgccaggcctagactggacatcatccagccactttctgagacttgtgaagagatctcttctgatgctttgacagtcagaggctttcaggagaatgaatgtagagattatcatttagaatactctgaaggggaatcacttttttacatcgtgagtccgagagatgttgtagtggccaaggaacgagaccaagatgatcacattgactggctccttgaaaagaagaaatatgaagaagcattgatggcagctgaaattagccaaaaaaatattaaaagacataagattctggatattggcttggcatatataaatcacctggtggagagaggagactatgacatagcagcacgcaaatgccagaaaattcttgggaaaaatgcagcactctgggaatatgaagtttataaatttaaagaaattggacagcttaaggctattagtccttatttgccaagaggtgatccagttctgaaaccactcatctatgaaatgatcttacatgaatttttggagagtgattatgagggttttgccacattgatccgagaatggcctggagatctgtataataattcagtcatagttcaagcagttcgggatcatttgaagaaagatagtcagaacaagactttacttaaaaccctggcagaattgtacacctatgacaagaactatggcaatgctctggaaatatacttaacattaagacataaagacgtttttcagttgatccacaagcataatcttttcagttctatcaaggataaaattgttttattaatggattttgattcagagaaagctgttgacatgcttttggacaatgaagataaaatttcaattaaaaaggtagtggaagaattggaagacagaccagagctacagcatgtgtatttgcataagcttttcaagagagaccaccataaggggcagcgttaccatgaaaaacagatcagtctttatgctgaatatgatcgaccaaacttacttccctttctccgagacagtacccattgcccacttgaaaaggctcttgagatctgtcaacagagaaactttgtagaagagacagtttatcttctgagccgaatgggtaatagccgaagtgccctgaagatgattatggaggaattacatgatgttgataaagcaatcgaatttgccaaggagcaagatgatggagagctgtgggaagatttgattttatattccattgacaaaccaccatttattactggcttgttaaacaacattggcacacatgttgacccaattctactgattcaccgtattaaggaaggaatggagatccccaatttgagagattccttggttaaaattctgcaagactacaatttgcaaattctgcttcgtgaaggctgcaagaagattctcgtagctgactctttgtccttactgaagaaaatgcaccgaactcaaatgaaaggtgttcttgttgatgaggagaacatctgtgagtcgtgcctttcccctattcttccatcagatgcagctaagcccttcagcgtggtggtcttccattgccggcacatgttccacaaggagtgcctgcccatgcccagcatgaactctgctgcacagttctgcaacatctgcagtgctaagaaccgtggaccaggaagtgcaattttggagatgaaaaaatag

SEQ ID NO: 60 - protein, 854 aa, Homo sapiens
MAEAEEQETGSLEESTDESEEEESEEEPKLKYERLSNGVTEILQKDAASCMTVHDKFLALGTHYGKVYLLDVQGNITQKFDVSPVKINQISLDESGEHMGVCSEDGKVQVFGLYSGEEFHETFDCPIKIIAVHPHFVRSSCKQFVTGGKKLLLFERSWMNRWKSAVLHEGEGNIRSVKWRGHLIAWANNMGVKIFDIISKQRITNVPRDDISLRPDMYPCSLCWKDNVTLIIGWGTSVKVCSVKERHASEMRDLPSRYVEIVSQFETEFYISGLAPLCDQLVVLSYVKEISEKTEREYCARPRLDIIQPLSETCEEISSDALTVRGFQENECRDYHLEYSEGESLFYIVSPRDVVVAKERDQDDHIDWLLEKKKYEEALMAAEISQKNIKRHKILDIGLAYINHLVERGDYDIAARKCQKILGKNAALWEYEVYKFKEIGQLKAISPYLPRGDPVLKPLIYEMILHEFLESDYEGFATLIREWPGDLYNNSVIVQAVRDHLKKDSQNKTLLKTLAELYTYDKNYGNALEIYLTLRHKDVFQLIHKHNLFSSIKDKIVLLMDFDSEKAVDMLLDNEDKISIKKVVEELEDRPELQHVYLHKLFKRDHHKGQRYHEKQISLYAEYDRPNLLPFLRDSTHCPLEKALEICQQRNFVEETVYLLSRMGNSRSALKMIMEELHDVDKAIEFAKEQDDGELWEDLILYSIDKPPFITGLLNNIGTHVDPILLIHRIKEGMEIPNLRDSLVKILQDYNLQILLREGCKKILVADSLSLLKKMHRTQMKGVLVDEENICESCLSPILPSDAAKPFSVVVFHCRHMFHKECLPMPSMNSAAQFCNICSAKNRGPGSAILEMKK

SEQ ID NO: 61 - DNA, 1305 bp, Caenorhabditis elegans
  CDS (1)..(1305)
atgtactataaaatctcttatttattatctatcagtattctattatttttcgcgaaatgtgaaaaagtaaaaaataaggcagtggagaagaaagatgatcgaaatgaacgagaactggcaaagcacttacttgatgattactaccaatatactcgaccggttcggaattattccagtgtgctcaatgtgacggtacagccacaaatttacaacttggtagaggtgaatgaacaaaatgagcaaatcaaaatattgctctggttcccccaaagttggaaagacgattatctaacatgggatcctaaggaatggaacggaatagagagaataataatacccaaatcgcaaatttggattccagatggatatatatttaatacagtagaagaaactgaaccgttagaaaaccacaatgcaagagtaagatacgatgggcgagtggaagttgatttcaataaactcgtcgacttgacgtgccctatgtcagtgctctctttcccatttgatgttcaattatgtgccctccagtttggctcctggtcgtatcaagctcatgcgattagcttcaacgtactcgacacatttgtcccaaagaaaagcaagaattctgaatgggacattgtatcgtttaatgctacaaagatgacaacaaaatatggtgacacgcttggaggattcaatgtttatgaagagattttctattacttggaacttcgccgaaagccattgtattacattgtagtcattctgctcccatcatttttaattgttaccgtctcaaacattggactgtttacgcctcatggagttcacggagacagagaagagcatgtgtcattgggtctcactacaatgcttaccatggctgttatcttagacatggtcacaggacaaatgccaagaagtagtgaaggaataccactattgggaatgtatgtgctgattgaatttgttatatctgtaattgctgtattagtatcagttgtaataatttttgcacacgaaaggatgctatatctcgatgcaactcctccgtactgggtgtgcaaattgttttcagatgactgcaaaatgtcattggaagaaatagaagaagatttttgttcaaaaccagctgatctggtgcaagagttaaggttttgtatggaggaaatcaaacgatatcttgacgaacaggattcgacagagaagaaccgaattatatggcagaggtttttctcgtggacagatattatattcagtatattcttttttgttgttaattgtcttgtgacattttacatgtttatggagtttatgttttga

SEQ ID NO: 62 - protein, 434 aa, Caenorhabditis elegans
MYYKISYLLSISILLFFAKCEKVKNKAVEKKDDRNERELAKHLLDDYYQYTRPVRNYSSVLNVTVQPQIYNLVEVNEQNEQIKILLWFPQSWKDDYLTWDPKEWNGIERIIIPKSQIWIPDGYIFNTVEETEPLENHNARVRYDGRVEVDFNKLVDLTCPMSVLSFPFDVQLCALQFGSWSYQAHAISFNVLDTFVPKKSKNSEWDIVSFNATKMTTKYGDTLGGFNVYEEIFYYLELRRKPLYYIVVILLPSFLIVTVSNIGLFTPHGVHGDREEHVSLGLTTMLTMAVILDMVTGQMPRSSEGIPLLGMYVLIEFVISVIAVLVSVVIIFAHERMLYLDATPPYWVCKLFSDDCKMSLEEIEEDFCSKPADLVQELRFCMEEIKRYLDEQDSTEKNRIIWQRFFSWTDIIFSIFFFVVNCLVTFYMFMEFMF

SEQ ID NO: 63 - DNA, 1440 bp, Homo sapiens
  CDS (1)..(1440)
atgaactggtcccattcctgcatctccttttgctggatctactttgctgcttccagactgagagctgcagagacggcagatggaaaatatgctcagaagttgtttaatgacctttttgaagattattctaatgctcttcgtccagtggaagatacagataaagtcctgaatgtgaccctgcagattacgctctctcagattaaggatatggatgaaagaaaccaaattctgactgcttatttgtggatccgccaaatctggcacgatgcctatctcacgtgggaccgagatcagtacgatggcctagactccatcaggatccccagtgacctcgtgtggaggccagacatcgtcttatataacaaggctgatgatgaatcttcagagcctgtgaacaccaatgtggtcctgcggtatgatgggctgatcacctgggatgcaccggccatcaccaaaagctcctgtgtggtggatgtcacctacttcccttttgacaaccagcagtgcaacctgacttttggttcctggacctacaatggcaatcaggtggacatattcaacgccttggacagcggagatctctctgacttcattgaagatgtggaatgggaggtccatggcatgcccgctgtgaagaatgtgatctcctatggctgctgctctgagccttacccggatgtcacattcaccctccttctgaagaggaggtcctcgttctatatcgtcaacctcctcatcccatgcgtcctcatatcttttctggctcctctgagtttttatctcccagcagcctccggagaaaaggtctccctgggagtgaccatcctgttggccatgactgtatttcagctaatggtggcagaaatcatgccggcctcagaaaatgtgcccctgataggtaaatactacatagccacgatggccctgatcacagcctccactgcgttgaccatcatggtgatgaatatccacttctgtggggccgaggcccggccggtgccacactgggccagggtggtcatcctgaaatacatgtccagggtcttgtttgtctatgatgtgggtgaaagctgcctcagcccgcaccacagtagagagcgggaccacctcacgaaagtttatagcaaactcccagagtctaacctgaaagcagccaggaacaaagacctttccagaaagaaggacatgaacaaacgcttaaagaacgacctgggctgccagggtaagaaccctcaggaggccgagagttactgtgcacagtacaaagtgctgacgaggaatattgagtacatcgccaagtgcctcaaagaccacaaggccaccaattccaaggggagtgaatggaagaaggtggcgaaagtcatagaccgattcttcatgtggatttttttcattatggtgtttgtgatgactattttgatcatagcaagagcggattag

SEQ ID NO: 64 - protein, 479 aa, Homo sapiens
MNWSHSCISFCWIYFAASRLRAAETADGKYAQKLFNDLFEDYSNALRPVEDTDKVLNVTLQITLSQIKDMDERNQILTAYLWIRQIWHDAYLTWDRDQYDGLDSIRIPSDLVWRPDIVLYNKADDESSEPVNTNVVLRYDGLITWDAPAITKSSCVVDVTYFPFDNQQCNLTFGSWTYNGNQVDIFNALDSGDLSDFIEDVEWEVHGMPAVKNVISYGCCSEPYPDVTFTLLLKRRSSFYIVNLLIPCVLISFLAPLSFYLPAASGEKVSLGVTILLAMTVFQLMVAEIMPASENVPLIGKYYIATMALITASTALTIMVMNIHFCGAEARPVPHWARVVILKYMSRVLFVYDVGESCLSPHHSRERDHLTKVYSKLPESNLKAARNKDLSRKKDMNKRLKNDLGCQGKNPQEAESYCAQYKVLTRNIEYIAKCLKDHKATNSKGSEWKKVAKVIDRFFMWIFFIMVFVMTILIIARAD

SEQ ID NO: 65 - DNA, 1674 bp, Caenorhabditis elegans
  CDS (1)..(1674)
atggcatcgtctaataacgatggtcccattgagcctgaggctgaaccgtggcgaataacacaaaacgatcatctcgagcaagatcttttggaagaagatgcagagagtcaggagagagttgatattccagttgatgatgtggaaaaagcgttctccttcaaaaaattatgggcgttcacagggcccggatttctcatgagtattgcctatttggacccaggaaacattgaaagcgaccttcagtctggcgcacaagcggcttacaaacttttgtgggttttgctctcagctcatattatcggaatgttgctccaacgaatgtctgcaagacttggagttgtcagtggaaaacatatggcagaggttgcctaccaattttacccaagacttcctcgcatcattctctggcttatgatcgagattgcaattgtgtgtagtgatatgcaagaagttattggaacagcaattgccattttcttgctctctaaaggattcgttccactctacgttggagttttcatcacaattcttgacaccttcacatttttgctgatcgaccggtacggaatccgaaaactggaattaatctttggatttcttattttaacaatgacagtgtcatttggctatgagtttgtcgttgtgaaacctccaattggtgaagttatatctggaatggtggtaccttggtgcgctggatgtggaaaaggagagtttatgcaagcgatatcagttgtcggagctgtcatcatgccgcacaatttgtaccttcattcggctctagtgaagtctcgccgtgtagatcgtaaagaccgtcgtcgtgtggctgaggccaacaaatatttcacacttgagtcggcaattgctcttttcctcagcttcttcatcaacctttttgtggtcgccgttttcgcacatggactttaccagaaaacaaatgcggatgttagagaaatgtgcatagccagacacgacatcccggatgcagatatattcccaaataacactgaaccagttgagggtggaatttatctcggatgccagtttggagctattgcaatgtttatctggggaattggaatattcgcagctggacagtcatcaacaatgacaggaacatatacaggacagtttgtgatggaaggatttgtgaaaattgagtggcccaaatggaagagagttctaattacaagagcgattgcaatcactccaaccctcgtgctcacgttttattcgcaaggagttcaaaacttaacaggaatgaatgatttcttaaattgtgttcaaatgattcaacttccgtttgcactaattccaatcatcacatttacctcgagccgaaaaataatgcacgacttcagaagctctaaagtttttcaaatttttgctctgatcacttctgcgttaattttatcgattaacgtttattttatttctgactacgtgttttctcgacttggtagtgagtggtatatcataatggttcttgctccgatcacctttgcatacgtgctctttgtgttatatctagcactatactgtttggtctcttgtgaaattatcccagacaccgtttccatccgaggattcagctttaacaagtcatacgaaaacgatgctccgtggttggcggttgattcatcagcagtccatgacaatgcaggatatcaataa

SEQ ID NO: 66 - protein, 557 aa, Caenorhabditis elegans
MASSNNDGPIEPEAEPWRITQNDHLEQDLLEEDAESQERVDIPVDDVEKAFSFKKLWAFTGPGFLMSIAYLDPGNIESDLQSGAQAAYKLLWVLLSAHIIGMLLQRMSARLGVVSGKHMAEVAYQFYPRLPRIILWLMIEIAIVCSDMQEVIGTAIAIFLLSKGFVPLYVGVFITILDTFTFLLIDRYGIRKLELIFGFLILTMTVSFGYEFVVVKPPIGEVISGMVVPWCAGCGKGEFMQAISVVGAVIMPHNLYLHSALVKSRRVDRKDRRRVAEANKYFTLESAIALFLSFFINLFVVAVFAHGLYQKTNADVREMCIARHDIPDADIFPNNTEPVEGGIYLGCQFGAIAMFIWGIGIFAAGQSSTMTGTYTGQFVMEGFVKIEWPKWKRVLITRAIAITPTLVLTFYSQGVQNLTGMNDFLNCVQMIQLPFALIPIITFTSSRKIMHDFRSSKVFQIFALITSALILSINVYFISDYVFSRLGSEWYIIMVLAPITFAYVLFVLYLALYCLVSCEIIPDTVSIRGFSFNKSYENDAPWLAVDSSAVHDNAGYQ

SEQ ID NO: 67 - DNA, 1707 bp, Homo sapiens
  CDS (1)..(1707)
atggtgctgggtcctgaacagaagatgtcagatgacagtgtttctggagatcatggggagtctgccagtcttggtaacatcaaccctgcctatagtaatccctctctttcacagtcccctggggactcagaggagtacttcgccacttactttaatgagaagatctccattcctgaggaggagtactcttgttttagctttcgtaaactctgggctttcaccggaccaggttttcttatgagcattgcctacctggatccaggaaatattgaatccgatttgcagtctggagcagtggctggatttaagttgctctggatccttctgttggccacccttgtggggctgctgctccagcggcttgcagctagactgggagtggttactgggctgcatcttgctgaagtatgtcaccgtcagtatcccaaggtcccacgagtcatcctgtggctgatggtggagttggctatcatcggctcagacatgcaagaagtcattggctcagccattgctatcaatcttctgtctgtaggaagaattcctctgtggggtggcgttctcatcaccattgcagatacttttgtatttctcttcttggacaaatatggcttgcggaagctagaagcattttttggctttctcatcactattatggccctcacatttggatatgagtatgttacagtgaaacccagccagagccaggtactcaagggcatgttcgtaccatcctgttcaggctgtcgcactccacagattgaacaggctgtgggcatcgtgggagctgtcatcatgccacacaacatgtacctgcattctgccttagtcaagtctagacaggtaaaccggaacaataagcaggaagttcgagaagccaataagtactttttcattgaatcctgcattgcactctttgtttccttcatcatcaatgtctttgttgtctcagtctttgctgaagcattttttgggaaaaccaacgagcaggtggttgaagtctgtacaaataccagcagtcctcatgctggcctctttcctaaagataactcgacactggctgtggacatctacaaagggggtgttgtgctgggatgttactttgggcctgctgcactctacatttgggcagtggggatcctggctgcaggacagagctccaccatgacaggaacctattctggccagtttgtcatggagggattcctgaacctaaagtggtcacgctttgcccgagtggttctgactcgctctattgccatcatccccactctgcttgttgctgtcttccaagatgtagagcatctaacagggatgaatgactttctgaatgttctacagagcttacagcttccctttgctctcatacccatcctcacatttacgagcttgcggccagtaatgagtgactttgccaatggactaggctggcggattgcaggaggaatcttggtccttatcatctgttccatcaatatgtactttgtagtggtttatgtccgggacctagggcatgtggcattatatgtggtggctgctgtggtcagcgtggcttatctgggctttgtgttctacttgggttggcaatgtttgattgcactgggcatgtccttcctggactgtgggcatacgtgccatctgggattgacagctcagcctgaactctatcttctgaacaccatggacgctgactcacttgtgtctagatga

SEQ ID NO: 68 - protein, 568 aa, Homo sapiens
MVLGPEQKMSDDSVSGDHGESASLGNINPAYSNPSLSQSPGDSEEYFATYFNEKISIPEEEYSCFSFRKLWAFTGPGFLMSIAYLDPGNIESDLQSGAVAGFKLLWILLLATLVGLLLQRLAARLGVVTGLHLAEVCHRQYPKVPRVILWLMVELAIIGSDMQEVIGSAIAINLLSVGRIPLWGGVLITIADTFVFLFLDKYGLRKLEAFFGFLITIMALTFGYEYVTVKPSQSQVLKGMFVPSCSGCRTPQIEQAVGIVGAVIMPHNMYLHSALVKSRQVNRNNKQEVREANKYFFIESCIALFVSFIINVFVVSVFAEAFFGKTNEQVVEVCTNTSSPHAGLFPKDNSTLAVDIYKGGVVLGCYFGPAALYIWAVGILAAGQSSTMTGTYSGQFVMEGFLNLKWSRFARVVLTRSIAIIPTLLVAVFQDVEHLTGMNDFLNVLQSLQLPFALIPILTFTSLRPVMSDFANGLGWRIAGGILVLIICSINMYFVVVYVRDLGHVALYVVAAVVSVAYLGFVFYLGWQCLIALGMSFLDCGHTCHLGLTAQPELYLLNTMDADSLVSR

SEQ ID NO: 69 - DNA, 1521 bp, Drosophila sp.
  CDS (1)..(1521)
atggtggtgcgtccgtacaacgatgagctccggtacctggagaaagtgagcgaccactgctggcgcatcaagaagggcttccagccaaatatgaatgtggaggggtgtttctatgtgaacagccggctggagcgcctgatgctggaggagctgaagaactcctgtcgcccgggcgcagtgggtggcttcctgcctggcgtcaagcagatagccaatgtggccgcgttgccgggcatcgtgggcaggtccattggactgcccgacattcattccggctacggatttgccatcgggaacatggctgctttcgacatgaacgatccgctgtccgttgtaagtcccggcggcgtgggtttcgacatcaactgtggcgtgcgtctgctgcgcacgaatctgtacgagaaggatgtgcagccggtgaaggagcaactggcgcagtccctgttcgatcacatacccgtgggtgtgggctccaagggcatcatacccatgaatgcccgcgatctggaggaggccctcgaaatgggcatggactggtcgctgcgcgagggatacgtgtgggcggaggacaaggagcattgcgaggagtacggccgcatgctgaacgccgatcccgccaaggtgagcatgcgggccaagaagcgagggctgccccagctgggcactctgggtgcgggcaatcactacgccgagatccaggtggtggacgaaatctacgacaagtggagcgcctccaagatgggcatcgaggagaagggccaggtggtggtgatgattcactcgggcagtcgtggcttcggccaccaggtcgctaccgacgccctggtccagatggagaaggccatgaagcgggacaagatcgagaccaatgaccggcagctggcctgcgccaggatcaattcggtggagggacaggactacttgaaggccatggcggcggctgcgaactttgcctgggtgaatcgcagctccatgacattcctcacccgtcaagcgtttgccaagatgtttaacaccacacccgatgatctcgacatgcacgttatctatgacgtttcgcacaatattgccaaggtggagaaccacatggtggacggcaaggagcggaagctgttggttcaccggaagggctccacgcgcgccttcccgccacaccatcccctgatcccagtggactatcagcttaccgggcagccagtcctcgtcggtggaaccatgggcacttgcagttacgtgctaactggaacggagcagggcatgcaggagacgttcggtagcacttgccacggagcgggtcgtgcactatctcgagccaaatcccggcgcaatctggactacaaggatgtgctggacaagctggaccagttgggcatcgccatacgcgtggcctcgcccaaactggtcatggaggaggcacccgaatcttacaaggacgtgaccgatgtggtcgacacctgtcacgcagctggcatcagcaaaaagtgcatcaagatgcgcccaattgcagttatcaagggctaa

SEQ ID NO: 70 - protein, 506 aa, Drosophila sp.
MVVRPYNDELRYLEKVSDHCWRIKKGFQPNMNVEGCFYVNSRLERLMLEELKNSCRPGAVGGFLPGVKQIANVAALPGIVGRSIGLPDIHSGYGFAIGNMAAFDMNDPLSVVSPGGVGFDINCGVRLLRTNLYEKDVQPVKEQLAQSLFDHIPVGVGSKGIIPMNARDLEEALEMGMDWSLREGYVWAEDKEHCEEYGRMLNADPAKVSMRAKKRGLPQLGTLGAGNHYAEIQVVDEIYDKWSASKMGIEEKGQVVVMIHSGSRGFGHQVATDALVQMEKAMKRDKIETNDRQLACARINSVEGQDYLKAMAAAANFAWVNRSSMTFLTRQAFAKMFNTTPDDLDMHVIYDVSHNIAKVENHMVDGKERKLLVHRKGSTRAFPPHHPLIPVDYQLTGQPVLVGGTMGTCSYVLTGTEQGMQETFGSTCHGAGRALSRAKSRRNLDYKDVLDKLDQLGIAIRVASPKLVMEEAPESYKDVTDVVDTCHAAGISKKCIKMRPIAVIKG

SEQ ID NO: 71 - DNA, 1518 bp, Danio sp.
  CDS (1)..(1518)
atgagtcgctcttacaacgatgagctccagtatctggataaaatacacaaaaactgctggcggatcaagaagggtttcgtgccgaatatgctggtggaaggagtgttttatgtcaatgacccgctggaaaagctgatgttcgaggagctgagaaacgcctgtcgcggaggagggtttggaggtttcttacctgcgatgaagcagattgggaatgtggccgctctgccaggaatcgtgcaccggtcgatcggtttaccggacgttcactcaggatacggattcgctatcgggaacatggcagcgttcgacatggagaatccggacgcagtcgtctctccaggcggtgtgggtttcgatattaactgtggtgttcgtctgctgcgcacaaacctggatgagggcgacgttcagccggtgaaggagcagctggcacagtctctcttcgaccacatccctgtcggagtcggctccaagggcgtcattcctatgggtgcaaaggacctggaggaggcgttggagatgggtgtggactggtctctgagggagggatatgcctgggcggaggataaagagcactgtgaggagtacggacgcatgctgcaggccgacccaaacaaagtctcctccaaagccaagaagagaggactgccacagttgggaactctgggtgcaggaaaccactacgcagagattcaggtggtggacgagatctacaatgattacgccgccaagaagatgggcatcgatcataaagggcaggtgtgtgtgatgatccacagcggcagccgaggactcggacatcaggtggccaccgacgctctggtggcgatggagaaggccatgaagcgcgaccgcatcacagtaaacgaccggcagctagcgtgcgcgcgcatcacgtcagaagagggacaggattatctgaagggaatggcggcagcaggaaactacgcctgggtcaaccgatcctccatgaccttcctcacacgacaggcgttctccaaagtgttcagcaccacaccagatgatctggacatgcacgtgatctacgacgtctcgcacaacatcgccaaagtggaggagcacatggtggacggccggcagaaaacactgctggtgcataggaagggctccaccagagcgtttcctccacaccatccactcatacctgtagactatcagctgaccggtcagccagtcctgattggaggaaccatgggcacctgcagttacgtgctcacaggcacagagcagggcatgacagagacgttcggcaccacatgtcacggcgctggccgagctttatccagagccaaatccagacgcaacctggacttccaggatgttctggataaactggcagacatgggcatcgctattagagtggcgtcaccgaagctggtgatggaggaggctcccgagtcctacaagaacgtgacagacgtggtgaacacatgccatgatgccggcatcagcaaaaaagccatcaaactcagacccatcgctgtgattaaaggttaa

SEQ ID NO: 72 - protein, 505 aa, Danio sp.
MSRSYNDELQYLDKIHKNCWRIKKGFVPNMLVEGVFYVNDPLEKLMFEELRNACRGGGFGGFLPAMKQIGNVAALPGIVHRSIGLPDVHSGYGFAIGNMAAFDMENPDAVVSPGGVGFDINCGVRLLRTNLDEGDVQPVKEQLAQSLFDHIPVGVGSKGVIPMGAKDLEEALEMGVDWSLREGYAWAEDKEHCEEYGRMLQADPNKVSSKAKKRGLPQLGTLGAGNHYAEIQVVDEIYNDYAAKKMGIDHKGQVCVMIHSGSRGLGHQVATDALVAMEKAMKRDRITVNDRQLACARITSEEGQDYLKGMAAAGNYAWVNRSSMTFLTRQAFSKVFSTTPDDLDMHVIYDVSHNIAKVEEHMVDGRQKTLLVHRKGSTRAFPPHHPLIPVDYQLTGQPVLIGGTMGTCSYVLTGTEQGMTETFGTTCHGAGRALSRAKSRRNLDFQDVLDKLADMGIAIRVASPKLVMEEAPESYKNVTDVVNTCHDAGISKKAIKLRPIAVIKG

SEQ ID NO: 73 - DNA, 1518 bp, Bos sp.
  CDS (1)..(1518)
atgagtcgcagttataatgatgagctgcagttcttggaaaagatcagtaagaactgctggagaatcaagaagggcttcgtgcccaacatgcaggttgaaggagttttctatgtgaatgattctctggaaaaattaatgtttgaagaattaaggaatgcctgtcgaggtggtggtgttggtggcttcctgccagccatgaaacaaattggcaatgtggccgccctgcctgggattgttcatcgatccatcggtcttcctgatgtccattcaggttatgggtttgctattggaaatatggcagcctttgatatgaacgaccctgaagcagtggtatccccaggtggtgttgggtttgacattaactgtggtgtccgcttgctgagaaccaatttagatgaaagtgatgttcagcctgtgaaagagcaacttgcccaagctatgtttgaccacattcctgtgggagtggggtcaaaaggtgtcatcccaatgaatgccaaagacttggaggaggccttggagatgggtgtggactggtccctgagagaaggctatgcctgggcagaggacaaggagcactgtgaggagtatggaaggatgctgcaagctgatcccaataaagtctcagccagggctaaaaaaagaggccttccccagttggggactctgggagcaggcaaccactatgcagaaatccaggttgtggatgagattttcaacgagtatgctgctaagaaaatgggcattgaccataagggacaggtgtgtgtgatgatccacagtggaagcagaggcttgggccaccaagttgccacagatgcacttgtagctatggaaaaagccatgaagagagacaagattatagtcaatgaccgtcagttggcttgtgctcgaattgcttccccagagggtcaggactacctgaagggaatggcagcggctgggaactatgcctgggtcaaccgctcttccatgaccttcttaacccgtcaggcttttgccaaggtcttcaacacaacccctgatgacttggacctgcatgtgatctatgatgtttctcacaatattgccaaagtagaacagcatgtggtggacgggaaggagcggactctgttagtacacaggaaggggtccacccgagccttccctcctcaccatcccctcattgcggttgattaccaacttaccggacaaccagtgctcattggtggcaccatgggaacctgtagctatgttcttactggtactgagcagggcatgactgaaacctttggaacaacttgtcatggagcgggccgtgcactgtcccgagcaaagtcaagacgtaatttagatttccaggatgtcctcgacaaattggcagacatgggaattgcaatccgtgtcgcctcacccaagctggtaatggaagaggcccctgagtcctataagaacgtgacggatgtggtgaacacctgccatgatgccggaatcagcaagaaggccattaaactgaggccaattgctgttatcaaaggatag

SEQ ID NO: 74 - protein, 505 aa, Bos sp.
MSRSYNDELQFLEKISKNCWRIKKGFVPNMQVEGVFYVNDSLEKLMFEELRNACRGGGVGGFLPAMKQIGNVAALPGIVHRSIGLPDVHSGYGFAIGNMAAFDMNDPEAVVSPGGVGFDINCGVRLLRTNLDESDVQPVKEQLAQAMFDHIPVGVGSKGVIPMNAKDLEEALEMGVDWSLREGYAWAEDKEHCEEYGRMLQADPNKVSARAKKRGLPQLGTLGAGNHYAEIQVVDEIFNEYAAKKMGIDHKGQVCVMIHSGSRGLGHQVATDALVAMEKAMKRDKIIVNDRQLACARIASPEGQDYLKGMAAAGNYAWVNRSSMTFLTRQAFAKVFNTTPDDLDLHVIYDVSHNIAKVEQHVVDGKERTLLVHRKGSTRAFPPHHPLIAVDYQLTGQPVLIGGTMGTCSYVLTGTEQGMTETFGTTCHGAGRALSRAKSRRNLDFQDVLDKLADMGIAIRVASPKLVMEEAPESYKNVTDVVNTCHDAGISKKAIKLRPIAVIKG

SEQ ID NO: 75 - DNA, 1518 bp, Mus sp.
  CDS (1)..(1518)
atgagtcgtaactacaacgatgagctacagttcttggacaagatcaataaaaactgctggaggatcaagaagggctttgtgcccaacatgcaggttgaaggagtgttttatgtgaatgatgctctggaaaaactaatgtttgaggaattaaggaacgcctgtcgaggtggtggtgttggtggctttctgccagccatgaagcagattggcaatgtggcagccctgcctggaatagttcatcggtctatcgggcttcctgatgtccattcaggctatgggtttgccatagggaacatggctgcctttgatatgaatgaccctgaggccgttgtatccccaggtggtgtcggatttgatattaactgtggtgtccgcttgctaagaaccaatttagatgagagcgatgtacagcctgtgaaggaacaacttgcccaagctatgtttgaccacatccctgttggggtgggatcaaaaggtgtcattccaatgaatgccaaagacttggaggaggcattggagatgggggtggactggtccctgagggaaggctatgcctgggctgaagacaaggagcactgtgaggagtatggaaggatgctgcaagccgaccccaataaggtctcacccagggcaaagaaaaggggccttcctcagttggggaccctgggagcaggcaaccattatgcagaaatccaggttgtagatgagattttcaatgagtatgccgccaagaagatgggcatcgaccataagggacaggtgtgtgtgatgatccacagtggaagcagaggcttgggccaccaagtagctacagatgcactggtagctatggaaaaggccatgaagagagacaagattatagtcaatgaccggcagttggcttgtgctcggattgcatccccagagggacaagactatctaaagggaatggctgcagctggaaactacgcctgggttaaccgctcctctatgaccttcttaacccgtcaggcttttgccaaagtcttcaacacaacccctgatgacctggacctgcatgtgatctatgatgtgtcgcacaatatcgccaaagtggagcagcacgtggtggatgggaaggaacggacgctgctggtgcacaggaagggatccacccgtgctttcccgcctcaccaccccctcattgctgtggattatcaactcacaggacaaccagtgcttattggtggcaccatggggacctgtagttacgttctgactggcactgaacaaggcatgactgagacctttggaacaacctgtcatggagcgggccgtgctttgtccagagcaaaatcacgtcgtaacttagatttccaagatgtcttagacaaactggcagacatgggaattgcaatccgggttgcttcccccaagctggttatggaagaggcaccagagtcctataagaatgtgacagacgtcgtgaacacctgccatgatgctgggatcagcaagaaggccattaaactgagaccaattgctgttattaaagggtag

SEQ ID NO: 76 - protein, 505 aa, Mus sp.
MSRNYNDELQFLDKINKNCWRIKKGFVPNMQVEGVFYVNDALEKLMFEELRNACRGGGVGGFLPAMKQIGNVAALPGIVHRSIGLPDVHSGYGFAIGNMAAFDMNDPEAVVSPGGVGFDINCGVRLLRTNLDESDVQPVKEQLAQAMFDHIPVGVGSKGVIPMNAKDLEEALEMGVDWSLREGYAWAEDKEHCEEYGRMLQADPNKVSPRAKKRGLPQLGTLGAGNHYAEIQVVDEIFNEYAAKKMGIDHKGQVCVMIHSGSRGLGHQVATDALVAMEKAMKRDKIIVNDRQLACARIASPEGQDYLKGMAAAGNYAWVNRSSMTFLTRQAFAKVFNTTPDDLDLHVIYDVSHNIAKVEQHVVDGKERTLLVHRKGSTRAFPPHHPLIAVDYQLTGQPVLIGGTMGTCSYVLTGTEQGMTETFGTTCHGAGRALSRAKSRRNLDFQDVLDKLADMGIAIRVASPKLVMEEAPESYKNVTDVVNTCHDAGISKKAIKLRPIAVIKG

SEQ ID NO: 77 - DNA, 1518 bp, Rattus sp.
  CDS (1)..(1518)
atgagtcgtaactacaacgatgagctacagttcttggacaagatcaataagaactgctggaggatcaagaagggctttgtgcccaacatgcaggttgaaggggtgttttatgtgaatgacgctctggaaaagctcatgtttgaggagttacggaatgcctgtcgaggtggtggtgttggtggcttcctgccagccatgaagcagattggcaatgtggcagccctgcctggaatagttcatcggtctattgggcttcctgatgtccactcaggctacgggtttgccatagggaacatggctgcctttgatatgaatgaccctgaggcagttgtatccccaggtggtgtcggatttgatattaactgtggtgtccgcttgctaaggaccaatttagatgagagcgatgtacagcctgtgaaggaacaacttgcccaagctatgtttgaccacatccctgtcggggtgggatcgaaaggtgtcattccaatgaatgccaaagacttggaggaggcattggagatgggtgtggactggtccctaagagaaggctatgcctgggctgaggacaaggagcactgtgaggagtatggaaggatgctccaagccgaccccaataaggtctcacccagagcaaagaaaaggggccttcctcagttggggaccctgggagcaggcaaccattatgcagagatccaggttgtagatgagattttcaacgagtatgctgccaagaagatgggcatcgaccataagggacaggtgtgcgtgatgatccacagcgggagcagaggcttgggccatcaagtagctacagacgcactggtagctatggagaaagccatgaagagagacaagattatagtcaatgaccggcagctggcgtgtgctcggattgcatccccagagggacaagactatctaaagggaatggctgccgctggaaactgtgcctgggttaaccgctcgtctatgaccttcttaacccgtcaggcttttgccaaagtcttcaacacaacccctgacgacctggacctgcatgtgatttatgatgtttctcacaacatcgccaaagtggagcagcacgtggtagacggaaaggagcggacgctgttggtgcacaggaaagggtccacccgcgctttccctcctcaccatcccctcattgctgttgattaccagctcactggacaaccagtgcttatcggtggcaccatggggacctgtagttatgttctgactggcactgaacaaggcatgactgagacctttggaacaacctgtcatggagcgggccgtgctttgtccagagcaaaatcacgtcgtaatttagatttccaagatgtcttagacaagctggcagacatgggaatcgccatccgggttgcgtcccccaagctggttatggaagaggctccagaatcatataagaatgtgacagacgtcgtgaacacttgccatgatgctgggatcagcaagaaggccattaaactgagaccaattgctgttattaaaggatag

SEQ ID NO: 78 - protein, 505 aa, Rattus sp.
MSRNYNDELQFLDKINKNCWRIKKGFVPNMQVEGVFYVNDALEKLMFEELRNACRGGGVGGFLPAMKQIGNVAALPGIVHRSIGLPDVHSGYGFAIGNMAAFDMNDPEAVVSPGGVGFDINCGVRLLRTNLDESDVQPVKEQLAQAMFDHIPVGVGSKGVIPMNAKDLEEALEMGVDWSLREGYAWAEDKEHCEEYGRMLQADPNKVSPRAKKRGLPQLGTLGAGNHYAEIQVVDEIFNEYAAKKMGIDHKGQVCVMIHSGSRGLGHQVATDALVAMEKAMKRDKIIVNDRQLACARIASPEGQDYLKGMAAAGNCAWVNRSSMTFLTRQAFAKVFNTTPDDLDLHVIYDVSHNIAKVEQHVVDGKERTLLVHRKGSTRAFPPHHPLIAVDYQLTGQPVLIGGTMGTCSYVLTGTEQGMTETFGTTCHGAGRALSRAKSRRNLDFQDVLDKLADMGIAIRVASPKLVMEEAPESYKNVTDVVNTCHDAGISKKAIKLRPIAVIKG